SEQUENCE LISTING (1 sequence)

SEQ ID NO: 1 - protein, 167 aa, rat
MRGSVSQEEVKKWAESLENLINHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLTNPSSCGAEKQKGAKSSADCTSLVPQCAHHHHHHHP

(19) United States
(12) Patent Application Publication
(10) Pub. No.: US 2003/0186413 A1
(43) Pub. Date: Oct. 2, 2003

(54) Structure of a free regulator of G-protein signaling (RGS4) and methods of identifying agonists and antagonists using same

(75) Inventors: Robert Powers, Westford, MA (US); Franklin J. Moy, Arlington, MA (US)

Correspondence Address: WYETH, PATENT LAW GROUP, FIVE GIRALDA FARMS, MADISON, NJ, 07940, US

(73) Assignee: American Home Products Corporation, Madison, NJ

(21) Appl. No.: 09/942,055

(22) Filed: Aug. 29, 2001

Related U.S. Application Data
(63) Continuation of application No. 09/569,836, filed on May 11, 2000, now abandoned.

(51) Int. Cl. C12N 9/10; G06F 19/00; G01N 33/48; G01N 33/50

(52) U.S. Cl. 435/193; 702/19

(57) ABSTRACT

The present invention provides a solution structure of free RGS4 determined using NMR techniques. The structure includes a Gα binding site and an allosteric binding site. The structural information provided can be employed to identify, select or design agonists and antagonists of RGS4 activity. The invention includes two dimensional and three dimensional models and representations of the structure of free RGS4 based upon structural coordinates that are provided that are useful in the methods described.

### BACKGROUND OF THE INVENTION

A variety of biochemical processes, particularly those involving protein-protein interactions, are believed to be mediated by an induced conformational change in the protein target. The resulting structural change in the protein is then used to explain a modification in its function (e.g., enzymatic activity) or its affinity for another protein in the biological system. Conformational change has been proposed to occur in the cascade of steps associated with certain signal transduction pathways in eukaryotic cells. A ubiquitous component of such signal transduction pathways is a heterotrimeric guanine nucleotide-binding protein (G-protein) coupled to a cell surface receptor (for reviews see references 1-4 and 72). G-proteins relay signals initiated by various stimuli including photons, odorants, and a number of hormones and neurotransmitters. A variety of diseases are caused by defects in G-protein activity. G-proteins exist as heterotrimeric complexes of α, β, and γ subunits. The α-subunit (Gα) is weakly bound to a dimer (Gβγ) in which the β-subunit is tightly bound to the γ-subunit. Gα is also associated with the intracellular carboxy terminal tail of a seven-helical transmembrane receptor. G-proteins transfer signals from more than 1000 receptors with various Gα subtypes regulating a variety of distinct downstream signaling pathways. Guanine nucleotide binding and GTPase function within the Gα domain to regulate the activity of G-proteins.

The G-protein signaling process is typically initiated by the binding of an agonist to the cell surface receptor resulting in an induced conformational change in the G-protein. The G-protein structural chance affects the guanine nucleotide affinity of Gα, so that it preferentially binds GTP and Mg²⁺ over GDP. Numerous x-ray structures for G_{iα1} during the various stages of the GTPase cycle have been used to identify regions of induced conformational change (5-8). In particular, the Gα guanine nucleotide binding site is composed of three distinct “switch” regions: residues V179-V185 in switch I, residues Q204-H213 in switch II and residues A235-N237 in switch III, which undergo conformational changes upon GTP hydrolysis. The Gα surface that binds the Gβγ dimer contains switch I and switch II regions. In the active Gα-GTP-Mg²⁺ complex, a conformational change in switch I is associated with binding Mg²⁺, and switch II and switch III regions become well ordered due to ionic interactions between the two switch regions. As a result of the formation of the Gα-GTP-Mg² complex, modifications in the structure of the three “switch” regions facilitate dissociation of Gα from Gβγ. The released subunits are then available to interact with a variety of target proteins to elicit the desired response. Termination of the signal results when the process is reversed by the hydrolysis of GTP bound to Gα. Reassociation of Gα with Gβγ results in the inactivation of the G-protein. Therefore the duration of the G-protein signal is directly dependent on the GTPase activity of the Gα protein.

Regulators of G-protein signaling (RGS) affect the intensity and duration of the G-protein signal cascade by binding to the active Gα-GTP-Mg² complex and inducing a 50-fold increase in the rate of GTP hydrolysis (For reviews see references 9-13). Conversely, RGS proteins have little or no affinity for the inactive Gα-GDP complex. Thus, RGS proteins act as attenuators of the induced G-protein signal by increasing the rate of inactivation of the G-protein and termination of the signal. RGS proteins may exhibit additional biological function, e.g., RGS4 is reported to block activation of GTP-Gα by effectors (83). The RGS family, including RGS4, GAIP (human Gα-interacting protein), RGS1, RGS10, and RGS16, among others, contains more than 20 known members where specificity for Gα subtypes has been demonstrated and appears to be associated with subtle sequence differences (8, 14). The RGS family contains significant structural diversity, however, all RGS proteins are characterized by a conserved domain of about 130 amino acids which may be separated by linker regions of varying lengths. Recently, the RGS family has been reported to comprise at least six separate subfamilies designated A-F with unique structural features (Zheng, B. et al. (1999) (86). RGS4 exhibits structural features of RGS subfamily A. Subfamily-specific structural features may be associated with subfamily-specific functions, e.g., differences in Gα binding specificity among RGS proteins, membrane association of RGS protein, or functions exhibited by RGS proteins in addition to GAP activity. RGS4 is believed to function to attenuate induced G-protein by stabilizing the transition.

RGS proteins are widely expressed in eukaryotic cells, including human cells (13). At least one RGS protein is found in tissue of each human organ and many tissues express multiple RGS proteins. Additionally, members of the RGS family are specifically expressed in the human brain, where RGS4 is perhaps the most widely distributed and highly expressed RGS subtype (15, 16). RGS expression has been correlated with a response to induced seizures, which indicates that regulation of RGS expression is an adaptive response in the brain signal transduction pathway to compensate for desensitization and sensitization of G-protein-coupled receptor function (16). In addition to regulation of the response to neurotransmiters, RGS activity has been associated with a variety of cellular functions including: cell proliferation, cell differentiation, membrane trafficking and embryonic development (9, 10, 12, 17).

An x-ray structure of RGS4 bound to Giα [8], site-directed mutagenesis [18-20] and biochemical studies [17, 21] suggest that RGS4 binds preferentially to the Gα-GTP-Mg²⁺ complex and stabilizes the transition state structure of the switch regions facilitating hydrolysis of GTP. Since the functional result of RGS4 binding to Giα1 is induction of GTP hydrolysis by Giα1, it is reasonable to anticipate that the conformational change upon complex formation with RGS4 primarily occurs in G_{iα1}. However, the x-ray crystal structure of Giα1 in the RGS4-Giα1 complex exhibits only a 0.6 Å rms difference from that of Giα1 in Giα1-AIF₄⁻ which is trapped in the proposed transition state for GTP hydrolysis. This comparison indicates that there is no significant conformational change in Giα1. On the other hand, analysis of the RGS4-Giα1 complex x-ray structure indicates that RGS4 binding to Giα1 induces a decrease in the mobility of the switch regions of Giα1. In these regions, critical interactions occur between N82 of RGS4 (employing the numbering of FIG. 1) with the switch regions I and II of Giα1 and between T182 of Giα1 with a Gα binding pocket on RGS4. The RGS4 residue N82 has been identified as critical for facilitating the intrinsic Giα1 GTPase activity presumably by stabilizing the switch regions and substrate binding (19, 20). Similar changes in the switch regions are observed between the Gα-GTP-Mg² complex and the Gα-GDP complex (2), suggesting that a conformational change in RGS4 may contribute to regulation of G-protein signaling.

de Alba, E. et al. (1999) (87) reports the solution structure of human GAIP as determined by NMR techniques. The structure calculation used dipolar couplings of the oriented protein in two different liquid crystal media. The GAIP solution structure was compared to that of the rat RGS4-Gᵢα₁ x-ray structure (8). The reference suggests that GAIP-L187 participates in Gα-RGS binding and may also be important in the folding and stability of the RGS protein. It is also suggested that GAIP-S156 plays a role in GAIP stability. GAIP-S156 has been identified as a subfamily-specific residue for the RGS subfamily. A [GAIP, Ret-RGS1, RGS21] (86) and Wang et al. (89). In RGS subfamily B which includes RGS4, the core amino acid corresponding to GAIP S156 is RGS N82 (as numbered in FIG. 1 and N128 as numbered in Tesmer et al. (8)). The core region of GAIP is reported to have only 60% sequence identity to the core of RGS4. Any differences observed between these two structures are at least in part due to the differences in amino acid sequence.

It is thus desirable to provide structural information for free RGS4 to better understand the mechanism of the regulation of G-protein signaling. More specifically, such structural information allows a direct comparison between the solution structure of RGS4 and the x-ray structure of the RGS4-Gα complex to determine which conformational changes occur in RGS proteins on binding to Gα. The structural information and comparison can be employed to identify factors (chemical or biochemical species) that affect G-protein signaling by interaction with RGS proteins or their complexes with Gα. The structurally information can be of particular use in the identification and rational design of agonists and antagonists of free RGS and RGS/Gα complex activity.

### SUMMARY OF THE INVENTION

The present invention provides the three-dimensional solution structure of a free (i.e., not complexed) RGS protein of subfamily B, specifically that of free RGS4, as determined by NMR (nuclear magnetic resonance) spectroscopy. Particularly, the invention provides the three-dimensional solution structure of a Gα binding site of an RGS subfamily B protein. The Gα binding site of RGS subfamily B is exemplified by the three-dimensional structure of the RGS4 Giα1 binding site comprising the RGS4 protein residues D117, S118 and R121. The invention also provides the three-dimensional structure of the α6-α7 region of a free RGS subclass B which region exhibits a significant conformational change on binding of RGS to Gα. Binding at the α6-α7 region of RGS protein can effect the function of RGS protein in G-protein signaling. Further, the invention identifies and provided the three-dimensional structure of an allosteric binding site in an RGS protein. Binding at this allosteric site can affect the regulation of G-protein signaling. An allosteric binding site in the RGS protein is exemplified by the allosteric binding site in RGS4 located in the α1 and α2 helical regions of free RGS4 and in the tight turn located between the two helical regions. More specifically, the allosteric binding site in RGS4 comprises the residues V10, W13, L17, 120, H23, E24, C25 and T132.

The three dimensional structure of free RGS4 in solution, including the Gα binding site, the C-terminus α₆-α₇ region of free RGS4, and the allosteric binding site in free RGS4 are provided by the relative atomic structural coordinates given in Table 2 as obtained by NMR spectroscopy. Also provided are the ¹⁵N, ¹³C, ¹³CO and ¹H NMR assignments for free RGS4 (Table 1) which are employed in the determination of its secondary and three-dimensional structure. These assignments are also useful in methods for identifying or detecting chemical and biochemical species that bind to RGS and which can affect RGS function or RGS-Gα function and are particularly useful for identifying or detecting species that bind to RGS subclass B which can affect its function or RGS subfamily B-Gα function.

The invention further provides a representation or model of all or part of the three-dimensional structure of a free RGS subfamily B protein comprising a data set of relative atomic structural coordinates embodying the three-dimensional structure of free RGS4 protein. The invention also provides a data set of relative atomic structural coordinates embodying the three-dimensional structure of the Gα binding site in an RGS subfamily B protein. The invention further provides a data set of relative atomic structural coordinates embodying the α₆-α₇ region of an RGS subfamily protein. In addition, the invention provides a data set of relative atomic structural coordinates embodying an allosteric binding site in an RGS subfamily B protein. The data set and any structural representation or model of a free RGS subfamily B, its Gα binding site its α₆-α₇ region or the allosteric binding site in RGS subfamily B created or generated using the data set provided herein can be employed to identify, select or rationally design factors, e.g., chemical or biochemical species, which affect RGS function or activity or RGS/Gα complex activity or function. Further, the data set, structural representation or model of the Gα binding site can also be used to identify, select or rationally design species which affect Gα function by binding to Gα. The data set and structural representations and models provided by this invention are particularly useful for the identification of agonists or antagonists of RGS function or RGS/Gα complex function or activity.

The data set, including subsets of data embodying the Gα binding site, the α6-α7 region and the allosteric binding site, provided herein was determined by NMR analysis. However, any known method can be employed to provide the structural data. In one embodiment, the data set embodies the structure of free RGS4 in solution. In certain embodiments, the data set comprises one or more portions, of the structure of free RGS4. Of particular interest are those portions of the structure of RSG4 which function in RSG-regulation of G-protein signaling or more specifically which affect binding of RGS to Gα or which affect the biological function or activity of the RGS-Gα complex. Also of interest are those portions of the structure of RSG4 to which candidate agonists and antagonists of RSG bind to affect its biological function.

Any available method may be used to construct a structural representation or model from the NMR—derived data disclosed herein or from data obtained from an independent structural analysis of free RGS4. Such a model or representation can be generated or constructed from the available analytical data points using software packages such as HKL, CHARMM, MOSFILM, XDS, CCP4, SHARP, PHASES, HEAVY, XPLOR, TNT, NMRCOMPASS, NMRPIPE, DIANA, NMRDRAW, FELIX, VNMR, MADIGRAS, QUANTA, BUSTER, SOLVE, O, FRODO, XPLOR, RASMOL, and CHAIN, all of which are well-known and available to those in the art. A structural representation or model can be generated from these data using available systems, including, for example, Silicon Graphics, Evans and Sutherland, SUN, Hewlett Packard, Apple Macintosh, DEC, IBM, and Compaq systems. The structural representation or model can be displayed or generated in any two-dimensional or three-dimensional form known in the art for viewing, analyzing, modeling or otherwise representing the structure. The structural representation can be transmitted, conveyed or stored in any known graphic, digital or analog form. Structural representations or models generated with the RGS data provided herein can be combined with structural representations of other chemical and biochemical species (e.g., candidate antagonists or agonists) including x-ray data of RGS-complexes, in order to analyze potential interactions between RGS, particularly RGS subfamily B proteins, and Gα and those species. The data provided herein may also be combined, as illustrated herein, with structural information (including x-ray data) of RGS-complexes, particularly RGS subclass B protein-complexes and particularly those complexes believed to be or believed to model biologically functional complexes.

The present invention relates to the structural data for free RGS4, the Gα binding site of RGS4, the α6-α7 region whose conformation changes on binding of RGS4 to Gα, and allosteric binding sites in RGS4 in any form (for example in digital, tabular, graphic, or pictorial form or as embodied in any representation or model or as embodied in a computer storage medium) and the use of the data (in whatever form) for generating a structural representation or model of free RGS particularly an RGS subfamily B protein, more particularly RGS4, or of the interaction of RGS, an RGS subfamily B protein, and RGS4, with any other chemical or biochemical species, including structural representations or models of RGS interaction with G-protein subunits and of RGS interaction with potential agonists or antagonists of RGS function.

The present invention also provides for a computer system which comprises the structural representation or model of the invention and hardware used for construction, processing and/or visualization of the model of the invention. The solution structural coordinates of RGS4 or portions thereof as provided herein can be stored in or on an appropriate medium for introduction into or use with any computer program or system for generating a representation or model of the structure of an RGS protein, an RGS subclass B protein or RGS4, or for analysis of the interaction of RGS with other chemical or biochemcial species.

The structural coordinates can be stored in a machine-readable form on a machine-readable storage medium, for example, a computer hard drive, diskette, DAT tape, etc., for display as a three-dimensional shape or for other uses involving computer-assisted manipulation of, or computation based on, the structural coordinates or the three-dimensional structures they define. By way of example, the data defining the three dimensional structure of RGS4 of the present invention, or of a portion of RGS4 as disclosed herein, may be stored in a machine-readable storage medium, and may be displayed as a graphical three-dimensional representation of the relevant structural coordinates, typically using a computer capable of reading the data from said storage medium and programmed with instructions for creating the representation from such data.

Accordingly, the present invention provides a machine, such as a computer, programmed in memory with the coordinates of the RGS4 or RGS subfamily B protein, or portions thereof (such as, by way of example, the coordinates of the RSG4 Gα binding site, the α6-α7 region of RGS4, or the allosteric binding site in the α1-α2 region of RGS4), together with a program capable of converting the coordinates into a three dimensional graphical representation of the structural coordinates on a display connected to the machine. A machine having a memory containing such data aids in the rational design or selection of inhibitors or activators of RGS, Gα or RGS-Gα complex activity, including the evaluation of the ability of a particular chemical or biochemical species to favorably associate with RGS, particularly an RGS subclass B protein, as well as in the modeling of compounds, proteins, complexes, etc. related by significant structural or sequence homology to RGS4 or other RGS proteins.

The present invention is additionally directed to a method of determining the three dimensional solution structure of a compound, e.g., a protein or peptide or other chemical or biochemical species (including RGS proteins or portions thereof, or more specifically, RGS subfamily B proteins or portions thereof that are not RGS4) whose structure is unknown, comprising the steps of first obtaining a solution of the protein or peptide whose structure is unknown, and then generating NMR data from this solution. The NMR data from the protein or peptide can then be compared with the known three dimensional structure of RGS4 (or portion thereof, e.g., the Gα binding site) as disclosed herein, and the three dimensional structure of the protein or peptide whose structure is unknown conformed to the known RGS4 structure using standard techniques, such as 2D, 3D and 4D isotope filtering, editing and triple resonance NMR techniques, computer homology modeling as well an adaptation of molecular replacement techniques as applied to NMR data. Alternatively, a three dimensional model of a protein or peptide of unknown structure, but related by sequence similarity to RGS4, may be generated by initial sequence alignment between RGS4 and the protein or peptide, based on any or all amino acid sequence identity, secondary structure elements or tertiary folds, and then generating by computer modeling a three dimensional structure for the molecule using the known three dimensional structure of, and sequence alignment with, RGS4.

Methods are also provided for identifying a species which is an agonist or antagonist of RGS activity, RGS binding to Gα, Gα binding to RGS, or RGS/Gα complex activity, particularly for RGS subfamily B proteins. The method comprises the steps of using a three dimensional structure of free RGS subfamily B protein or a portion (e.g., an RGS4 core protein) thereof as defined by the relative structural coordinates of amino acids encoding the RGS4-core protein to design or select a potential agonist or antagonist, and synthesizing or otherwise obtaining the potential agonist or antagonist. The potential agonist or antagonist may be selected by screening an appropriate database, may be designed de novo by analyzing the steric configurations and charge potentials of the RGS4 Gα binding site, the α₆-α₇ region of RGS4, or an allosteric binding site of RGS4 in conjunction with the appropriate software programs, or may be designed using characteristics of known agonists or antagonists of RGS4, RGS subfamily B, or other RGS proteins in order to create “hybrid” agonists or antagonists. The method of the present invention is preferably used to design or select inhibitors of RGS subfamily B proteins, or RGS subclass B-Gα complex activity, and specifically RGS4 or RGS4-Giα1 complex activity. In a specific embodiment, the potential agonist or antagonist is identified, selected or designed by studying the interaction of candidate species with a three-dimensional model of RGS4 (or a portion thereof or a three-dimensional model of another RGS subfamily B protein (or model thereof) and selecting a species which is predicted by its interaction with the RGS protein or a portion of an RGS protein to act as an agonist or antagonist. Potential antagonists and agonists can be readily tested using various procedures disclosed herein or known in the art to confirm their antagonist or agonist function. Species identified in accordance with such methods are also provided.

Other specific embodiments provide: (1) a process of identifying a substance that inhibits RGS4 activity, RGS4 binding to Giα1, Giα1 binding to RGS4 or RGS4/Giα1 complex activity comprising determining the interaction between a candidate substance and a model of all of part of the structure of free RGS4, or (2) a process of identifying a substance that mimics or promotes RGS4 activity, RGS4 binding to Giα1, Giα1 binding to RGS4 or RGS4/Gα complex activity comprising determining the interaction between a candidate substance and a model of all or part of the structure of free RGS4 by analyzing the steric configuration and charge potential of free RGS4 and comparing these properties to those of a candidate substance. Substances identified in accordance with such processes are also provided.

Other embodiments provide a method of identifying antagonists or agonists of RGS activity, RGS binding to Gα, Gα binding to RGS or RGS/Gα complex activity by rational drug design comprising: (a) designing a potential antagonist or agonist that will form a reversible or non-reversible complex with one or more amino acids in the RGS Gα binding site based upon the structure coordinates of free RGS4; (b) synthesizing or otherwise obtaining the antagonist or agonist; and (c) determining whether the potential antagonist or agonist inhibits or promotes the activity or binding of RGS or the activity of the RGS-Gα complex. In other preferred embodiments, the antagonist or agonist is designed to interact with one or more atoms of one or more amino acids in the RGS4-Giα1 binding site. More specifically, the antagonist or agonist is designed to interact with amino acids selected from the group consisting of D117, S118, or R121 of RGS4, other amino acids associated with the Gα binding site and other amino acids revealed by the determined structure. Yet more specifically, the antagonist or agonist is designed to interact with amino acids selected from the group consisting of S39, E41, N42, L113, D117, S118, R121 or N82 of RGS4. Substances identified in accordance with such processes are also provided. The agonist or antagonist may form a covalent or non-covalent bond with an RGS protein. This method is specifically applicable to identifying antagonists or agonists of RGS subfamily B proteins.

Other specific embodiments provide a method of identifying antagonists or agonists of RGS activity or RGS/Gα complex activity by rational drug design comprising: (a) designing a potential antagonist or agonist that will form a reversible or non-reversible complex with one or more amino acids in a α6-α7 region of RGS based upon the structure co-ordinates of free RGS4; (b) synthesizing the antagonist or agonist; and (c) determining whether the potential antagonist or agonist inhibits or promotes the activity of RGS or RGS/Gα complex. In preferred embodiments, the antagonist or agonist is designed to prevent or facilitate conformation change in these regions on binding to Gα. This method is specifically applicable to identifying antagonists or agonists of RGS subfamily B protein activity or RGS subfamily B/Gα complex activity.

Other specific embodiments provide a method of identifying antagonists or agonists of RGS activity or RGS/Gα complex activity by rational drug design comprising: (a) designing a potential antagonist or agonist that will form a reversible or non-reversible complex with one or more amino acids in an RGS4 allosteric binding site based upon the structure co-ordinates of free RGS4; (b) synthesizing the antagonist or agonist; and (c) determining whether the potential antagonist or agonist inhibits or promotes the activity of RGS or RGS/Gα complex. In preferred embodiments, the antagonist or agonist is designed to interact with the allosteric binding site in the α₁-α₂ region of RGS4. In yet other preferred embodiments, the antagonist or agonist is designed to interact with one or more atoms of one or more amino acids in the allosteric binding site in the α₁ and α₂ region of RGS4, and particularly with one or more atoms of amino acids V10, W13, L17, L20, H23, E24, C25, or T132 of RGS4. Substances identified in accordance with such processes are also provided. This method is specifically applicable to identifying antagonists or agonists of RGS subclass B protein activity or RGS subclass B/Gα complex activity.

Candidate agonists and antagonists of RGS, RGS-Gα complexes can be selected from any type of small molecule, dimer, multimer, oligomer, or polymer of natural or non-natural origin that is obtained from any source and may be isolated from a natural source or chemically or biologically synthesized. Candidate antagonists and agonists can include nucleic acids, peptides, polypeptides, proteins, and various small organic molecules.

The study of the interaction of the candidate species with the three-dimensional structure of RGS and/or portions of that structure can be performed using available software platforms, including QUANTA, RASMOL, O, CHAIN, FRODO, INSIGHT, DOCK, MCSS/HOOK, CHARMM, LEAPFROG, CAVEAT(UC Berkley), CAVEAT(MSI), MODELLER, CATALYST, and ISIS.

The invention also provides a method for identifying the presence of and determining the location of allosteric binding sites in RGS4. The method comprises the steps of contacting free RGS4-core in solution with test compounds that are members of a library of chemical species which encompass a range of structural features or which are known to inhibit RGS function; measuring the ¹H, ¹⁵N, and/or ¹³C NMR spectra of the RGS4-core in the presence of test compounds of the library to detect any perturbations in the chemical shifts of RGS4-core that are induced by binding of a test compound to RGS4-core, and determining if binding of the test compound affects RGS activity. This can be done, for example, by assessing the affect of the test compound on RGS induced Gα GTPase activity. The amino acid residues of RGS4-core that are affected by binding of the test compound define the binding site of the test compound. If the test compound is found to affect RGS4-core activity and the location to which the test compound binds in RGS4-core is not the Gα binding site, then the location to which the test compound binds is an allosteric binding site. One such allosteric binding site in the α1-α2 region of RGS4-core has been identified using this method.

The three-dimensional structure of any allosteric binding site identified by this method can then be employed in methods described herein to identify, select and design candidate agonists and antagonists of RGS activity and specifically of RGS subclass B activity. Test compounds for assessing the presence of allosteric sites in RGS can be members of a library that exhibit a range of structural feature (e.g., alicyclic rings, heterocyclic rings, aromatic rings, aliphatic, alicyclic compounds or aromatic compounds displaying various substituent groups (e.g., OH,—CO—,—NHCO—, etc.). Test compounds can also be selected in screens for compounds that are known to exhibit an affect on RGS activity (e.g., that enhance or retard the rate of RGS4-induced Gα GTPase). Initial screens can be performed by assessing mixtures containing a plurality of test compounds for an affect on RGS activity. In cases in which an affect is observed with the mixture of test compounds, the individual compounds can be re-tested individually to determine which test compound(s) affect RGS activity.

In a specific embodiment, the invention provides a method in which the three dimensional structure of free RGS4-core is employed to identify chemical or biochemical species or fragments thereof capable of binding to an RGS protein. Once identified the species or fragments capable of binding to RGS are assembled (using well-known computer modeling techniques) into a single molecule to provide a structure of a potential antagonist or agonist. The molecule assembled can contain additional species or fragments (e.g., a backbone) for desired orientation of the species or fragments capable of binding to RGS. This method is particularly applicable to RGS subfamily B proteins.

The invention further provides a method for identifying mutants of RGS4 proteins in which the activity of the mutant protein is different from that of RGS4. In this method the three-dimensional structure of free RGS4 is employed to identify amino acids that are involved in the regulation of G-protein signaling. One or more of the amino acid residues identified are then modified to generate a mutant RGS4. Mutants identified in this method are expected to exhibit altered function in the regulation of G-protein signaling.

Other objects of the invention will be readily apparent from the following detailed description.

### BRIEF DESCRIPTION OF THE DRAWINGS

FIG. 1 illustrates the secondary structure of RGS4. The figure provides a summary of the sequential and medium range NOEs involving the NH, Hα and Hβ protons, the amide exchange and ³^{_{J}}HNα coupling constant data, and the ¹³Cα and ¹³Cβ secondary chemical shifts observed for RGS4 with the secondary structure deduced from this data. The thickness of the lines reflects the strength of the NOEs. Amide protons still present after exchange to D₂O are indicated by closed circles. The open boxes represent potential sequential assignments NOEs which are obscured by resonance overlap and could therefore not be assigned unambiguously. The gay boxes on the same line as the Hα(i)—NH(i+1) NOEs represents the sequential NOE between the Hα proton of residue i and the CδH proton of the i+1 proline and is indicative of a trans proline. Seven alpha helical regions are indicated (α1-α7).

FIGS. 2A and 2B are ribbon diagrams of the (A) x-ray structure of RGS4 from the RGS4-Giα1 complex, (B) NMR structure of free RGS4 for residues V5 to P134. The residues which exhibit a significant structural change between the RGS4-Giα1 x-ray structure and the free RGS4 NMR structure are numbered. Residues K116-Y119 correspond to key residues involved in the interaction with Giα1 and the location of a structural change between the free and complexed forms of RGS are indicated. The C- and N-terminal regions which exhibit a change in secondary structure and helical packing are also indicated. The RGS4-Giα1 x-ray structure is that of Tesmer et al. (8). The C- and N-terminal regions which incur a change in secondary structure and helical packing are indicated. The observed helical regions of the RGS4 structure are labeled.

### DETAILED DESCRIPTION OF THE INVENTION

RGS proteins are regulators of G-protein signaling which affect the intensity and duration of the G-protein signal cascade by binding to the active Gα-GTP-Mg² complex to increase the rate of GTP hydrolysis. RGS proteins act as attenuators of the induced G-protein signal by increasing the rate of inactivation of the G-protein and termination of the signal. RGS proteins have been identified in a wide range of eukaryotes, including humans. RGS proteins are highly diverse, multifunctional proteins characterized by the presence of a core region of approximately 130 amino acid residues (sometimes identified as having 120 amino acids), which may be separated by linker regions of varying lengths (79, 80, 9), that is conserved in all RGS proteins that have so far been identified. All RGS proteins that have been identified bind to members of the Giα class of G protein α subunits. The family of RGS proteins include RGS4, GAIP (human Gα-interacting protein), RGS 1, RGS 10, RGS 11, RGS12, RGS13, RGS14, and RGS16 (also called RGSr), Axin, Conductin, p115-RhoGEF, PD2-RhoGEF and LSC (86), among others, and contains more than 20 known members where specificity for Gα subtypes has been demonstrated and appears to be associated with subtle sequence differences (8, 14). RGS4 is believed to stabilize the transition state for GTP hydrolysis (17, 57, 21). The conserved region of RGS provides for binding to Gα and can thus be used to identify species that affect (as agonists or antagonists) RGS binding to Gα and the activity of RGS as an attenuator of G-protein signaling. RGS proteins of this invention function in G-protein regulation by binding to the Gα subunit of a G-protein. RGS proteins may, but need not, exhibit other biological functions. References 89 and 91 provided reviews of additional biological functions exhibited by RGS proteins.

The term “RGS protein” as used herein, including its use for specific RGS proteins and RGS protein subfamilies, includes native RGS proteins (and native RGS core proteins) isolated from or otherwise obtained from (e.g.,by expression of cloned genes) from any natural sources, recombinant RGS proteins which may contain portions of RGS sequence and non-RGS sequence (e.g., RGS-core sequence with the hexahis pro-tag), variant RGS proteins which contain conservative amino acid sequence differences from a native RGS protein or in which sequences non-functional in RGS activity are deleted, mutant RGS proteins in which one or more amino acids have been modified by expression from a mutant RGS coding sequence. Mutants include, among others, those having one or more site specific mutations, those having one or more deletions and those having one or more insertions compared to a native RGS protein (or RGS-core) or variant RGS (or variant RGS-core). The term mutant RGS refers in particular to those proteins having the described mutations, insertions or deletions in the RGS core region. Variant RGS proteins are expected to have biological function for G-protein regulation substantially the same as that of the native RGS protein from which they are derived. Mutant RGS proteins include those which have biological function substantially the same as or modified from that of a native or variant RGS protein from which they are derived. Variant, derivative, recombinant and mutant RGS proteins do not necessarily represent mutually exclusive subsets of proteins.

As noted herein, the RGS-core region is involved with RGS function in G-protein regulation, the term RGS proteins as used herein include RGS proteins in which non-functional regions are absent, e.g., RGS-core regions of native, recombinant, variant or mutant RGS proteins. The RGS core region of a native RGS has been found to retain full native RGS activity (8). The core region of RGS4 is approximately 130 amino acids in length. (References may also refer to conserved or core regions of RGS as having a length of approximately 120 amino acid) RGS cores from other RGS proteins can differ in length from that of RGS4. RGS proteins of this invention can be obtained by in vitro or in vivo expression of an RGS coding sequence by isolation from natural sources or any other means known in the art.

Known RGS proteins are categorized into six or seven subfamilies on the basis of a phylo-genetic analysis of 61 mammalian and invertebrate RGS proteins (86). Mammalian RGS proteins are composed of at least six subfamilies designated A-F as follows: A (GAIP, Ret-RGS1, RGS21); B (RGS1, RGS2, RGS3, RGS4, RGS5, RGS8, RGS13 and RGS16 [also called RGS-r]; C (RGS6, RGS7, RGS9 and RGS11); D (RGS12, RGS14); E (Axin and Conductin); and F (p115-RhoGEF, PD2-RhoGEF and Lsc). Two other RGS proteins TGS10 and D-AKAP2 are structurally diverse from those of subfamilies A-F and may represent a separate subfamily. Subfamilies B, C and D all have characteristic residue Asn (N82 in RGS4 as numbered herein, or N128 as numbered in Tesmer et al. [8]), which is associated with Gα binding at least in RGS subfamily B proteins. RGS proteins of subfamily A are substituted at this position in the RGS core with a serine (S156 in GAIP). Additionally, the B subfamily of RGS proteins is reported to have another characteristic residue, a serine (at position 57 in RGS4 as numbered herein and S103 as numbered in Tesmer et al. [8]). RGS4 represents the B subfamily of RGS proteins and is structurally more similar to and believed to have biological activity and function more similar to other members of the B subfamily, including RGS1, RGS2, RGS3, RGS5, RGS8, RGS13 and RGS16. GAIP, for example, is representative of the A subfamily of RGS proteins and is structurally more similar to and believed to have biological activity and function more similar to other members of the A subfamily including Ret-RGS1 and RGSZ1. Thus, the term RGS subfamily B refers to RGS1, RGS2, RGS3, RGS4, RGS5, RGS8, RGS13, RGS 16 and other, as yet uncharacterized, RGS proteins that exhibit structural features characteristic of the B subfamily and which are classifiable into the B subfamily by phylogenetic analysis as described in Zheng, B. et al. (1999) supra. Analogously, the term RGS subfamily A refers to GAIP, Ret-RGS1 and RGS21 and other RGS proteins as yet uncharacterized that exhibit structural characteristics of the A subfamily and which are classified as RGS subfamily A proteins by phylogenetic analysis. RGS subfamilies C-F have analogous definitions.

The term RGS4 refers to RGS4 exemplified by RGS4 of rat (Tesmer et al. (1997) supra) and homologs thereof including, among others, human RGS4 and mouse RGS4. The RGS core region of human, rat and mouse RGS4 differ from one another by 2-4 amino acids (representing about 97% or more sequence identify in the 130 amino acid core). Homologs of GAIP can exhibit as low as about 85% sequence identity in the RGS core region. An RGS4 homolog may, thus, exhibit RGS4 core sequence identify as low as about 85% with rat RGS4.

RGS protein NMR studies and structural determinations herein were performed using an RGS4-core protein consisting of the conserved region of RGS4 (specifically that derived from rat) with a N-terminal methionine and a C-terminal hexahistidine tail. The three-dimensional solution structure determined for the RGS4-core protein, assuming the possibility of conservative amino acids changes and within ± a root mean square deviation of the relative structural coordinates of the backbone atoms listed in Table 2 of not more than 1.5 Å (or more preferably, not more than 1.0 Å, or most preferably, not more than 0.5 Å), model the three-dimensional solution structures of other RGS4 proteins of any eukaryotic origin, including human RGS4. Further, because of the significant conservation of this domain among different RGS proteins, the three-dimensional structure of RGS4-core provided herein, again assuming conservative amino acids changes, and within ± a root mean square deviation of the relative structural coordinates of the backbone atoms of the structure of not more than 1.5 Å (or more preferably, not more than 1.0 Å, or most preferably, not more than 0.5 Å), models the structures of the conserved region in other RGS proteins of all origins.

“Structural coordinates” are the Cartesian coordinates corresponding to an atom's spatial relationship to other atoms in a molecule or molecular complex. Structural coordinates may be obtained using NMR techniques, as described herein or as known in the art, or using x-ray crystallography as is known in the art. Alternatively, structural coordinates can be derived using molecular replacement analysis or homology modeling. Various software programs allow for the graphical representation of a set of structural coordinates to obtain a three dimensional representation of a molecule or molecular complex. The structural coordinates of the present invention may be modified from the original sets provided in Table 2 by mathematical manipulation, such as by inversion or integer additions or subtractions. As such, it is recognized that the structural coordinates of the present invention are relative, and are in no way specifically limited by the actual x, y, z coordinates of Table 2. The structural coordinates of Table 2 ± a root mean square deviation from the conserved backbone atoms of the amino acids therein(or conservative substitutions thereof) of not more than 1.5 Å (or more preferably, not more than 1.0 Å, or most preferably, not more than 0.5 Å) define or embody the three-dimensional structure of free RGS4 (i.e., not complexed with another molecule) in solution. The RGS4 core conserved region contains a Gα binding site and an allosteric binding site. Amino acid sequences can be inserted between the helical regions of the RGS core region without significantly altering the biological function of the RGS protein. RGS proteins of lower eukaryotes contain such insertions.

“Root mean square deviation” is the square root of the arithmetic mean of the squares of the deviations from the mean, and is a way of expressing deviation or variation from the structural coordinates described herein.

As used herein, “RGS activity,” “activity of RGS” and other similar terms refer to the ability of RGS to bind to an active Gα-GTP-Mg² complex and induce a change in the rate of GTP hydrolysis. Any other biological function or activity of an individual RGS protein will be specifically defined herein. References 89 and 91 are incorporated by reference herein for their review of the additional biological functions of certain RGS proteins. Any assay which measures the rate of GTP hydrolysis in a Gα-GTP-Mg² complex in the presence and absence of RGS (or portions thereof) can be used to measure such activity. A preferred assay method measures precipitated radiolabeled phosphate that results from hydrolysis of Gα-[γ-³²P]-GTP-Mg² as described in the Examples herein.

Table 2 lists the atomic structure coordinates for the restrained minimized mean structure of free RSG4 as derived by NMR spectroscopy. The first two columns in Table 2 list atom number, the third column identifies the atom type using standard nomenclature, the fourth and fifth columns list the amino acid and its number in the sequence. The sixth, seventh and eighth columns of the table are relative coordinate values (in three dimensions).

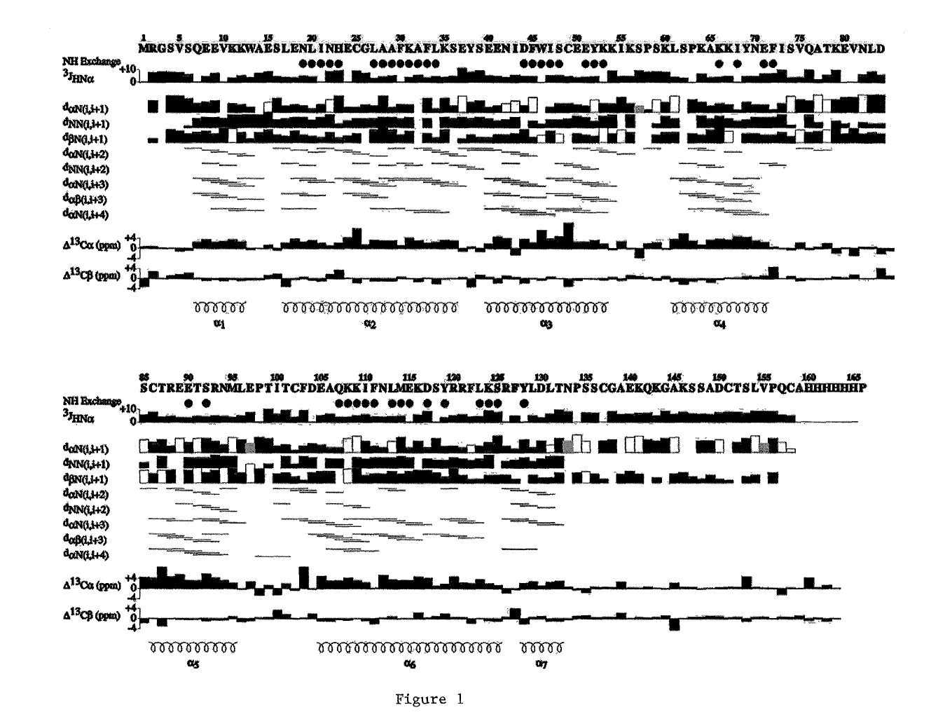

It will be obvious to the skilled practitioner that the numbering of the amino acid residues in the RGS4 and other RSG proteins covered by the present invention may be different than that set forth herein. The RGS4 core protein used herein contains an RGS core domain with an N-terminal Met and a six residue histidine tag at the C-terminus. In FIG. 1 the amino acid sequence of the RGS4 core protein used is numbered beginning at the N-terminal Met. For comparison to the full-length RGS4 sequence (for example, as numbered in Tesmer et al. (1997) (8)) add 46 to the numbering used herein.

It will also be obvious to the skilled practitioner that RGS proteins and portions thereof covered by this invention may contain certain conservative amino acid substitutions that yield the same three dimensional structures as those defined by the structural coordinates provided herein ± a root mean square deviation from the conserved backbone atoms of the amino acids therein(or conservative substitutions thereof) of not more than 1.5 Å. Amino acids in other RGS proteins or peptides corresponding to those in RGS4 and conservative substitutions in other RGS proteins or peptides are readily identified by visual inspection of the relevant amino acid sequences or by using commercially available homology software programs. “Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three dimensional structure of RGS with respect to the use of said structure for the identification and design of RGS antagonists or agonists, and for molecular replacement analyses and/or for homology modeling.

The structural coordinates of the present invention permit the use of various molecular design and analysis techniques in order to (i) solve the three dimensional structures of related RGS proteins, peptides or complexes thereof, and particularly RGS subfamily B proteins, peptides or complexes thereof and (ii) to select, design and synthesize or otherwise obtain chemical and biochemical species capable of associating, binding or interacting with RGS potentially having function as antagonists or agonists of an RGS, Gα or an RGS-Gα complex.

Molecular replacement analysis is a well-known technique employed in x-ray crystallography which uses the x-ray structure of a molecule having as a starting point to model a molecule whose crystal structure is unknown. This technique is based on the principle that two molecules which have similar structures, orientations and positions will diffract x-rays similarly. A corresponding approach to molecular replacement is applicable to modeling an unknown solution structure using NMR technology. The NMR spectra and resulting analysis of the NMR data for two similar structures will be essentially identical for regions of the molecules that are structurally conserved, where the NMR analysis consists of obtaining the NMR resonance assignments and the structural constraint assignments, which may contain hydrogen bond, distance, dihedral angle, coupling constant, chemical shift and dipolar coupling constant constraints. Appropriate NMR spectra are accumulated for a solution of the species of unknown structure and compared to NMR of the species of known structure. The observed differences in the NMR spectra of the two structures will highlight the differences (and similarities) between the two structures and identify the corresponding differences in the structural constraints. The structure determination process for the unknown structure is then based on modifying the NMR constraints from the known structure to be consistent with the observed spectral differences. This method is applicable to the determination of three-dimensional solution structures of any RGS protein or peptide using the structural information for RGS4 provided herein. The method is most appropriate for determining the structures of RGS proteins that are expected to have significant structural similarity with RGS4. For example, this invention specifically provides the three-dimensional structure of a rat RGS4-core region in solution. The replacement method described above can be employed to determine the three-dimensional structure of the human RGS4-core which differs from that of rat by 2 amino acids in the 130 RGS core (at positions 22 N (rat)>S(human), and 132 T(rat)>V(human), referring to the rat sequence given in FIG. 1.

Accordingly, in one nonlimiting embodiment of the invention, the NMR resonance assignments for RGS4 provide the starting point for resonance assignments of other RGS family proteins (or portions thereof), that are expected to be structurally similar to RGS4, e.g., RGS4 homologs from different organisms or more generally RGS proteins of the subfamily B. Chemical shift perturbations can be detected using one or two dimensional spectra (e.g.,¹⁵N/¹H, ¹³C/¹H spectra) or using other methods well known in the art and compared between RGS4 and another RGS protein. In this way, the affected residues may be correlated with the three dimensional structure of RGS4 as provided by the relevant residues of Table 2. This effectively identifies the region of the other RGS protein or peptide that has a structural change relative to the RGS4 protein. The ¹H, ¹⁵N, ¹³C and ¹³CO NMR resonance assignments corresponding to both the sequential backbone and side-chain amino acid assignments of the other RGS protein, or portion thereof, can then be obtained and the three dimensional structure of this protein, or portion thereof, can be generated using standard 2D, 3D and 4D triple resonance NMR techniques and NMR assignment methodology, using the RGS4 structure, resonance assignments and structural constraints as a reference. Various computer fitting analyses of the other RGS protein or peptide with the three dimensional model of RGS4 can be performed in order to generate an initial three dimensional model of the other RGS protein or peptide, and the resulting three dimensional model may be refined using standard experimental constraints and energy minimization techniques in order to position and orient the other RGS in association with the three dimensional structure of RGS4.

The present invention further provides that the structural coordinates of the present invention can be used with standard homology modeling techniques in order to determine the unknown three-dimensional structure of an RGS protein or portion thereof. Homology modeling, as is known in the art, involves constructing a model of an unknown structure using structural coordinates of one or more related protein molecules, molecular complexes or parts thereof (i.e., active sites). Homology modeling may be conducted by fitting common or homologous portions of the protein whose three dimensional structure is to be solved to the three dimensional structure of homologous structural elements in the molecule of known three-dimensional structure, specifically using the relevant (i.e., homologous) structural coordinates provided by Table 2. Homology can be determined a variety of known methods, for example, using amino acid sequence identity, homologous secondary structure elements, and/or homologous tertiary folds. Tesmer et al. (1997) (8) and Druey and Kehrl (1997) (88) provide examples of multiple sequence alignments of RSG protein sequences. Homology modeling can include rebuilding part or all of a three dimensional structure with replacement of amino acids (or other components) by those of the related structure to be solved. Molecular replacement analysis as adapted and applied to NMR structural data (as discussed above) and homology modeling are techniques that are well known in the art which can be readily applied or adapted to the determination of the three dimensional structures of other proteins of the RGS family (and portions thereof, e.g., Gα binding sites and/or allosteric binding sites). These methods are particularly useful for determining RGS solution structure within the conserved region of the protein based on the RGS4 three-dimensional solution structure. These methods are applicable to presently known members of the RGS family of proteins as well as to proteins, particularly those of RGS sub-family B, as yet unidentified as RGS proteins, and particularly those that exhibit significant sequence identity above 60% or more, preferably 85% or more sequence identity in the RGS-core region. NMR assignments, structural coordinates and three-dimensional structures of RGS proteins or peptides, determined using molecular replacement analysis and homology modeling based on the structural coordinated and NMR assignments provided herein and optionally refined using a number of techniques well known in the art, can be employed in a similar fashion to the structural coordinates of Table 2 for identifying, selecting or designing chemical species that are antagonists or agonists of RGS, Gα or RGS Gα complexes.

Description of the Structure of RGS4

The primary amino acid sequence of several RGS4 proteins are known. The amino acid sequence of RGS4-core (from rat) with attached hexahis pro tail is listed in FIG. 1 (as SEQ ID No. 1). The regular secondary structure elements of free RGS4 were identified from a qualitative analysis of sequential and inter-strand NOEs, NH exchange rates, ³J_{HNα} coupling constants and the ¹³Cα and ¹³Cβ secondary chemical shifts (47, 48). The sequential and medium NOEs were obtained from a qualitative analysis of the ¹⁵N-edited NOESY and ¹³C-edited NOESY spectra. ³J_{HNα} coupling constants were obtained from the relative intensity of Hα crosspeaks to the NH diagonal in the HNHA experiment (18). Slowly exchanging NH protons were identified by recording an HSQC spectra two hours after exchanging an RGS4 sample from H₂O to D₂O. These data, together with the deduced secondary structure elements are summarized in FIG. 1.

The overall structure of RGS4 is composed of seven helical regions corresponding to residues 7-12 (α₁); 17-36 (α₂); 40-53 (α₃); 61-71 (α₄); 86-95 (α₅); 105-125 (α₆) and 128-132 (α₇). A simple description of the RGS4 topology is that the protein consists of two pseudo 4-helix bundles with an up-down-up-down arrangement where helical region six is part of both bundles. An unusual feature of the RGS4 structure occurs in the second helical region. There is a one residue (H23) ˜90° bend in the helix which effectively divides this helical region into two separate helices (as described in the RGS4 x-ray structure (5)). This one residue bend was not obvious from the NMR analysis of the secondary structure data (FIG. 1) where it appears to be a continual helical stretch. The bend only became apparent during the structure refinement process. Some observable NOEs that contribute to the bend at H23 occur between residues L20, I21, and residues G26, L27,A29 and F30. The bend at H23 effectively allows for appropriate packing of these hydrophobic side-chains.

Additional bends or turns occur throughout the RGS4 structure. Helical regions α₁ and α₂ are connected by residue S16 that adopts an extended conformation allowing these two helices to be essentially parallel. This is very similar to the turns connecting helical regions α₃ and α₄ and helical regions α₅ and α₆. Conversely, helical regions α₂ and α₃ are connected by Y38 that has a positive Φ torsion angle, suggesting a β type turn. The conformation of Y38 results in an angle between helical regions α₂ and α₃ of ˜45°, which also represents a transition point between the two pseudo 4-helix bundles. The longest loop in the structure occurs between helical regions α₄ and α₅. This loop region is well ordered based on high order parameters (S²>0.6). The low mobility for this loop results from interactions with helical regions α₃ and α₆. The observed bend between the longest helical region α₆ and the shortest helical region α₇ is suggestive of a distortion in this helical segment to achieve an optimal packing interaction between helical regions α₁ and α₇. The end result of these local conformations on the overall topology of RGS4 is to create an elongated structure where the two pseudo 4-helix bundles are nearly perpendicular. The interface between these the two pseudo 4-helix bundles is predominately hydrophobic in nature (L17, I21, L27, F30, L34, W46, I47, I110, F111, L113, M114) consistent with the general packing of hydrophobic residues in the core of the protein with charged residues on the protein surface.

As previously described, the primary biological function for RGS4 is to bind G_{iα1} and stimulate its intrinsic GTPase activity. Key residues in the RGS4 structure that are involved in the interaction of RGS4 with G_{iα1} correspond to RGS4 residues S39, E41, N42, L113, D117, S118, and R121 that form the binding pocket for T182 from G_{iα1}. Similarly, N82 from RGS4 binds into the G_{iα1} active site interacting with residues Q204, S206 and E207 (8) of G_{iα}. RGS4 mutational work support the functional importance of these residues in the binding and activity of RGS4 with G_{iα1} while identifying N82 to be critical in facilitating GTP hydrolysis (18-20). RGS4 residues S39, E41 and N42 are located in the N-terminal end of helical region α₃ while L113, D117, S118, and R121 are located directly opposite at the C-terminal end of helical region α₆. N82 is located approximately in the center of the structured loop region between helical regions α₄ and α₅ which is positioned relatively above the T182 binding pocket on RGS4.

Another feature of the RGS4 structure is the observation that residues M1-S4 and P134-H166 are completely disordered and dynamically flexible. Structure coordinates for these atoms are not included in Table 2. This is evident by the sharp line-widths and the minimal number of observable NOEs. The flexible nature of these residues are further supported by ¹⁵N T1, T2 and NOE measurements which indicate low order-parameters (S²<0.6)

RGS4 Structure Determination

The final 30 simulated annealing structures were calculated on the basis of 2871 experimental NMR restraints consisting of 1960 approximate interproton distance restraints, 78 distance restraints for 39 backbone hydrogen bonds, 431 torsion angle restraints comprised of 151 Φ, 154 ψ, 97_{χ1}, and 29_{χ2} torsion angle restraints, 132 ³J_{NHα} restraints and 136 Cα and 134 Cβ chemical shift restraints. Stereospecific assignments were obtained for 58 of the 125 residues with β-methylene protons, for the methyl groups of 3 of the 5 Val residues, and for the methyl groups of 9 of the 12 Leu residues. In addition, 7 out of the 8 Phe residues and 4 out of the 5 Tyr residues were well defined making it possible to assign NOE restraints to only one of the pair of CδH and CεH protons and to assign a _{χ2} torsion angle restraint.

Comparison of the Free RGS4 NMR Stricture with the RGS4 G_{iα1} Bound Structure

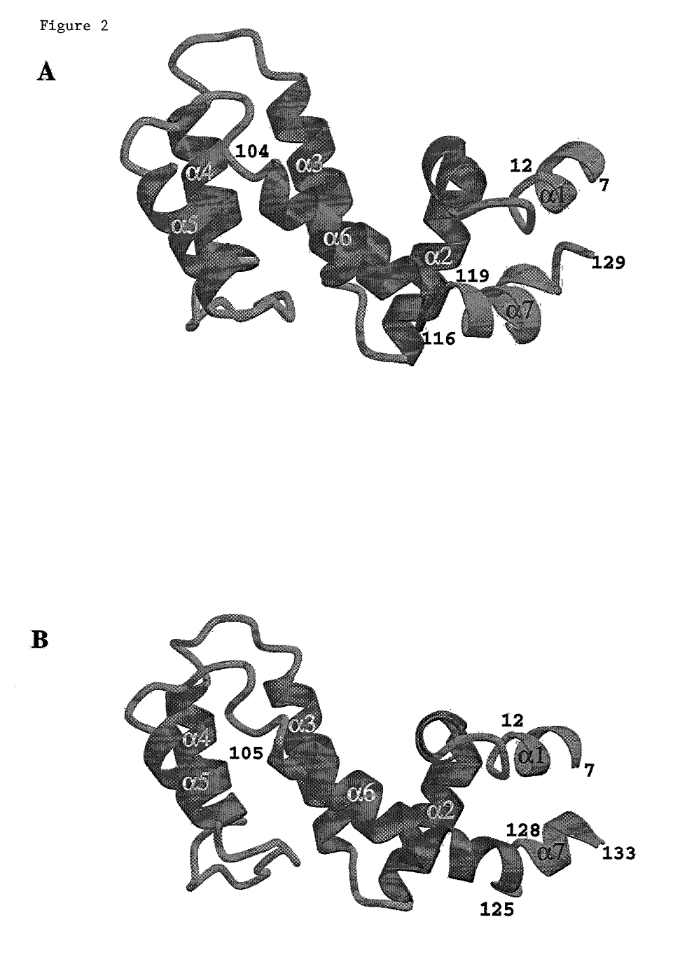

FIGS. 2A and 2B are ribbon diagrams of (A) the x-ray structure of RGS4 complexed to Giα1 (8) and (B) the solution structure of RGS4 as determined by NMR methods. Residues that effect significant structural change between the two structures are indicated. An unexpected result from determining the solution structure of RGS4 in the absence of G_{iα1} was the observation of a significant change in the conformation for free RGS4 relative to RGS4 in the complex (5). A fundamental factor in the difference between the two structures is a perturbation in the secondary structure elements. Consistent with the RGS4-G_{iα1} x-ray structure, the NMR structure of free RGS4 is an a-helical protein comprised of two peudo 4-helix bundles. The NMR data shows that free RGS4 is composed of seven helical regions and a majority of this data is consistent with the RGS4-G_{iα1} x-ray structure. The significant difference between the two secondary structures occurs within the C-terminal helical regions α₆ and α₇. In the RGS4-G_{iα1} x-ray structure residues V5 to T132 are observed in (i.e., they are ordered) and residues 104-116 and 119-129 are helical. This contrasts with the free RGS4 NMR structure where residues 5-133, 105-125 (α₆) and 128-132 (α₇) are helical. There is a significant shift in the helical structure in this region containing residues 104-133.

The observed structural change between the free RGS4 NMR structure and the RGS4-G_{iα1} x-ray structure is a movement of a kink between helical regions α₆ and α₇ towards the C-terminus. The movement of this kink results in α₆ being longer by nine residues and α₇ being shorter by six residues in the free RGS4 NMR structure. Additionally, α₇ of free RGS4 extends three residues beyond what was observed as a structural region in the RGS4-G_{iα1} x-ray structure.

The observed change in the secondary structure, although only involving a few C-terminal residues, has far-reaching effect, since it results in a significant modification in the overall fold for RGS4. This is evident from a 1.94 Å backbone rms difference between the RGS4-G_{iα1} x-ray structure and the free RGS4 NMR structure for residues 5-134. The major effect of the alteration in secondary structures is a reorganization of the packing of the N-terminal and C-terminal helix as is evident from per-residue backbone atomic rms differences between the free RGS4 NMR structure and the bound RGS4 x-ray structure. Therefore, the accuracy of the secondary structure interpretation is important for proper analysis of the free RGS4 structure. The reliability of the NMR secondary structure is demonstrated by the extensive data summarized in FIG. 1. Residues 105-125 and 128-132 show a continual stretch of NMR data consistent with an α-helical definition with an abrupt break in this information for residues 125-128. Furthermore, the significant differences between the - and C-terminal regions of the free RGS4 NMR structure and the bound RGS4 x-ray structure are indicated by a large number of interproton distance (145) and torsion angle (39) violations and by the corresponding very high values for the NOE and torsion angle restraint energies exhibited by the bound RGS4 x-ray structure. The self-consistency of the NMR data using NOEs, coupling constants, NH exchange rates and secondary carbon chemical shifts and the large number of restraint violations with the bound structure, demonstrate the accuracy of the RGS4 NMR structure provided. Comparisons of the free RGS4 structure of this invention with the structure of the RGS4-Giα1 complex should then provide an accurate description of the conformational changes that occur in on RGS4 on binding to Giα1.

Relevance to Activity for the RGS4 Conformational Change

RGS4 is involved in the regulation of the G_{iα1} GTPase cycle having a modest affinity for GTP-Gα, but not binding to GDP-Gα. It is believed that the observed conformational chances for free RGS4 are related to modulating its affinity to G_{iα1} to allow for perpetuation of the GTPase cycle. This role for the RGS4 conformational change is evident by the fact that the RGS4 Gα binding site is the location of the G_{iα1} induced structural perturbation. The pronounced kink between helical regions α₆ and α₇ observed in the bound RGS4 x-ray structure occurs at residues D117 and S118. RGS4 molecular surfaces for both the free RGS4 NMR structure and the RGS4-G_{iα1} x-ray structure in the vicinity of the G_{iα1} T182 binding pocket were calculated. A comparison of the two RGS4 molecular surfaces, shows that the G_{iα1} T182 binding pocket is larger and more accessible in the free RGS4 NMR structure. Also, in the RGS4-G_{iα1} x-ray structure there appears to be a molecular surface “wall” composed of the RGS4 sidechains from residues D117, S118 and R121 which surround the G_{iα1} T182 binding pocket. These residues form an important hydrogen-bonding network which is critical for the binding of RGS4 with G_{iα1} where D117 forms a hydrogen bond with R121 and the backbone nitrogen of G_{iα1} T182. The critical nature of these residues is further supported by mutagenesis. Alanine mutations of D117 and R121 diminishes RGS4 activity and binding to G_{iα1}. Since the helical kink at residues D117 and S118 is less pronounced in the free RGS4 NMR structure and a disruption in the helix occurs instead between residues 125-128, the sidechains for D117, S118 and R121 are well beyond hydrogen-bonding distance. It is evident from the free RGS4 NMR structure that the network of sidechain interactions with G_{iα1} T182 in the absence of G_{iα1} is not pre-formed.

The observation that RGS4 undergoes a significant structural change in the presence of G_{iα1} where the focal point of this change occurs at key residues in the RGS4-G_{iα1} interface creates a different explanation for the process of RGS4 activation of G_{iα1} GTPase activity. This information suggests a two-stage process composed of a binding and locking step. Because the G_{iα1} T182 binding pocket is clearly more accessible in the free RGS4 NMR structure, the binding step appears to be driven by the fit of T182 into this pocket. The locking step then results from the induced conformational change in the RGS4 structure where the pronounced kink in the helix between residues D117 and S118 brings these residues into close contact with R121 and G_{iα1} T182 to form the hydrogen bonding network observed in the RGS4-G_{iα1} x-ray structure. T182 in the binding pocket induces the formation of a hydrogen bonding network and the resulting RGS4 conformational change as opposed to a pre-formed binding site suggested from the RGS4-G_{iα1} x-ray structure. The release of RGS4 from G_{iα1} would then require the removal of G_{iα1} T182 from the RGS4 binding pocket which presumably occurs during GTP hydrolysis. This mechanism is consistent with a local perturbation in the vicinity of T182 seen between the GDP-G_{iα1} (2)and RGS4-G_{iα1} x-ray structures where this localized movement appears to be sufficient to remove T182 from the RGS4 binding site and disrupt the hydrogen-bonding network resulting in dissociation of the complex. Comparison of the T182 G_{iα1} region between the RGS4-G_{iα1} and the GDP-AlF₄-G_{iα1} x-ray structures (4) indicate that these two structures are essentially identical in this region of G_{iα1}. Since the GDP-AlF₄-G_{iα1} structure corresponds to the active form of GDP-AlF₄-G_{iα1} as well as the conformation that RGS4 preferentially binds, the similarity between these two structures is also consistent with the proposed mechanism for the activity of RGS4.

The x-ray structure of RGS4 complexed with G_{iα1} in conjunction with other Gα conformers suggest that the role of RGS4 in stimulating Gα GTPase activity is accomplished by stabilizing the GTP hydrolysis transition state. The NMR structure of free RGS4 reported here expands this mechanism suggesting that the RGS4 induced conformation in the presence of G_{iα1} maybe related to its GTP-G_{iα1} specificity which facilitates binding turnover that is critical for perpetuating the GTPase cycle. The described structural change in RGS4 provides an elegant mechanism for the observed binding selectivity between the various Gα conformers despite the close similarity in these structures.

Detection of an Allosteric Binding Site in RGS4

Several small molecule inhibitors of the RGS4-Gα interaction were identified in a large scale screening based on detection of inhibition of Gα GTPase function which implies inhibition of the binding of RGS4 to Gα. One of these compounds (designated compound 1 for convenience) exhibited 100% inhibition of binding. The nature of the activity of compound 1 and its ability to inhibit RGS4 binding to Gα was further investigated by ¹H-¹⁵N HSQC chemical shift perturbation experiments. A total of five compounds, three that had exhibited inhibition of RGS4 binding in the screen and two controls that showed no activity in the screen were examined. 2D ¹H-¹⁵N HSQC spectra were collected for a ¹⁵N-enriched RGS4 sample and a series of ¹⁵N-enriched RGS4 samples titrated with one of the three test compounds and two controls. Comparison of the HSQC spectra of a free RGS4 sample and each of the samples titrated with a potential inhibitor allowed the identification of any chemical shift changes for RGS4 in the presence of the test and control compounds. In such an analysis the observation of a change in the position shape or intensity of a resonance indicates perturbation. With the NMR instrumentation employed a shift of half a line width in peak position could be reliably detected. Only in NMR spectra taken of RGS4 in the presence of compound 1 were any chemical shift perturbations observed indicating that compound 1 directly binds to RGS4. Employing the chemical shift assignments for free RGS4 (Table 1), the binding site of compound 1 in RGS4 was identified. RGS4 amino acid residues V10, W13, 117, 120, H23, E24, C25 and T125 exhibited a chemical shift perturbation in the presence of compound 1.

The observed chemical shift perturbations did not arise from a pH change caused by addition of compound 1 to the RGS4 solution. (¹H-¹⁵N HSQC spectra of free RGS4 taken over a pH range of 5.5-6.5 indicate that none of the amino acid residues listed above was sensitive to pH changes over this range). The binding site for compound 1 corresponds to residues in the α1-α2 region of RGS4 (where the α1-α2 region includes the tight turn between the two helices). In the three-dimensional structure of RGS4, the binding region is positioned on the opposite surface from the Gα binding site. No amino acids residues associated with the Gα binding site exhibited any chemical shift perturbation in the presence of compound 1. This indicates that the structure of the RGS4 Gα binding site is unchanged in the presence of compound 1. Compound 1 was found to significantly decrease the expected GTPase activity of Gα which combined with the fact that compound 1 binds at a site distal from the Gα binding site indicates that compound 1 is an allosteric inhibitor of RGS4 and that there is an allosteric binding site in the α1-α2 region of RGS4. It is believed that binding of compound 1 at the allosteric binding site stabilizes RGS4 in the free form and effectively locks the RGS4 protein in free form. Compound 1 prevents the formation of a hydrogen-bonding network around the Gα T182 binding pocket.

The solution structural information provided herein, including the secondary and tertiary structure of RGS4-core, the RGS4 Gα binding site, the α6-α7 region, and the allosteric binding site in the α1-α2 region of RGS4 can all be employed in methods described herein and methods well known in the art to identify, select or design candidate agonists and antagonists of RGS4 activity which in turn affects G-protein signaling functions in various eukaryotic cells and organisms.

The following examples are provided to further illustrate the invention and are not intended to limit the invention.

### EXAMPLES

The following abbreviations are used herein:

G-proteins, heterotrimeric guanine nucleotide-binding proteins; RGS4, Regulators of G-protein Signaling; G_{iα1}, Gα subunit of heterotrimeric G proteins, G_{iα1}-AIF₄⁻, Gα subunit of heterotrimeric G proteins complexed with Mg²⁺, GDP and AlF₄⁻ stabilized in the transition state for GTP hydrolysis, DTT, DL-1,4-Dithiothreitol; GTP, guanosine triphosphate; GDP, guanosine diphosphate; NMR, nuclear magnetic resonance; 2D, two-dimensional; 3D, three-dimensional; HSQC, heteronuclar single-quantum coherence spectroscopy; HMQC, heteronuclear multiple-quantum coherence spectroscopy; TPPI, time-proportional phase incrementation; NOE, nuclear Overhauser effect; NOESY, nuclear Overhauser enhanced spectroscopy; COSY, correlated spectroscopy; HNHA, amide proton to nitrogen to CαH proton correlation; HNHB, amide proton to nitrogen to CβH proton correlation; CT-HCACO, constant time CαH proton to α-carbon to carbonyl correlation; HACAHB, CαH proton to α-carbon to CβH proton correlation.

### Example 1

### Assignment of NMR Peaks and Secondary Structure Determination

The RGS core domain of RGS4 was expressed in Escherichia coli (J109) using the prokaryotic expression vector pQE50 (Qiagin, Valencia, Calif.). PCR was used to amplify and add a C-terminal hexahis -pro tag to the RGS core (here residues 51-206 of RGS4) and the product was ligated between the BamH1 and Sal1 sites of pQE50 to give plasmid pRGS4. E. coli (BL21(DE3)) containing pRGS4 were grown in LB broth supplemented with 100 μg/mL ampicillin. An overnight culture was diluted 1:20 and grown at 37° C. to an A₆₀₀ of 0.6 -0.8 with vigorous shaking. Isopropyl β-D-galactoside (1PTG) was added to a final concentration of 1 mM and cultures were shaken for 3 h at 37° C. The cells were harvested by centrifugation (7000×g) for 15 min. at 4° C., washed with PBS and stored at −70° C.

Uniformly (>95%) ¹⁵- and ¹³C-labeled recombinant RGS4-core (containing the 166 amino acid core domain of RGS4 with an N-terminal methionine and C-terminal hexahis-pro tag) was obtained by growing BL21 (DE3) E. coli in defined medium containing 2.0 g/L [¹³C6, 98%+] D-glucose and 1.0 g/L [¹⁵N,98%+] ammonium chloride as sole carbon and nitrogen sources, respectively. In addition, the defined medium contained M9 salts, trace elements, vitamins and 100 μg/L ampicillin. Conditions for induction and growth are as described above. The recombinant RGS4-core protein was purified using affinity chromatography on a 10 mL Ni²⁺ column and purified to homogeneity following ion-exchange chromatography on Resource S at pH 5.5. Protein was desalted into appropriate buffer prior to use. N-terminal amino acid sequencing was performed to confirm protein identity and uniform labeling of RGS4-core was confirmed by MALDI-TO mass spectrometry (Perceptive Biosystems).

The NMR samples contained 1 mM of RGS4 manually purified-core protein in a buffer containing 50 mM K₂PO₄, 2 mM NaN₃, and 50 mM deuterated DTT, in either 90% H₂O/10% D₂O or 100% D₂O at pH 6.0.

All spectra were recorded at 30-35° C. on a Brucker AMX-2 600 spectrometer using a gradient enhanced triple-resonance ¹H/¹³C/¹⁵N probe. For spectra recorded in H₂O, water suppression was achieved with the WATERGATE sequence and water-flip back pulses (23, 24). Quadrature detection in the indirectly detected dimensions were recorded with States-TPPI hypercomplex phase increment (25). Spectra were collected with appropriate refocusing delays to allow for 0,0 or −90,180 phase correction. Spectra were processed using the NMRPipe software package (28) and analyzed with PIPP (29), NMR Pipe and in a peak sorting program—on a Sun Ultra10 Workstation. When appropriate, data processing included a solvent filter, zero-padding data to a power of two, linear predicting back one data point of indirectly acquired data to obtain zero phase corrections, linear prediction of additional points for the indirectly acquired dimensions to increase resolution. linear prediction by the means of the mirror image technique was used only for constant-time experiments (38) In all cases data was processed with a skewed sine-bell apodization function and one zero-filling was used in all dimensions.

The assignments of the ¹H, ¹⁵N, ¹³CO, and ¹³C resonances were based on the following experiments: CBCA(CO)NH (62), CBCANH (63), C(CO)NH (64), HC(CO)NH (64), HBHA(CO)NH (65), HNCO (66), HCACO (29), HNHA (26), HNCA (67), HCCH-COSY (68) and HCCH-TOCSY (69) (for reviews see: Bax et al 1994 and Clore and Gronenborm, 1994). The resonance assignments of RGS4 essentially followed the semi-automated protocol described previously (37, 70, 71). The accuracy of RGS4-core assignment was further confirmed by sequential NOEs in the ¹⁵N-edited NOESY-HMQC spectra. Because the RGS4 structure is exclusively α-helical, the sequential NH₁—NHᵢ₊ₗ NOEs were extremely useful in completing the RGS4 backbone assignments. ¹H, ¹⁵N, ¹³C AND ¹³CO assignments for RGS4-core are summarized in Table 1.

The backbone ¹H, ¹⁵N, ¹³CO, and ¹³C assignments in Table 1 are essentially complete for the RGS4-core As noted above, the native core sequence was appended to six histidines. The last five histidines were the only unassigned residues in the protein. The ability to obtain the complete assignments for RGS4-core implies a well-packed ordered structure. The side-chain assignments are also nearly complete; the majority of missing information is in residues with long side-chains which are potentially solvent exposed.

The secondary structure of the RGS4-core (summarized in FIG. 1) is based on characteristic NOE data involving the NH, Hα and Hβ protons from ¹⁵N-edited NOESY-HMQC and ¹³C-edited NOESY-HMQC spectra, ^{3J}HNα coupling constants from HNHA, slowly exchanging NH protons and ¹³Cα and ¹³Cβ secondary chemical shifts (for reviews see: (56) and (78). It was determined that the RGS4-core solution NMR was composed of seven helical regions corresponding to residues 7-12(α1);17-36(α2); 40-53(α3); 61-71(α4); 86-95 (α5); 105-125 (α6); and 128-132 (α7). The RGS4-core overall fold is essentially comprised of two 4-helix bundles with the long helical region α6 part of both bundles. A distinct difference in the RGS4-core secondary structure in solution from the x-ray structure of the RGS4-Giα1 complex was unexpectedly observed at the C-terminus. The x-ray structure indicates that residues 104-116 and 119-129 are helical where only residues V5 to T132 are observed. The solution NMR structure indicates that residues 105-125 and 128-132 are helical and residues P134-H166 appear, in view of the sharp line-widths observed, to be extremely mobile. The differences in secondary structure between the x-ray crystal structure and that of free RGS4-core suggest a conformational change in RGS4 on binding to Gα.

### Example 2

### Three-Dimensional Structure Determination for RGS4-Core

RGS4-core was prepared, purified and uniformly labeled as in Example 1. NMR samples were prepared and spectral data accumulated as indicated in Example 1.

The RGS4 structure is based on the following series of spectra: HNHA (26), HNHB (27), 3D long-range ¹³C-¹³C correlation (28), coupled CT-HCACO (29, 30), HACAHB-COSY (31), 3D ¹⁵N- (32, 33) and ¹³C-edited NOESY (35, 37) experiments. The ¹⁵N-edited NOESY, and ¹³C-edited NOESY experiments were collected with 100 msec and 120 msec and mixing times, respectively.

Spectra were processed using the NMRPipe software package (36) and analyzed with PIPP (37) on a Sun Ultra10 Workstation. When appropriate, data processing included a solvent filter, zero-padding data to a power of two, linear predicting back one data point of indirectly acquired data to obtain zero phase corrections, linear prediction of additional points for the indirectly acquired dimensions to increase resolution. Linear prediction by the means of the mirror image technique was used only for constant-time experiments (38). In all cases, data were processed with a skewed sine-bell apodization function and one zero-filling was used in all dimensions.

Interproton Distance Restraints

The NOEs assigned from 3D ¹³C-edited NOESY and 3D ¹⁵N-edited NOESY experiments were classified into strong, medium, weak and very weak corresponding to interproton distance restraints of 1.8-2.7 Å (1.8-2.9 Å for NOEs involving NH protons), 1.8-3.3 Å (1.8-3.5 Å for NOEs involving NH protons), 1.8-5.0 Å, and 3.0-6.0 Å, respectively (39, 40). Upper distance limits for distances involving methyl protons and non-stereospecifically assigned methylene protons were corrected appropriately for center averaging (41).

Torsion Angle Restraints and Stereospecific Assignments

The β-methylene stereospecific assignments and _{χ1} torsion angle restraints were obtained primarily from a qualitative estimate of the magnitude of ³J_{αβ} coupling constants from the HACAHB-COSY experiment (31) and ³J_{Nβ} coupling constants from the HNHB experiment (27). Further support for the assignments was obtained from approximate distance restraints for intraresidue NOEs involving NH, CαH, and CβH protons (42).

Theφ and ψ torsion angle restraints were obtained from ³J_{NHα} coupling constants measured from the relative intensity of Hα crosspeaks to the NH diagonal in the HNHA experiment (26), from chemical shift analysis using the TALOS program (43) and from consistency with distance restraints for intraresidue and sequential NOEs involving NH, CαH, and CβH protons. ¹J_{CαHα} coupling constants obtained from a coupled 3D CT-HCACO spectrum were used to ascertain the presence of non-glycine residues with positive φ backbone torsion angles (30). The presence of a ¹J_{CαHα} coupling constant greater then 130 Hz allowed for a minimum φ restraint of −20 to −178°.

The Ile and Leu _{χ2} torsion angle restraints and the stereospecific assignments for leucine methyl groups were determined from ³J_{CαCδ} coupling constants obtained from the relative intensity of Cα and Cδ cross peaks in a 3D long-range ¹³C-¹³C NMR correlation spectrum (44), in conjunction with the relative intensities of intraresidue NOEs (45). Stereospecific assignments for valine methyl groups were determined based on the relative intensity of intraresidue NH—CγH and CαH—CγH NOEs as described by Zuiderweg et al. (1985) (46). The minimum ranges employed for the φ, ψ, and χ torsion angle restraints were ±30°,±50°, and ±20° respectively (47)

Structure Calculations

The structures were calculated using the hybrid distance geometry-dynamical simulated annealing method of Niles et al. (1988) (48) with minor modifications (49) using the program XPLOR (50), adapted to incorporate pseudopotentials for ³J_{NHα} coupling constants (51), secondary ¹³Cα/¹³Cβ chemical shift restraints (52) and a conformational database potential (53, 54). The target function that is minimized during restrained minimization and simulated annealing comprises only quadratic harmonic terms for covalent geometry, ³J_{NHα} coupling constants and secondary ¹³Cα/¹³Cα chemical shift restraints, square-well quadratic potentials for the experimental distance and torsion angle restraints, and a quartic van der Waals term for non-bonded contacts. All peptide bonds were constrained to be planar and trans. There were no hydrogen-bonding, electrostatic, or 6-12 Lennard-Jones empirical potential energy terms in the target function.

Analysis of a T-182 Binding Site on RGS4-Core

The overall appearance of the NMR structure in the area of the proposed T182 (of Gα) binding site is one of great interest. To obtain a more quantitative measurement of the differences in accessibility between the free RGS4 NMR structure and the x-ray structure of the RGS4-G_{iα1} complex, MOLCAD (commercially available from TRIPOST) surfaces were calculated for both structures and the surface area of each was measured.

The x-ray structure of the RGS4-G_{iα1} complex (AGR1) was read into SYBYL (Tripos) and all substructures except chain E (RGS4) were deleted. Additionally, all waters were deleted. Polar hydrogens were added and optimized using the Kollman United Atom force field. This was followed by addition of all the remaining hydrogens. MOLCAD was then used to generate a surface for all residues thought to be involved in binding of T182 (Gα-binding site). These RGS4-core residues include, I21, I27, F30, F33, L34, E37, S39, N42, I43, W46, I110, L113, M114, D117, S118, R121. The surface area was calculated based on the MOLCAD surface. MOLCAD was also used to calculate the surface area for the identical residues of the free RGS4 NMR structure. The surface area for the free RGS4 NMR structure was calculated to be 404.56 Å². The surface area for the crystal structure was calculated to be 321.88 Å². The difference in surface area of 82.67 Å², is an approximate 20% change in surface area between the two structures. A MOLCAD surface generated on the methyl and hydroxyl groups of T182 of Gα has a surface area of 57.72 Å².

### Example 3

### Identification of an Allosteric Binding Site in RGS4-Core Bead Precipitation Assay for Inhibition of RGS Binding to Gα

Radiolabled [³⁵S]-Gαi1 was synthesized in a rabbit reticulocyte lysate in vitro translation reaaction (Promega, Madison, Wis. Cat. NO. 14960) programmed with in vitro transcribed cRNA preparations (Promega, Cat. No. P1290). Affinity-purified GST-RGS4 core (100 μg, about 25 nM final concentration) is incubated with 17.5 μL glutathione-Sepharose 4B bead (Amersham Pharmacia, Piscataway, N.J., Cat. NO. 17-0756-01) slurry in 100 μL binding buffer (1×PBS, 1 mM MgCl₂, 1 mM DTT, 1% BSA) in a 96-well microtube assay plate. Approximately 300 μM of test compound (about 0.1 mg/mL final concentration, either as a mixture or individual compound) is added to each well and incubated at 4° C. for 30 min. Approximately 50,000-100,000 cpm (?)³⁵S]-Gαi1 in 100 μL assay buffer (1×PBS, 1 mM MgCl₂,-10 μM GDP, 1 mM DTT, 30 μM AlCl₃, 1% BSA, 500 μM NaF) is added to the reaction and incubated at 4° C. for 30 min. The resulting assay sample has a final concentration of about 1-3 nM activated Gαi1. Reaction plates were centrifuged at 1000×g for 3 min, and the supernatant aspirated. Beads were washed 2×by resuspension in 200 μL binding buffer followed by centrifugation. Bound [³⁵S]-Gαi1 is eluted from the bead pellets by resuspending them in 100 μL 1% SDS. Elutates are either counted in 4 mL scintillation fluid or subjected to gel electrophoresis. Random small molecules can be evaluated in the assay described using a compressed library wherein a plurality of test compounds are combined in a single well (e.g., 10 compounds/well for 3000 primary assays tests 30,000 test compounds). Mixtures of test compounds that exhibited a greater than 50% decrease in precipitated radioactivity were confirmed by re-screening in an identical format. Combi-wells (here 10 test compounds/well) that tested positive in both assays were deconvoluted and the individual compounds were tested individually in an identical bead precipitation assay. Compounds that demonstrated the requisite decrease (about 50% or more) in precipitated radioactivity were further tested to confirm that the decrease in precipitated radioactivity was dependent on the RGS4-Gα interaction and not due to spurious activity of the test compound. In these cases, the assay precipitate was analyzed by gel electrophoresis to confirm the presence of RGS4 in the precipitate.

¹H-¹⁵N HSQC Chemical Shift Perturbation

The RGS4 NMR samples contained 0.3 mM of RGS4-core protein in a sample buffer (50 mM KPO₄, 2 mM NaN₃, and 50 mM deuterated DTT in 90% H₂O/10% D₂O at pH 6.0). Test compounds were added to the sample in 10-fold molar excess. 2D 1H-15N HSQC spectra for free RGS4 and RGS4 in the presence of test compounds were collected over a pH titration range of 5.5-6.5. The spectral width in the indirectly detected ¹⁵N dimension was 30.00 ppm with the carrier position at 119.1 ppm. Spectral width in the acquisition dimension was 13.44 ppm with the carrier at the water frequency (4.73 ppm). The number of points acquired in the two dimensions was 256 complex in F1(¹⁵N) and 1024 real in F2(¹H). All spectra were recorded at 35° C. on a Brucker AMX-2 600 spectrometer using a gradient enhanced triple resonance ¹H/¹³C/¹⁵N probe. Water suppression was achieved in the indirectly detected dimension with the WATERGATE sequence and water-flip back pulses (23, 24). Quadrature detection in the indirectly detected dimensions were recorded with States-TPPI hypercomplex phase increment (25). Spectra were collected with appropriate refocusing delays to allow for 0,0 phase correction, processed using the NMRPipe software package (36)) and analyzed with PIPP (37) on a Sun Ultra 10 Workstation. Data processing included a solvent filter, a skewed sine-bell apodization function and one zero-filing in all dimensions.

GTPase Functional Assay A single-turnover GTP-ase assay of G-protein α subunits was used. In this assay GTPase-induced hydrolysis of [γ-³²P]-GTP results in precipitation of radiolabel as ³²Pi. Unhydrolyzed [γ-³²P]-GTP is separated from precipitated label which is then counted. Precipitated label ³²Pi is directly proportional to the amount of [γ-³²P]-GTP hydrolyzed and to the activity of the Gα GTPase .

Purified [γ-³²P]-GTP bound Gα is prepared by incubating Gα (2 μM) with [γ-³²P]-GTP (2 μM) in a reaction buffer (total volume 30 μL), 10 mM Hepes (pH 8.0), 5 mM EDTA, 2 mM DTT, 0.05% C12E10 (Lubrol, ICN Biomedicals, Inc., Aurora, Ohio), 10 μg/mL BSA) for 30 min at 30° C. Unbound [γ-³²P]GTP is removed using a gel filtration column (Centri-Sep, Princeton Separations, Princeton, N.J.) according to the manufacturers directions. The eluate containing [γ-³²P]GTP bound Gα is collected and the protein is recovered (typically up to about 80-90%) after centrifugation at 2000 rpm for 2 min at 4° C.

All steps of the assay are performed at 4° C. The purified [γ-³²P]GTP bound Gα obtained above is added to 500 μL of reaction buffer (as above) and separated in to eight 50 μL samples (a zero time control (no initiation) and seven assay time points). The reaction is initiated by adding 10 μL of 1M MgCl₂ and 10 μL of 10 mM GTP to the seven assay samples. After 10, 20, 30, 40, 60, 90, and 120 seconds, respectively, 750 μL of stop buffer (50 mM NaPO₄ (pH 3.0), 5% activated charcoal) is added to one of the assay samples. The control and samples are then centrifuged at 100,000 rpm for 10 min to precipitate the charcoal and 500 μL of supernatant is remove to assay radiolabel present. GTPase activity is expressed as the amount of free [³²P]-phosphate released from [γ-³²P]GTP. Phosphate release (fmol)=radioactivity (zero time control−time assay)(counts)/specific activity of [γ-³²P]GTP.

GTPase activity of Gαi in the presence of RGS4-GST fusion protein was determined as described above where GTP hydrolysis by 100 nM Gαi was initiated by the addition of MgCl₂ in the presence and absence of 100 nM RGS4-GST protein. GTP hydrolysis at the indicated time points was calculated as the amount of ³²Pi released (in fmol). The dose-dependent effect of RGS4-GST protein on the hydrolysis of GTP-Gαi was measured as described above in the presence or absence of 10 nM or 100 nM RGS4-GST protein.

The effects of test compounds are evaluated for modification of the activity of the RGS4 core domain. The RGS4 core protein was generated as a GST-RGS4core fusion using standard molecular techniques. Briefly, the core region of RGS4 was obtained using PCR to generate a cDNA fragment encoding amino acid 51 (val) to the C-terminal end of the protein, amino acid 206 (ala). The 5′ forward amplification primer contained an embedded BamHI restriction site, followed by nucleotides encoding a flexible linker, Gly-Ser-Gly-Ser, prior to the Val residue of rat RGS4. The 3′ reverse amplification primer contained a stop codon, followed by an embedded BamHI site. The amplimers were used with pWE2RGS4 (Shuey et al., 1998 (84) ) as template to generate a PCR product of approximately 625 base pairs. This PCR product was BamHI digested, purified and ligated in the BamHI site of pGEX-2T (Ammersham Pharmacia, Piscataway N.J.) to generate pGST-RGS4c recombinant plasmid. Plasmid was tranfected into bacterial cells, and DNA prepared by standard methods, and confirmed by sequence analysis. GST-RGS4c fusion protein was generated and purified according to manufacturer suggestions for expression using the pGEX-2T vector.

To measure the effect of test compounds on the activity of RGS4 core domain, RGS4-GST fusion protein (1.6 μM) is incubated with test compound (or mixtures of test compounds) (30 μM-40 μM each) or DMSO for 1 hr at 30° C. Thereafter, GTPase activity of Gαi (100 nM) is measured in the presence or absence of the RGS4-GST treated with the test compound (100 nM). Each assay is replicated at least three-times. RGS4-GST was treated with Compound 1 at 30 μM and inhibited the GTPase activity of RGS by 30%; while RGS4-GST treated with Compound 1 at 300 μM inhibited GTPase activity in comparison to a DMSO control.

Those of ordinary skill in the art will appreciate that reagents, methods, procedures and techniques other than those specifically disclosed herein are known in the art and can be readily employed or adapted to the practice of this invention to achieve the results of this invention. All such art-known functionally equivalent reagents, methods, procedures and techniques are intended to be encompassed by this invention. All references cited herein are incorporated by reference herein in their entirety to the extent that they are not inconsistent with the disclosure herein.

TABLE 1
¹⁵N, ¹³C, ¹³CO and ¹H resonance assignments for RGS4 at pH 6.0 and 30° C.^{a}

| Residue | N | CO | Cα | Cβ | Others |
|---|---|---|---|---|---|
| M1 | —(—) | 176.1 | 55.9 (4.30) | 29.4 (2.10, 2.00) | Cγ33.9 (2.38); Cε21.7 (0.36) |
| R2 | 123.0 (8.34) | 177.2 | 56.7 (4.29) | 33.0 (1.84, 1.77) | Cγ24.8 (1.45); Cδ29.2 (1.71); Cε42.2 (3.00) |
| G3 | 110.4 (8.40) | 173.7 | 45.3 (4.00) | | |
| S4 | 115.7 (8.23) | 174.0 | 58.1 (4.59) | 64.2 (3.90) | |
| V5 | 121.6 (8.20) | 174.9 | 61.2 (4.38) | 33.6 (1.99) | Cγ21.7 (0.98); 21.9 (0.89) |
| S6 | 122.3 (8.56) | 175.1 | 57.5 (4.51) | 65.3 (4.33, 4.02) | |
| Q7 | 121.5 (8.95) | 178.2 | 58.5 (3.80) | 28.3 (2.02) | Cγ34.3 (2.39) |
| E8 | 118.9 (8.48) | 178.8 | 59.7 (3.85) | 29.1 (2.01, 1.94) | Cγ36.6 (2.29) |
| E9 | 120.5 (7.43) | 177.0 | 58.9 (3.77) | 29.4 (2.04, 1.72) | Cγ36.4 (2.23) |
| V10 | 116.8 (7.16) | 180.8 | 65.0 (3.89) | 31.4 (1.54) | Cγ22.1 (0.34); 22.6 (0.34) |
| K11 | 122.0 (7.86) | 179.6 | 59.9 (3.92) | 32.1 (1.82) | Cγ25.4 (1.52, 1.37); Cδ29.4 (1.62); |
| | | | | | Cε42.1 (2.91, 2.79) |
| K12 | 120.0 (7.39) | 180.4 | 59.1 (4.09) | 31.7 (2.12, 1.93) | Cγ25.4 (1.52, 1.45); Cδ29.2 (1.68); |
| | | | | | Cε42.0 (2.99) |
| W13 | 120.7 (7.84) | 176.7 | 57.2 (4.63) | 29.8 (3.46) | Cδ1 126.4 (6.67); Nε1 129.3 (9.69); |
| | | | | | Cζ2 113.9 (6.25); Cη2 124.0 (6.81); |
| | | | | | Cζ3 121.3 (7.18) |
| A14 | 117.5 (7.48) | 176.9 | 52.3 (4.36) | 18.6 (1.52) | |
| E15 | 117.5 (7.80) | 177.1 | 57.6 (4.29) | 30.9 (2.16) | Cγ36.6 (2.48, 2.25) |
| S16 | 111.6 (7.02) | 173.6 | 57.5 (4.50) | 65.0 (4.22, 3.93) | |
| L17 | 125.4 (8.36) | 177.5 | 57.1 (3.17) | 39.5 (0.97) | Cγ26.2 (0.65); Cδ25.9 (−0.48); 23.9 (−0.08) |
| E18 | 117.2 (8.68) | 177.7 | 60.1 (3.63) | 29.3 (1.98, 1.91) | Cγ36.5 (2.19) |
| N19 | 116.0 (7.53) | 176.9 | 55.3 (4.37) | 38.4 (3.20, 2.78) | |
| L20 | 119.5 (7.01) | 176.4 | 58.2 (3.50) | 41.8 (1.79, 1.63) | Cγ24.7 (0.52); Cδ26.9 (−0.03); 24.9 (0.33) |
| I21 | 105.6 (7.37) | 175.1 | 63.3 (3.65) | 37.1 (1.61) | Cγm 19.2 (0.33); Cγ(0.70, 0.81); |
| | | | | | Cδ12.3 (−0.62) |
| N22 | 115.7 (7.10) | 173.8 | 53.9 (4.76) | 39.5 (2.88) | |
| H23 | 121.6 (7.32) | 176.0 | 56.4 (4.71) | 33.9 (3.31, 2.98) | Cδ2 138.6 (8.15) |
| E24 | 128.2 (9.04) | 179.5 | 60.6 (4.07) | 29.8 (2.09) | Cγ36.1 (2.44, 2.34) |
| C25 | 121.4 (10.57) | 176.9 | 61.8 (4.46) | 27.3 (3.37) | |
| G26 | 114.1 (7.36) | — | 46.6 (2.86, 2.45) | | |
| L27 | 122.8 (8.66) | 178.3 | 58.2 (3.76) | 41.4 (1.72, 1.65) | Cγ27.1 (1.60); Cδ25.6 (1.05); 23.5 (1.04) |
| A28 | 118.3 (7.29) | 180.8 | 55.3 (4.06) | 18.4 (1.60) | |
| A29 | 122.8 (7.85) | 178.9 | 55.3 (4.51) | 18.2 (1.84) | |
| F30 | 120.0 (8.51) | 177.8 | 59.1 (3.94) | 39.5 (2.62, 2.38) | Cδ- (6.34) |
| K31 | 118.9 (9.02) | 177.5 | 60.6 (3.59) | 32.4 (1.94, 1.74) | Cγ26.0 (1.70, 1.44); Cδ- (1.63); Cε- (2.96) |
| A32 | 121.6 (7.82) | 180.3 | 55.3 (3.98) | 17.8 (1.61) | |
| F33 | 121.5 (7.82) | 177.8 | 59.9 (4.39) | 39.2 (3.18, 2.00) | Cδ- (6.78); Cε- (7.18) |
| L34 | 122.0 (8.57) | 180.0 | 57.6 (3.34) | 40.2 (0.88) | Cγ26.2 (1.01); Cδ21.8 (0.57); 26.3 (0.19) |
| K35 | 120.9 (8.60) | 180.1 | 59.6 (3.90) | 32.1 (1.79) | Cγ24.9 (1.54, 1.39); Cε- (2.93) |
| S36 | 116.7 (7.43) | 174.1 | 61.1 (4.09) | 63.1 (4.04, 2.38) | |
| E37 | 118.0 (6.86) | 175.3 | 55.3 (4.28) | 29.9 (1.98, 1.45) | Cγ35.9 (1.66) |
| Y38 | 118.8 (7.60) | 175.8 | 58.7 (4.37) | 35.5 (3.27, 3.09) | Cδ133.4 (7.04); Cε118.2 (6.84) |
| S39 | 113.0 (8.05) | 175.5 | 57.0 (5.04) | 64.5 (3.83, 3.71) | |
| E40 | 122.2 (9.07) | 175.9 | 58.2 (3.86) | 30.2 (1.96, 1.75) | Cγ36.9 (2.20) |
| E41 | 122.3 (10.22) | 177.2 | 60.9 (4.25) | 27.7 (2.13, 1.98) | Cγ35.6 (2.37) |
| N42 | 116.4 (7.67) | 176.9 | 56.7 (4.52) | 38.8 (2.68) | |
| I43 | 117.6 (6.99) | 176.2 | 58.9 (4.52) | 38.3 (1.58) | Cγm 20.7 (1.05); Cδ14.6 (0.92) |
| D44 | 126.2 (8.38) | 179.6 | 57.6 (4.59) | 39.1 (2.85) | |
| F45 | 123.8 (8.40) | 176.5 | 60.2 (4.37) | 39.0 (2.98, 2.50) | Cδ- (6.67) |
| W46 | 122.3 (7.98) | 177.7 | 64.9 (4.39) | 29.9 (3.78, 3.21) | Nε1 130.7 (10.68); Cζ2 115.1 (7.25); |
| | | | | | Cη2 125.8 (7.01); Cζ3 122.9 (7.01); |
| | | | | | Cε3 119.9 (7.35) |
| I47 | 118.2 (9.04) | 178.4 | 65.1 (3.37) | 38.6 (2.02) | Cγm 17.0 (0.95); Cγ29.4 (2.24, 1.46); |
| | | | | | Cδ14.3 (1.07) |
| S48 | 117.4 (7.91) | 176.6 | 62.8 (4.22) | —(3.89, 3.67) | |
| C49 | 120.4 (7.53) | 179.6 | 64.0 (3.90) | —(3.61) | |
| E50 | 120.1 (7.39) | 179.1 | 58.8 (3.58) | 29.0 (1.65) | Cγ35.2 (—) |
| E51 | 118.3 (7.93) | 180.0 | 59.0 (3.84) | 29.7 (2.21, 1.95) | Cγ36.3 (2.15) |
| Y52 | 123.0 (8.11) | 175.6 | 61.2 (3.95) | 38.7 (3.30, 2.95) | Cδ- (7.03); Cε118.5 (7.15) |
| K53 | 112.6 (7.43) | 177.1 | 58.2 (3.89) | 32.2 (1.86, 1.76) | Cγ- (1.54, 1.39) |
| K54 | 117.5 (7.18) | 176.9 | 56.1 (4.17) | 33.1 (1.98, 1.71) | Cγ25.4 (—); Cδ29.4 (1.60); Cε42.2 (—) |
| I55 | 123.1 (7.39) | 176.8 | 64.0 (3.58) | 37.2 (1.74) | Cγm 19.4 (0.75); Cγ23.3 (0.35) |
| K56 | 126.5 (8.39) | 176.7 | 56.3 (4.34) | 33.6 (1.95, 1.72) | Cγ24.6 (1.40); Cδ28.7 (1.64); Cε42.1 (3.00) |
| S57 | 116.2 (7.35) | — | 54.6 (4.98) | 64.0 (3.79) | |
| P58 | —(—) | 178.8 | 65.0 (4.19) | 32.1 (2.45, 2.06) | Cγ27.5 (2.17, 2.06); Cδ51.4 (4.11, 3.98) |
| S59 | 112.3 (8.15) | 175.5 | 60.5 (4.32) | 62.7 (3.90) | |
| K60 | 119.7 (7.77) | 177.6 | 56.3 (4.36) | 33.2 (2.03, 1.89) | Cγ25.7 (—); Cδ29.0 (—); Cε42.4 (—) |
| L61 | 120.4 (7.49) | 178.5 | 58.7 (4.12) | 41.3 (1.82, 1.71) | Cγ26.8 (1.67); Cδ25.9 (0.74); 23.2 (0.35) |
| S62 | 112.7 (8.67) | — | 64.0 (4.38) | 60.8 (4.08) | |
| P63 | —(—) | 180.0 | 66.0 (4.39) | 30.8 (2.42, 1.95) | |
| K64 | 118.0 (7.08) | 178.0 | 57.9 (4.36) | 31.9 (2.26) | |
| A65 | 122.5 (9.23) | 179.9 | 55.5 (4.19) | 17.7 (1.76) | |
| K66 | 116.9 (8.40) | 178.4 | 60.2 (3.99) | 32.4 (1.93) | Cγ25.8 (1.43); Cδ29.2 (1.68); Cε42.1 (3.00) |
| K67 | 119.5 (7.52) | 179.6 | 59.9 (4.14) | 32.7 (2.09) | Cγ25.2 (1.66, 1.52) |
| I68 | 120.9 (8.24) | 178.4 | 65.9 (3.87) | 38.3 (2.08) | Cγm 18.3 (0.93); Cδ- 0.30 |
| Y69 | 120.8 (9.15) | 178.6 | 62.4 (3.99) | 39.5 (3.52, 3.26) | Cδ133.4 (7.07); Cε117.9 (6.78) |
| N70 | 117.5 (8.77) | 176.0 | 55.7 (4.42) | 38.4 (2.99, 2.78) | Nγ112.0 (7.59, 6.97) |
| E71 | 118.4 (7.88) | 176.4 | 58.8 (4.00) | 30.6 (1.76, 1.37) | Cγ35.9 (1.80, 1.09) |
| F72 | 110.4 (7.76) | 175.1 | 58.5 (4.95) | 43.4 (2.77) | Cδ133.6 (7.75); Cε133.1 (7.19); Cζ - (6.89) |
| I73 | 116.5 (7.59) | 175.3 | 61.3 (4.05) | 38.0 (1.59) | Cγ27.2 (1.43, 1.03); Cγm 18.5 (0.79); |
| | | | | | Cδ12.0 (0.76) |
| S74 | 113.4 (7.43) | 174.6 | 57.7 (3.89) | 64.1 (3.44) | |
| V75 | 120.6 (8.20) | 177.1 | 64.4 (3.99) | 31.3 (2.18) | Cγ20.3 (1.02); 21.1 (1.02) |
| Q76 | 118.6 (8.10) | 175.4 | 55.7 (4.29) | 28.2 (2.23, 1.87) | Cγ34.6 (2.30) |
| A77 | 124.1 (7.54) | 178.7 | 52.8 (4.11) | 18.8 (1.10) | |
| T78 | 114.8 (7.99) | 175.6 | 64.0 (4.05) | 68.8 (4.11) | Cγ22.6 (1.27) |
| K79 | 123.8 (7.89) | 173.4 | 54.6 (4.47) | 33.1 (1.76, 1.39) | Cγ25.2 (1.27); Cδ29.7 (1.64, 1.51); |
| | | | | | Cε42.0 (2.84) |
| E80 | 117.8 (7.16) | 176.2 | 56.6 (3.97) | 30.0 (1.82) | Cγ34.5 (2.69, 2.19) |
| V81 | 119.3 (8.22) | 176.3 | 59.5 (4.56) | 33.4 (2.01) | Cγ22.5 (0.89); 17.5 (0.34) |
| N82 | 122.5 (9.09) | 173.7 | 53.2 (4.64) | 37.5 (2.89, 2.63) | |
| L83 | 122.7 (7.43) | 176.1 | 53.1 (4.52) | 46.3 (1.49, 1.34) | Cγ26.4 (—); Cδ24.3 (0.94) - (0.73) |
| D84 | 120.3 (8.27) | 176.3 | 53.2 (4.66) | 41.7 (3.03, 2.79) | |
| S85 | 116.6 (8.93) | 176.7 | 62.5 (4.00) | —(4.01) | |
| C86 | 121.4 (8.36) | 177.9 | 62.4 (4.30) | 26.1 (3.07) | |
| T87 | 120.2 (8.35) | 178.5 | 67.0 (4.06) | 67.0 (4.06) | Cγ22.7 (1.22) |
| R88 | 125.5 (8.55) | 178.2 | 61.6 (3.73) | 29.9 (1.98, 1.91) | Cγ- (1.51, 1.67); Cδ43.3 (3.10) |
| E89 | 121.1 (8.49) | 179.5 | 59.6 (4.12) | 29.2 (2.17, 2.08) | Cγ36.2 (2.39) |
| E90 | 121.2 (8.44) | 178.5 | 59.5 (4.01) | 29.4 (2.17) | Cγ35.6 (2.40) |
| T91 | 115.1 (8.10) | 175.8 | 67.8 (3.91) | 68.4 (4.42) | Cγ22.3 (1.43) |
| S92 | 116.6 (8.32) | 176.6 | 62.4 (3.90) | 62.7 (4.09, 3.99) | |
| R93 | 122.3 (7.84) | 179.6 | 59.3 (4.05) | 29.9 (1.95, 1.91) | Cγ27.9 (1.82, 1.60); Cδ43.5 (3.23) |
| N94 | 121.6 (8.10) | 176.7 | 54.9 (4.33) | 38.6 (3.18) | |
| M95 | 115.7 (7.40) | 176.9 | 55.1 (4.07) | 31.3 (1.83, 1.77) | Cγ31.7 (2.29, 1.94); Cε15.3 (1.67) |
| L96 | 119.8 (7.29) | 178.4 | 57.7 (4.02) | 41.5 (1.80, 1.55) | Cγ27.0 (1.80); Cδ23.0 (0.85); 24.8 (0.95) |
| E97 | 116.6 (7.20) | — | 53.8 (4.52) | 30.1 (1.92, 1.85) | Cγ36.1 (2.10) |
| P98 | —(—) | 176.9 | 64.4 (4.34) | 32.1 (2.26, 1.93) | Cγ27.9 (2.08); Cδ49.9 (3.59) |
| T99 | 113.7 (8.43) | 179.1 | 59.6 (4.79) | 72.5 (4.60) | Cγ21.1 (1.30) |
| I100 | 123.2 (9.19) | 176.1 | 63.2 (4.22) | 39.6 (1.97) | Cγm 19.1 (1.04); Cγ28.5 (1.47, 1.39); |
| | | | | | Cδ14.8 (0.90) |
| T101 | 109.8 (7.61) | 176.2 | 61.5 (4.57) | 69.3 (4.63) | Cγ21.7 (1.20) |
| C102 | 122.7 (7.48) | 174.5 | 62.6 (3.85) | 28.9 (3.15, 2.31) | |
| F103 | 112.8 (8.78) | 175.2 | 57.8 (4.95) | 40.7 (3.71, 2.57) | Cδ- (6.76) |
| D104 | 122.0 (7.72) | 179.1 | 58.9 (4.28) | 39.8 (2.82, 2.59) | |
| E105 | 119.9 (8.75) | 175.0 | 59.4 (4.16) | 28.6 (2.44, 2.12) | Cγ35.9 (2.42) |
| A106 | 122.9 (8.66) | 178.8 | 55.4 (4.03) | 18.6 (1.65) | |
| Q107 | 118.7 (9.58) | 178.3 | 60.3 (3.90) | 27.9 (—) | Cγ35.2 (—) |
| K108 | 120.1 (8.11) | 179.3 | 59.6 (4.21) | 32.4 (2.18, 2.02) | Cγ25.2 (1.46); Cδ29.8 (1.69); Cε42.2 (3.00) |
| K109 | 118.8 (8.08) | 180.4 | 59.0 (4.27) | 31.8 (2.22, 2.04) | Cγ24.9 (1.44); Cδ29.1 (1.71, 1.81); |
| | | | | | Cε42.0 (3.00) |
| I110 | 121.3 (8.30) | 177.9 | 61.5 (4.22) | 36.0 (2.44) | Cγ29.0 (2.06, 1.43); Cγm 19.6 (1.38); |
| | | | | | Cδ10.0 (0.76) |
| F111 | 124.5 (9.22) | 176.8 | 63.2 (3.80) | 39.2 (3.51, 3.21) | Cδ131.9 (6.87); Cε- (7.04) |
| N112 | 117.2 (8.51) | 177.5 | 56.3 (4.50) | 38.6 (3.08, 2.90) | |
| L113 | 121.5 (7.95) | 180.3 | 58.3 (4.21) | 42.3 (1.99) | Cγ27.1 (1.83); Cδ24.9 (1.06) |
| M114 | 11.8.6 (8.22) | 177.0 | 60.0 (4.28) | 35.0 (1.82, 1.68) | Cγ32.0 (2.38, 2.26); Cε15.9 (1.73) |
| E115 | 120.2 (8.46) | 177.3 | 60.4 (3.49) | 29.6 (1.58) | Cγ37.3 (2.07) |
| K116 | 113.5 (7.57) | 176.9 | 57.5 (4.26) | 33.1 (1.98) | Cγ24.7 (1.59); Cδ29.5 (1.72); Cε42.3 (3.04) |
| D117 | 116.6 (7.62) | 176.6 | 55.8 (5.08) | 42.8 (2.92, 2.84) | |
| S118 | 117.2 (8.45) | 176.2 | 63.0 (4.52) | 63.6 (4.33, 4.02) | |
| Y119 | 121.8 (8.95) | 175.5 | 60.4 (4.27) | 39.2 (2.81, 2.00) | Cδ- (6.72); Cε117.2 (6.58) |
| R120 | 114.4 (7.11) | 179.8 | 58.3 (3.52) | 28.7 (1.96, 1.80) | Cγ27.0 (1.87, 1.64); Cδ42.9 (3.16, 3.10) |
| R121 | 117.5 (7.63) | 179.4 | 59.6 (3.97) | 30.3 (2.07) | Cδ43.1 (—) |
| F122 | 124.0 (8.57) | 178.4 | 60.0 (3.32) | 38.3 (2.89, 2.76) | Cδ- (6.77) |
| L123 | 117.7 (7.05) | 176.3 | 56.3 (3.27) | 41.4 (1.11, 1.06) | Cγ25.6 (1.31); Cδ25.2 (0.32); 19.6 (−0.09) |
| K124 | 114.3 (6.72) | 175.5 | 54.8 (4.42) | 33.0 (2.09, 1.63) | Cγ24.8 (1.46); Cδ28.9 (1.70); Cε42.2 (2.97) |
| S125 | 118.2 (7.83) | 175.6 | 57.9 (4.79) | 67.1 (4.52) | |
| R126 | 121.1 (9.39) | 176.5 | 58.2 (3.92) | 28.4 (1.81) | Cγ25.5 (1.60, 1.43); Cδ42.5 (3.05, 2.97) |
| F127 | 115.5 (7.47) | 176.0 | 60.7 (4.21) | 39.5 (3.42, 3.27) | Cδ- (7.18) |
| Y128 | 114.2 (6.68) | 177.2 | 59.3 (4.40) | 39.8 (2.39) | Cδ133.0 (7.17); Cε117.6 (6.91) |
| L129 | 116.8 (8.34) | 180.4 | 57.6 (3.71) | 41.7 (1.41, 1.37) | Cγ26.8 (1.76); Cδ25.6 (0.74); 22.4 (0.89) |
| D130 | 119.0 (8.61) | 177.8 | 56.8 (4.38) | 39.8 (2.63) | |
| L131 | 117.5 (7.46) | 177.8 | 55.7 (4.18) | 42.1 (1.83, 1.43) | Cγ22.9 (1.27); Cδ22.4 (0.94); 22.6 (0.33) |
| T132 | 108.3 (7.24) | 173.9 | 61.6 (4.26) | 70.1 (4.09) | Cγ21.6 (0.73) |
| N133 | 119.8 (7.60) | 173.0 | 51.3 (4.95) | 38.8 (2.78, 2.62) | |
| P134 | —(—) | 177.4 | 63.7 (4.43) | 32.1 (2.27, 1.96) | Cγ27.2 (1.97); Cδ50.6 (3.67, 3.50) |
| S135 | 115.5 (8.32) | 174.9 | 58.8 (4.44) | 63.7 (4.42, 3.88) | |
| S136 | 117.6 (8.22) | 174.7 | 58.5 (4.47) | 63.8 (3.90, 3.13) | |
| C137 | 120.7 (8.27) | 175.1 | 58.7 (4.55) | 28.1 (2.95) | |
| G138 | 111.5 (8.43) | 173.8 | 45.4 (3.96) | —(—) | |
| A139 | 124.0 (8.10) | 178.0 | 52.6 (4.30) | 19.4 (1.43) | |
| E140 | 120.0 (8.48) | 176.8 | 57.0 (4.22) | 30.0 (2.01) | Cγ36.2 (2.28) |
| K141 | 122.1 (8.23) | 176.7 | 56.5 (4.27) | 32.9 (1.80) | Cγ24.9 (1.44); Cδ29.0 (1.71); Cε42.2 (3.00) |
| Q142 | 121.1 (8.27) | 173.1 | 55.0 (4.16) | 33.1 (2.21) | Cγ30.9 (2.64) |
| K143 | 120.7 (8.27) | 176.3 | 56.7 (4.42) | 30.8 (1.92, 1.86) | Cγ24.7 (—); Cδ27.1 (1.71); Cε43.5 (3.25) |
| G144 | 111.1 (8.57) | 176.1 | 45.4 (3.97) | —(—) | |
| A145 | 124.0 (8.15) | 177.9 | 52.7 (4.30) | 19.5 (1.43) | |
| K146 | 120.9 (8.35) | 179.9 | 56.3 (4.35) | 33.1 (1.81, 1.45) | Cγ24.8 (1.46); Cδ29.1 (1.71, 1.84); |
| | | | | | Cε42.2 (3.01) |
| S147 | 117.3 (8.39) | 174.8 | 58.2 (3.94) | 64.1 (4.50, 3.90) | |
| S148 | 118.3 (8.39) | 174.5 | 58.5 (4.47) | 63.9 (3.90) | |
| A149 | 125.7 (8.30) | 177.5 | 52.8 (4.29) | 19.3 (1.38) | |
| D150 | 119.3 (8.18) | 176.5 | 54.4 (4.61) | 41.2 (2.70) | |
| C151 | 119.8 (8.28) | 175.1 | 58.8 (4.58) | 27.9 (2.98) | |
| T152 | 116.4 (8.28) | 174.7 | 62.5 (4.33) | 69.7 (4.27) | Cγ21.7 (1.24) |
| S153 | 118.0 (8.20) | 174.2 | 58.4 (4.47) | 63.8 (3.93, 3.87) | |
| L154 | 124.3 (8.18) | 177.0 | 55.2 (4.37) | 42.4 (1.61) | Cγ27.0 (1.61); Cδ23.5 (0.86); 24.9 (0.90) |
| V155 | 122.4 (8.01) | 174.4 | 59.9 (4.39) | 32.6 (2.06) | Cγ21.0 (0.94); 20.4 (0.94) |
| P156 | —(—) | 177.0 | 63.3 (4.39) | 32.2 (2.30, 1.90) | Cγ27.5 (2.03, 1.94); Cδ51.1 (3.86, 3.69) |
| Q157 | 120.9 (8.49) | 176.1 | 56.1 (4.29) | 29.5 (2.06, 2.00) | Cγ33.9 (2.41) |
| C158 | 120.2 (8.31) | 174.1 | 58.2 (4.45) | 28.2 (2.88) | |
| A159 | 126.3 (8.32) | 177.3 | 52.6 (4.25) | 19.3 (1.30) | |
| H160 | 117.9 (8.24) | — | 55.5 (4.62) | 29.8 (3.15, 3.06) | Cδ2 119.5 (7.13) |

Footnotes to Table S1
^{a}In each column, ¹⁵N and ¹³C shifts are listed first, and the corresponding ¹H shifts are given in parentheses. ¹H and ¹³C chemical shifts are reported relative to 3-(trimethylsily)propionic-d₄ acid and ¹⁵N shifts relative to external liguid NH₃.

TABLE 2
Restrained Minimized NMR Coordinates for Free RGS4
Pages 42-70
The structural coordinates herein are deposited with Brookhaven
Protein Database.(Brookhaven National Laboratory)
Deposit No.

| | | | | | | | | | |
|---|---|---|---|---|---|---|---|---|---|
| ATOM | 1 | N | VAL | 5 | −8.546 | 2.447 | −13.971 | 1.00 | 0.99 |
| ATOM | 2 | HN | VAL | 5 | −8.477 | 3.418 | −14.081 | 1.00 | 1.07 |
| ATOM | 3 | CA | VAL | 5 | −9.109 | 1.878 | −12.714 | 1.00 | 0.86 |
| ATOM | 4 | HA | VAL | 5 | −8.922 | 0.815 | −12.685 | 1.00 | 0.89 |
| ATOM | 5 | CB | VAL | 5 | −8.442 | 2.546 | −11.509 | 1.00 | 0.82 |
| ATOM | 6 | HB | VAL | 5 | −8.686 | 3.599 | −11.500 | 1.00 | 1.23 |
| ATOM | 7 | CG1 | VAL | 5 | −8.941 | 1.895 | −10.217 | 1.00 | 1.02 |
| ATOM | 8 | HG11 | VAL | 5 | −10.011 | 2.014 | −10.143 | 1.00 | 1.62 |
| ATOM | 9 | HG12 | VAL | 5 | −8.467 | 2.368 | −9.370 | 1.00 | 1.48 |
| ATOM | 10 | HG13 | VAL | 5 | −8.695 | 0.844 | −10.226 | 1.00 | 1.53 |
| ATOM | 11 | CG2 | VAL | 5 | −6.925 | 2.373 | −11.609 | 1.00 | 1.37 |
| ATOM | 12 | HG21 | VAL | 5 | −6.540 | 2.024 | −10.663 | 1.00 | 1.93 |
| ATOM | 13 | HG22 | VAL | 5 | −6.470 | 3.321 | −11.855 | 1.00 | 1.76 |
| ATOM | 14 | HG23 | VAL | 5 | −6.696 | 1.652 | −12.380 | 1.00 | 1.88 |
| ATOM | 15 | C | VAL | 5 | −10.617 | 2.132 | −12.671 | 1.00 | 0.82 |
| ATOM | 16 | O | VAL | 5 | −11.079 | 3.227 | −12.927 | 1.00 | 0.84 |
| ATOM | 17 | N | SER | 6 | −11.389 | 1.130 | −12.350 | 1.00 | 0.82 |
| ATOM | 18 | HN | SER | 6 | −10.997 | 0.255 | −12.147 | 1.00 | 0.83 |
| ATOM | 19 | CA | SER | 6 | −12.866 | 1.318 | −12.292 | 1.00 | 0.83 |
| ATOM | 20 | HA | SER | 6 | −13.194 | 1.870 | −13.160 | 1.00 | 0.89 |
| ATOM | 21 | CB | SER | 6 | −13.550 | −0.049 | −12.268 | 1.00 | 0.89 |
| ATOM | 22 | HB1 | SER | 6 | −13.181 | −0.650 | −13.089 | 1.00 | 0.95 |
| ATOM | 23 | HB2 | SER | 6 | −14.615 | 0.077 | −12.370 | 1.00 | 0.93 |
| ATOM | 24 | OG | SER | 6 | −13.271 | −0.692 | −11.031 | 1.00 | 0.87 |
| ATOM | 25 | HG | SER | 6 | −12.543 | −0.228 | −10.613 | 1.00 | 1.20 |
| ATOM | 26 | C | SER | 6 | −13.234 | 2.093 | −11.025 | 1.00 | 0.74 |
| ATOM | 27 | O | SER | 6 | −12.494 | 2.113 | −10.061 | 1.00 | 0.68 |
| ATOM | 28 | N | GLN | 7 | −14.375 | 2.727 | −11.017 | 1.00 | 0.75 |
| ATOM | 29 | HN | GLN | 7 | −14.960 | 2.695 | −11.803 | 1.00 | 0.82 |
| ATOM | 30 | CA | GLN | 7 | −14.792 | 3.496 | −9.810 | 1.00 | 0.70 |
| ATOM | 31 | HA | GLN | 7 | −13.973 | 4.129 | −9.505 | 1.00 | 0.66 |
| ATOM | 32 | CB | GLN | 7 | −15.999 | 4.376 | −10.139 | 1.00 | 0.76 |
| ATOM | 33 | HB1 | GLN | 7 | −16.404 | 4.793 | −9.230 | 1.00 | 0.75 |
| ATOM | 34 | HB2 | GLN | 7 | −16.754 | 3.780 | −10.633 | 1.00 | 0.82 |
| ATOM | 35 | CG | GLN | 7 | −15.551 | 5.508 | −11.067 | 1.00 | 0.83 |
| ATOM | 36 | HG1 | GLN | 7 | −15.175 | 5.090 | −11.988 | 1.00 | 1.37 |
| ATOM | 37 | HG2 | GLN | 7 | −14.768 | 6.077 | −10.585 | 1.00 | 1.26 |
| ATOM | 38 | CD | GLN | 7 | −16.733 | 6.428 | −11.375 | 1.00 | 1.51 |
| ATOM | 39 | OE1 | GLN | 7 | −17.840 | 5.972 | −11.578 | 1.00 | 2.25 |
| ATOM | 40 | NE2 | GLN | 7 | −16.540 | 7.720 | −11.412 | 1.00 | 2.18 |
| ATOM | 41 | HE21 | GLN | 7 | −15.646 | 8.086 | −11.243 | 1.00 | 2.33 |
| ATOM | 42 | HE22 | GLN | 7 | −17.288 | 8.322 | −11.606 | 1.00 | 2.90 |
| ATOM | 43 | C | GLN | 7 | −15.130 | 2.533 | −8.666 | 1.00 | 0.66 |
| ATOM | 44 | O | GLN | 7 | −15.090 | 2.888 | −7.507 | 1.00 | 0.65 |
| ATOM | 45 | N | GLU | 8 | −15.470 | 1.317 | −8.979 | 1.00 | 0.69 |
| ATOM | 46 | HN | GLU | 8 | −15.504 | 1.043 | −9.919 | 1.00 | 0.73 |
| ATOM | 47 | CA | GLU | 8 | −15.800 | 0.339 | −7.904 | 1.00 | 0.69 |
| ATOM | 48 | HA | GLU | 8 | −16.529 | 0.773 | −7.237 | 1.00 | 0.71 |
| ATOM | 49 | CB | GLU | 8 | −16.377 | −0.935 | −8.526 | 1.00 | 0.78 |
| ATOM | 50 | HB1 | GLU | 8 | −16.450 | −1.702 | −7.770 | 1.00 | 0.81 |
| ATOM | 51 | HB2 | GLU | 8 | −15.725 | −1.273 | −9.319 | 1.00 | 0.79 |
| ATOM | 52 | CG | GLU | 8 | −17.768 | −0.652 | −9.096 | 1.00 | 0.87 |
| ATOM | 53 | HG1 | GLU | 8 | −17.707 | 0.150 | −9.816 | 1.00 | 1.11 |
| ATOM | 54 | HG2 | GLU | 8 | −18.435 | −0.368 | −8.294 | 1.00 | 1.22 |
| ATOM | 55 | CD | GLU | 8 | −18.300 | −1.912 | −9.781 | 1.00 | 1.57 |
| ATOM | 56 | OE1 | GLU | 8 | −19.406 | −1.864 | −10.292 | 1.00 | 2.30 |
| ATOM | 57 | OE2 | GLU | 8 | −17.590 | −2.905 | −9.784 | 1.00 | 2.12 |
| ATOM | 58 | C | GLU | 8 | −14.535 | −0.020 | −7.113 | 1.00 | 0.62 |
| ATOM | 59 | O | GLU | 8 | −14.569 | −0.196 | −5.911 | 1.00 | 0.61 |
| ATOM | 60 | N | GLU | 9 | −13.429 | −0.178 | −7.793 | 1.00 | 0.61 |
| ATOM | 61 | HN | GLU | 9 | −13.439 | −0.064 | −8.766 | 1.00 | 0.64 |
| ATOM | 62 | CA | GLU | 9 | −12.163 | −0.582 | −7.106 | 1.00 | 0.59 |
| ATOM | 63 | HA | GLU | 9 | −12.353 | −1.452 | −6.496 | 1.00 | 0.63 |
| ATOM | 64 | CB | GLU | 9 | −11.123 | −0.939 | −8.169 | 1.00 | 0.67 |
| ATOM | 65 | HB1 | GLU | 9 | −10.158 | −1.063 | −7.701 | 1.00 | 0.68 |
| ATOM | 66 | HB2 | GLU | 9 | −11.069 | −0.145 | −8.900 | 1.00 | 0.67 |
| ATOM | 67 | CG | GLU | 9 | −11.524 | −2.244 | −8.860 | 1.00 | 0.81 |
| ATOM | 68 | HG1 | GLU | 9 | −12.552 | −2.179 | −9.180 | 1.00 | 1.46 |
| ATOM | 69 | HG2 | GLU | 9 | −11.411 | −3.068 | −8.170 | 1.00 | 1.11 |
| ATOM | 70 | CD | GLU | 9 | −10.628 | −2.471 | −10.080 | 1.00 | 1.55 |
| ATOM | 71 | OE1 | GLU | 9 | −10.815 | −3.474 | −10.749 | 1.00 | 2.28 |
| ATOM | 72 | OE2 | GLU | 9 | −9.772 | −1.636 | −10.325 | 1.00 | 2.22 |
| ATOM | 73 | C | GLU | 9 | −11.603 | 0.542 | −6.221 | 1.00 | 0.52 |
| ATOM | 74 | O | GLU | 9 | −11.118 | 0.290 | −5.135 | 1.00 | 0.51 |
| ATOM | 75 | N | VAL | 10 | −11.644 | 1.770 | −6.659 | 1.00 | 0.50 |
| ATOM | 76 | HN | VAL | 10 | −12.027 | 1.971 | −7.539 | 1.00 | 0.53 |
| ATOM | 77 | CA | VAL | 10 | −11.087 | 2.865 | −5.808 | 1.00 | 0.47 |
| ATOM | 78 | HA | VAL | 10 | −10.061 | 2.627 | −5.566 | 1.00 | 0.50 |
| ATOM | 79 | CB | VAL | 10 | −11.126 | 4.202 | −6.559 | 1.00 | 0.50 |
| ATOM | 80 | HB | VAL | 10 | −10.787 | 4.993 | −5.906 | 1.00 | 0.53 |
| ATOM | 81 | CG1 | VAL | 10 | −10.216 | 4.132 | −7.790 | 1.00 | 0.67 |
| ATOM | 82 | HG11 | VAL | 10 | −9.970 | 5.133 | −8.114 | 1.00 | 1.32 |
| ATOM | 83 | HG12 | VAL | 10 | −10.729 | 3.613 | −8.587 | 1.00 | 1.27 |
| ATOM | 84 | HG13 | VAL | 10 | −9.309 | 3.602 | −7.542 | 1.00 | 1.13 |
| ATOM | 85 | CG2 | VAL | 10 | −12.552 | 4.486 | −7.011 | 1.00 | 0.58 |
| ATOM | 86 | HG21 | VAL | 10 | −13.164 | 4.719 | −6.153 | 1.00 | 1.12 |
| ATOM | 87 | HG22 | VAL | 10 | −12.938 | 3.615 | −7.506 | 1.00 | 1.22 |
| ATOM | 88 | HG23 | VAL | 10 | −12.556 | 5.321 | −7.695 | 1.00 | 1.18 |
| ATOM | 89 | C | VAL | 10 | −11.887 | 2.964 | −4.504 | 1.00 | 0.45 |
| ATOM | 90 | O | VAL | 10 | −11.340 | 3.243 | −3.457 | 1.00 | 0.46 |
| ATOM | 91 | N | LYS | 11 | −13.173 | 2.730 | −4.545 | 1.00 | 0.47 |
| ATOM | 92 | HN | LYS | 11 | −13.607 | 2.498 | −5.393 | 1.00 | 0.48 |
| ATOM | 93 | CA | LYS | 11 | −13.973 | 2.807 | −3.285 | 1.00 | 0.50 |
| ATOM | 94 | HA | LYS | 11 | −13.867 | 3.795 | −2.864 | 1.00 | 0.52 |
| ATOM | 95 | CB | LYS | 11 | −15.456 | 2.528 | −3.561 | 1.00 | 0.56 |
| ATOM | 96 | HB1 | LYS | 11 | −15.979 | 2.413 | −2.623 | 1.00 | 0.59 |
| ATOM | 97 | HB2 | LYS | 11 | −15.547 | 1.618 | −4.135 | 1.00 | 0.56 |
| ATOM | 98 | CG | LYS | 11 | −16.077 | 3.690 | −4.348 | 1.00 | 0.63 |
| ATOM | 99 | HG1 | LYS | 11 | −15.820 | 3.597 | −5.391 | 1.00 | 0.93 |
| ATOM | 100 | HG2 | LYS | 11 | −15.705 | 4.631 | −3.967 | 1.00 | 1.22 |
| ATOM | 101 | CD | LYS | 11 | −17.599 | 3.646 | −4.195 | 1.00 | 1.17 |
| ATOM | 102 | HD1 | LYS | 11 | −17.870 | 4.006 | −3.214 | 1.00 | 1.77 |
| ATOM | 103 | HD2 | LYS | 11 | −17.942 | 2.628 | −4.311 | 1.00 | 1.80 |
| ATOM | 104 | CE | LYS | 11 | −18.255 | 4.531 | −5.257 | 1.00 | 1.66 |
| ATOM | 105 | HE1 | LYS | 11 | −19.306 | 4.640 | −5.034 | 1.00 | 2.16 |
| ATOM | 106 | HE2 | LYS | 11 | −18.140 | 4.073 | −6.228 | 1.00 | 2.08 |
| ATOM | 107 | NZ | LYS | 11 | −17.608 | 5.874 | −5.261 | 1.00 | 2.39 |
| ATOM | 108 | HZ1 | LYS | 11 | −16.668 | 5.805 | −5.699 | 1.00 | 2.84 |
| ATOM | 109 | HZ2 | LYS | 11 | −17.512 | 6.215 | −4.282 | 1.00 | 2.78 |
| ATOM | 110 | HZ3 | LYS | 11 | −18.193 | 6.539 | −5.805 | 1.00 | 2.81 |
| ATOM | 111 | C | LYS | 11 | −13.432 | 1.788 | −2.279 | 1.00 | 0.48 |
| ATOM | 112 | O | LYS | 11 | −13.402 | 2.035 | −1.090 | 1.00 | 0.50 |
| ATOM | 113 | N | LYS | 12 | −12.989 | 0.650 | −2.743 | 1.00 | 0.47 |
| ATOM | 114 | HN | LYS | 12 | −13.009 | 0.470 | −3.705 | 1.00 | 0.47 |
| ATOM | 115 | CA | LYS | 12 | −12.436 | −0.366 | −1.802 | 1.00 | 0.49 |
| ATOM | 116 | HA | LYS | 12 | −13.193 | −0.650 | −1.084 | 1.00 | 0.55 |
| ATOM | 117 | CB | LYS | 12 | −11.966 | −1.601 | −2.576 | 1.00 | 0.51 |
| ATOM | 118 | HB1 | LYS | 12 | −11.411 | −2.249 | −1.913 | 1.00 | 0.52 |
| ATOM | 119 | HB2 | LYS | 12 | −11.328 | −1.292 | −3.390 | 1.00 | 0.51 |
| ATOM | 120 | CG | LYS | 12 | −13.169 | −2.363 | −3.135 | 1.00 | 0.64 |
| ATOM | 121 | HG1 | LYS | 12 | −13.724 | −1.723 | −3.803 | 1.00 | 1.06 |
| ATOM | 122 | HG2 | LYS | 12 | −13.807 | −2.679 | −2.321 | 1.00 | 1.06 |
| ATOM | 123 | CD | LYS | 12 | −12.669 | −3.590 | −3.902 | 1.00 | 0.82 |
| ATOM | 124 | HD1 | LYS | 12 | −12.111 | −4.229 | −3.234 | 1.00 | 1.47 |
| ATOM | 125 | HD2 | LYS | 12 | −12.029 | −3.270 | −4.711 | 1.00 | 1.51 |
| ATOM | 126 | CE | LYS | 12 | −13.858 | −4.367 | −4.469 | 1.00 | 1.39 |
| ATOM | 127 | HE1 | LYS | 12 | −13.611 | −4.733 | −5.455 | 1.00 | 1.89 |
| ATOM | 128 | HE2 | LYS | 12 | −14.718 | −3.717 | −4.531 | 1.00 | 1.86 |
| ATOM | 129 | NZ | LYS | 12 | −14.168 | −5.517 | −3.574 | 1.00 | 2.37 |
| ATOM | 130 | HZ1 | LYS | 12 | −14.867 | −5.226 | −2.863 | 1.00 | 2.74 |
| ATOM | 131 | HZ2 | LYS | 12 | −13.297 | −5.829 | −3.097 | 1.00 | 2.90 |
| ATOM | 132 | HZ3 | LYS | 12 | −14.555 | −6.301 | −4.137 | 1.00 | 2.83 |
| ATOM | 133 | C | LYS | 12 | −11.244 | 0.252 | −1.076 | 1.00 | 0.43 |
| ATOM | 134 | O | LYS | 12 | −11.026 | 0.019 | 0.097 | 1.00 | 0.45 |
| ATOM | 135 | N | TRP | 13 | −10.476 | 1.049 | −1.766 | 1.00 | 0.39 |
| ATOM | 136 | HN | TRP | 13 | −10.677 | 1.228 | −2.708 | 1.00 | 0.41 |
| ATOM | 137 | CA | TRP | 13 | −9.305 | 1.696 | −1.119 | 1.00 | 0.37 |
| ATOM | 138 | HA | TRP | 13 | −8.624 | 0.943 | −0.750 | 1.00 | 0.37 |
| ATOM | 139 | CB | TRP | 13 | −8.587 | 2.598 | −2.125 | 1.00 | 0.38 |
| ATOM | 140 | HB1 | TRP | 13 | −7.711 | 3.023 | −1.656 | 1.00 | 0.41 |
| ATOM | 141 | HB2 | TRP | 13 | −9.248 | 3.395 | −2.426 | 1.00 | 0.39 |
| ATOM | 142 | CG | TRP | 13 | −8.172 | 1.813 | −3.333 | 1.00 | 0.41 |
| ATOM | 143 | CD1 | TRP | 13 | −8.276 | 0.468 | −3.480 | 1.00 | 0.44 |
| ATOM | 144 | HD1 | TRP | 13 | −8.672 | −0.220 | −2.749 | 1.00 | 0.44 |
| ATOM | 145 | CD2 | TRP | 13 | −7.587 | 2.312 | −4.571 | 1.00 | 0.47 |
| ATOM | 146 | NE1 | TRP | 13 | −7.788 | 0.117 | −4.726 | 1.00 | 0.50 |
| ATOM | 147 | HE1 | TRP | 13 | −7.748 | −0.796 | −5.080 | 1.00 | 0.54 |
| ATOM | 148 | CE2 | TRP | 13 | −7.353 | 1.217 | −5.435 | 1.00 | 0.52 |
| ATOM | 149 | CE3 | TRP | 13 | −7.240 | 3.599 | −5.022 | 1.00 | 0.51 |
| ATOM | 150 | HE3 | TRP | 13 | −7.408 | 4.454 | −4.384 | 1.00 | 0.50 |
| ATOM | 151 | CZ2 | TRP | 13 | −6.793 | 1.392 | −6.702 | 1.00 | 0.61 |
| ATOM | 152 | HZ2 | TRP | 13 | −6.623 | 0.540 | −7.343 | 1.00 | 0.67 |
| ATOM | 153 | CZ3 | TRP | 13 | −6.677 | 3.779 | −6.296 | 1.00 | 0.60 |
| ATOM | 154 | HZ3 | TRP | 13 | −6.414 | 4.771 | −6.632 | 1.00 | 0.65 |
| ATOM | 155 | CH2 | TRP | 13 | −6.454 | 2.677 | −7.134 | 1.00 | 0.65 |
| ATOM | 156 | HH2 | TRP | 13 | 6.021 | 2.821 | −8.112 | 1.00 | 0.73 |
| ATOM | 157 | C | TRP | 13 | −9.803 | 2.548 | 0.044 | 1.00 | 0.38 |
| ATOM | 158 | O | TRP | 13 | −9.119 | 2.739 | 1.021 | 1.00 | 0.39 |
| ATOM | 159 | N | ALA | 14 | −10.994 | 3.061 | −0.050 | 1.00 | 0.42 |
| ATOM | 160 | HN | ALA | 14 | −11.540 | 2.896 | −0.847 | 1.00 | 0.45 |
| ATOM | 161 | CA | ALA | 14 | −11.527 | 3.892 | 1.062 | 1.00 | 0.48 |
| ATOM | 162 | HA | ALA | 14 | −10.739 | 4.509 | 1.466 | 1.00 | 0.48 |
| ATOM | 163 | CB | ALA | 14 | −12.658 | 4.779 | 0.536 | 1.00 | 0.55 |
| ATOM | 164 | HB1 | ALA | 14 | −12.555 | 5.774 | 0.943 | 1.00 | 1.11 |
| ATOM | 165 | HB2 | ALA | 14 | −13.610 | 4.365 | 0.835 | 1.00 | 1.21 |
| ATOM | 166 | HB3 | ALA | 14 | −12.608 | 4.824 | −0.542 | 1.00 | 1.14 |
| ATOM | 167 | C | ALA | 14 | −12.072 | 2.970 | 2.156 | 1.00 | 0.49 |
| ATOM | 168 | O | ALA | 14 | −12.450 | 3.412 | 3.223 | 1.00 | 0.55 |
| ATOM | 169 | N | GLU | 15 | −12.134 | 1.691 | 1.886 | 1.00 | 0.47 |
| ATOM | 170 | HN | GLU | 15 | −11.839 | 1.362 | 1.011 | 1.00 | 0.45 |
| ATOM | 171 | CA | GLU | 15 | −12.679 | 0.736 | 2.894 | 1.00 | 0.52 |
| ATOM | 172 | HA | GLU | 15 | −13.328 | 1.268 | 3.572 | 1.00 | 0.58 |
| ATOM | 173 | CB | GLU | 15 | −13.494 | −0.334 | 2.168 | 1.00 | 0.58 |
| ATOM | 174 | HB1 | GLU | 15 | −13.766 | −1.114 | 2.863 | 1.00 | 0.64 |
| ATOM | 175 | HB2 | GLU | 15 | −12.904 | −0.753 | 1.365 | 1.00 | 0.55 |
| ATOM | 176 | CO | GLU | 15 | −14.762 | 0.302 | 1.596 | 1.00 | 0.70 |
| ATOM | 177 | HG1 | GLU | 15 | −14.491 | 1.108 | 0.930 | 1.00 | 1.24 |
| ATOM | 178 | HG2 | GLU | 15 | −15.365 | 0.691 | 2.404 | 1.00 | 1.12 |
| ATOM | 179 | CD | GLU | 15 | −15.560 | −0.748 | 0.823 | 1.00 | 1.35 |
| ATOM | 180 | OE1 | GLU | 15 | −16.609 | −0.403 | 0.305 | 1.00 | 1.94 |
| ATOM | 181 | OE2 | GLU | 15 | −15.108 | −1.880 | 0.763 | 1.00 | 2.17 |
| ATOM | 182 | C | GLU | 15 | −11.556 | 0.062 | 3.698 | 1.00 | 0.47 |
| ATOM | 183 | O | GLU | 15 | −11.765 | −0.345 | 4.824 | 1.00 | 0.51 |
| ATOM | 184 | N | SER | 16 | −10.375 | −0.080 | 3.150 | 1.00 | 0.42 |
| ATOM | 185 | HN | SER | 16 | −10.207 | 0.238 | 2.238 | 1.00 | 0.42 |
| ATOM | 186 | CA | SER | 16 | −9.291 | −0.751 | 3.933 | 1.00 | 0.42 |
| ATOM | 187 | HA | SER | 16 | −9.188 | −0.259 | 4.889 | 1.00 | 0.44 |
| ATOM | 188 | CB | SER | 16 | −9.670 | −2.214 | 4.165 | 1.00 | 0.48 |
| ATOM | 189 | HB1 | SER | 16 | −10.416 | −2.272 | 4.947 | 1.00 | 0.54 |
| ATOM | 190 | HB2 | SER | 16 | −8.798 | −2.771 | 4.462 | 1.00 | 0.50 |
| ATOM | 191 | OG | SER | 16 | −10.186 | −2.761 | 2.959 | 1.00 | 0.53 |
| ATOM | 192 | HG | SER | 16 | −9.619 | −2.476 | 2.238 | 1.00 | 0.96 |
| ATOM | 193 | C | SER | 16 | −7.949 | −0.693 | 3.192 | 1.00 | 0.38 |
| ATOM | 194 | O | SER | 16 | −7.879 | −0.820 | 1.986 | 1.00 | 0.37 |
| ATOM | 195 | N | LEU | 17 | −6.884 | −0.511 | 3.930 | 1.00 | 0.37 |
| ATOM | 196 | HN | LEU | 17 | −6.984 | −0.421 | 4.901 | 1.00 | 0.40 |
| ATOM | 197 | CA | LEU | 17 | −5.518 | −0.442 | 3.327 | 1.00 | 0.37 |
| ATOM | 198 | HA | LEU | 17 | −5.470 | 0.385 | 2.639 | 1.00 | 0.36 |
| ATOM | 199 | CB | LEU | 17 | −4.507 | −0.218 | 4.456 | 1.00 | 0.40 |
| ATOM | 200 | HB1 | LEU | 17 | −4.570 | −1.038 | 5.156 | 1.00 | 0.44 |
| ATOM | 201 | HB2 | LEU | 17 | −4.743 | 0.704 | 4.967 | 1.00 | 0.43 |
| ATOM | 202 | CG | LEU | 17 | −3.082 | −0.138 | 3.902 | 1.00 | 0.45 |
| ATOM | 203 | HG | LEU | 17 | −2.848 | −1.047 | 3.375 | 1.00 | 0.54 |
| ATOM | 204 | CD1 | LEU | 17 | −2.945 | 1.053 | 2.952 | 1.00 | 0.57 |
| ATOM | 205 | HD11 | LEU | 17 | −3.281 | 0.771 | 1.966 | 1.00 | 1.30 |
| ATOM | 206 | HD12 | LEU | 17 | −1.909 | 1.356 | 2.905 | 1.00 | 1.08 |
| ATOM | 207 | HD13 | LEU | 17 | −3.542 | 1.874 | 3.318 | 1.00 | 1.17 |
| ATOM | 208 | CD2 | LEU | 17 | −2.109 | 0.039 | 5.066 | 1.00 | 0.49 |
| ATOM | 209 | HD21 | LEU | 17 | −2.302 | −0.716 | 5.809 | 1.00 | 1.13 |
| ATOM | 210 | HD22 | LEU | 17 | −2.248 | 1.014 | 5.503 | 1.00 | 1.06 |
| ATOM | 211 | HD23 | LEU | 17 | −1.095 | −0.057 | 4.708 | 1.00 | 1.17 |
| ATOM | 212 | C | LEU | 17 | −5.184 | −1.744 | 2.587 | 1.00 | 0.38 |
| ATOM | 213 | O | LEU | 17 | −4.592 | −1.725 | 1.526 | 1.00 | 0.39 |
| ATOM | 214 | N | GLU | 18 | −5.533 | −2.872 | 3.141 | 1.00 | 0.40 |
| ATOM | 215 | HN | GLU | 18 | −5.986 | −2.874 | 4.007 | 1.00 | 0.41 |
| ATOM | 216 | CA | GLU | 18 | −5.205 | −4.162 | 2.470 | 1.00 | 0.43 |
| ATOM | 217 | HA | GLU | 18 | −4.136 | −4.299 | 2.461 | 1.00 | 0.45 |
| ATOM | 218 | CB | GLU | 18 | −5.860 | −5.314 | 3.239 | 1.00 | 0.49 |
| ATOM | 219 | HB1 | GLU | 18 | −5.772 | −6.225 | 2.665 | 1.00 | 0.53 |
| ATOM | 220 | HB2 | GLU | 18 | −6.904 | −5.091 | 3.402 | 1.00 | 0.48 |
| ATOM | 221 | CG | GLU | 18 | −5.159 | −5.495 | 4.587 | 1.00 | 0.53 |
| ATOM | 222 | HG1 | GLU | 18 | −5.243 | −4.586 | 5.163 | 1.00 | 0.76 |
| ATOM | 223 | HG2 | GLU | 18 | −4.116 | −5.721 | 4.419 | 1.00 | 0.70 |
| ATOM | 224 | CD | GLU | 18 | −5.812 | −6.646 | 5.355 | 1.00 | 0.92 |
| ATOM | 225 | OE1 | GLU | 18 | −6.853 | −7.108 | 4.920 | 1.00 | 1.64 |
| ATOM | 226 | OE2 | GLU | 18 | −5.260 | −7.044 | 6.368 | 1.00 | 1.49 |
| ATOM | 227 | C | GLU | 18 | −5.730 | −4.151 | 1.034 | 1.00 | 0.42 |
| ATOM | 228 | O | GLU | 18 | −5.077 | −4.625 | 0.126 | 1.00 | 0.44 |
| ATOM | 229 | N | ASN | 19 | −6.896 | −3.618 | 0.813 | 1.00 | 0.41 |
| ATOM | 230 | HN | ASN | 19 | −7.412 | −3.238 | 1.554 | 1.00 | 0.41 |
| ATOM | 231 | CA | ASN | 19 | −7.439 | −3.588 | −0.575 | 1.00 | 0.41 |
| ATOM | 232 | HA | ASN | 19 | −7.504 | −4.595 | −0.959 | 1.00 | 0.45 |
| ATOM | 233 | CB | ASN | 19 | −8.832 | −2.957 | −0.560 | 1.00 | 0.42 |
| ATOM | 234 | HB1 | ASN | 19 | −9.146 | −2.755 | −1.573 | 1.00 | 0.42 |
| ATOM | 235 | HB2 | ASN | 19 | −8.801 | −2.032 | −0.001 | 1.00 | 0.41 |
| ATOM | 236 | CG | ASN | 19 | −9.822 | −3.918 | 0.097 | 1.00 | 0.48 |
| ATOM | 237 | OD1 | ASN | 19 | −9.650 | −4.301 | 1.237 | 1.00 | 1.22 |
| ATOM | 238 | ND2 | ASN | 19 | −10.862 | −4.324 | −0.578 | 1.00 | 1.19 |
| ATOM | 239 | HD21 | ASN | 19 | −11.003 | −4.011 | −1.496 | 1.00 | 1.98 |
| ATOM | 240 | HD22 | ASN | 19 | −11.503 | −4.941 | −0.167 | 1.00 | 1.21 |
| ATOM | 241 | C | ASN | 19 | −6.518 | −2.755 | −1.470 | 1.00 | 0.38 |
| ATOM | 242 | O | ASN | 19 | −6.220 | −3.127 | −2.589 | 1.00 | 0.40 |
| ATOM | 243 | N | LEU | 20 | −6.061 | −1.633 | −0.986 | 1.00 | 0.35 |
| ATOM | 244 | HN | LEU | 20 | −6.312 | −1.353 | −0.080 | 1.00 | 0.35 |
| ATOM | 245 | CA | LEU | 20 | −5.159 | −0.777 | −1.807 | 1.00 | 0.34 |
| ATOM | 246 | HA | LEU | 20 | −5.655 | −0.512 | −2.728 | 1.00 | 0.37 |
| ATOM | 247 | CB | LEU | 20 | −4.827 | 0.494 | −1.013 | 1.00 | 0.34 |
| ATOM | 248 | HB1 | LEU | 20 | −4.340 | 0.215 | −0.090 | 1.00 | 0.35 |
| ATOM | 249 | HB2 | LEU | 20 | −5.742 | 1.019 | −0.783 | 1.00 | 0.37 |
| ATOM | 250 | CG | LEU | 20 | −3.896 | 1.418 | −1.814 | 1.00 | 0.35 |
| ATOM | 251 | HG | LEU | 20 | −3.027 | 0.870 | −2.147 | 1.00 | 0.38 |
| ATOM | 252 | CD1 | LEU | 20 | −4.633 | 1.986 | −3.032 | 1.00 | 0.39 |
| ATOM | 253 | HD11 | LEU | 20 | −4.627 | 1.260 | −3.828 | 1.00 | 1.05 |
| ATOM | 254 | HD12 | LEU | 20 | −4.136 | 2.885 | −3.365 | 1.00 | 1.12 |
| ATOM | 255 | HD13 | LEU | 20 | −5.653 | 2.219 | −2.765 | 1.00 | 1.10 |
| ATOM | 256 | CD2 | LEU | 20 | −3.453 | 2.573 | −0.913 | 1.00 | 0.38 |
| ATOM | 257 | HD21 | LEU | 20 | −2.688 | 3.147 | −1.412 | 1.00 | 0.99 |
| ATOM | 258 | HD22 | LEU | 20 | −3.061 | 2.177 | 0.012 | 1.00 | 1.12 |
| ATOM | 259 | HD23 | LEU | 20 | −4.300 | 3.210 | −0.702 | 1.00 | 1.05 |
| ATOM | 260 | C | LEU | 20 | −3.877 | −1.558 | −2.117 | 1.00 | 0.36 |
| ATOM | 261 | O | LEU | 20 | −3.353 | −1.506 | −3.212 | 1.00 | 0.41 |
| ATOM | 262 | N | ILE | 21 | −3.373 | −2.284 | −1.156 | 1.00 | 0.36 |
| ATOM | 263 | HN | ILE | 21 | −3.816 | −2.310 | −0.282 | 1.00 | 0.36 |
| ATOM | 264 | CA | ILE | 21 | −2.129 | −3.075 | −1.379 | 1.00 | 0.41 |
| ATOM | 265 | HA | ILE | 21 | −1.429 | −2.488 | −1.955 | 1.00 | 0.43 |
| ATOM | 266 | CB | ILE | 21 | −1.505 | −3.430 | −0.013 | 1.00 | 0.47 |
| ATOM | 267 | HB | ILE | 21 | −2.206 | −4.021 | 0.554 | 1.00 | 1.27 |
| ATOM | 268 | CG1 | ILE | 21 | −1.189 | −2.148 | 0.759 | 1.00 | 1.31 |
| ATOM | 269 | HG11 | ILE | 21 | −2.105 | −1.621 | 0.977 | 1.00 | 2.03 |
| ATOM | 270 | HG12 | ILE | 21 | −0.541 | −1.518 | 0.166 | 1.00 | 1.83 |
| ATOM | 271 | CG2 | ILE | 21 | −0.199 | −4.221 | −0.176 | 1.00 | 1.28 |
| ATOM | 272 | HG21 | ILE | 21 | −0.199 | −4.759 | −1.105 | 1.00 | 1.89 |
| ATOM | 273 | HG22 | ILE | 21 | −0.105 | −4.922 | 0.641 | 1.00 | 1.82 |
| ATOM | 274 | HG23 | ILE | 21 | −0.636 | −3.538 | −0.159 | 1.00 | 1.86 |
| ATOM | 275 | CD1 | ILE | 21 | −0.490 | −2.517 | 2.070 | 1.00 | 1.34 |
| ATOM | 276 | HD11 | ILE | 21 | −0.759 | −1.812 | 2.837 | 1.00 | 1.53 |
| ATOM | 277 | HD12 | ILE | 21 | −0.581 | −2.499 | 1.926 | 1.00 | 1.73 |
| ATOM | 278 | HD13 | ILE | 21 | −0.796 | −3.505 | 2.372 | 1.00 | 1.71 |
| ATOM | 279 | C | ILE | 21 | −2.480 | −4.358 | −2.148 | 1.00 | 0.44 |
| ATOM | 280 | O | ILE | 21 | −1.670 | −4.901 | −2.874 | 1.00 | 0.47 |
| ATOM | 281 | N | ASN | 22 | −3.671 | −4.861 | −1.981 | 1.00 | 0.44 |
| ATOM | 282 | HN | ASN | 22 | −4.310 | −4.423 | −1.382 | 1.00 | 0.43 |
| ATOM | 283 | CA | ASN | 22 | −4.044 | −6.125 | −2.681 | 1.00 | 0.48 |
| ATOM | 284 | HA | ASN | 22 | −3.166 | −6.745 | −2.779 | 1.00 | 0.52 |
| ATOM | 285 | CB | ASN | 22 | −5.089 | −6.872 | −1.850 | 1.00 | 0.53 |
| ATOM | 286 | HB1 | ASN | 22 | −5.476 | −7.703 | −2.420 | 1.00 | 0.58 |
| ATOM | 287 | HB2 | ASN | 22 | −5.897 | −6.199 | −1.599 | 1.00 | 0.51 |
| ATOM | 288 | CG | ASN | 22 | −4.440 | −7.394 | −0.567 | 1.00 | 0.59 |
| ATOM | 289 | OD1 | ASN | 22 | −3.376 | −7.980 | −0.605 | 1.00 | 1.15 |
| ATOM | 290 | ND2 | ASN | 22 | −5.040 | −7.206 | 0.576 | 1.00 | 1.33 |
| ATOM | 291 | HD21 | ASN | 22 | −5.898 | −6.732 | 0.607 | 1.00 | 2.10 |
| ATOM | 292 | HD22 | ASN | 22 | −4.634 | −7.538 | 1.403 | 1.00 | 1.37 |
| ATOM | 293 | C | ASN | 22 | −4.612 | −5.840 | −4.075 | 1.00 | 0.46 |
| ATOM | 294 | O | ASN | 22 | −4.958 | −6.752 | −4.800 | 1.00 | 0.50 |
| ATOM | 295 | N | HIS | 23 | −4.701 | −4.598 | −4.470 | 1.00 | 0.42 |
| ATOM | 296 | HN | HIS | 23 | −4.410 | −3.871 | −3.880 | 1.00 | 0.41 |
| ATOM | 297 | CA | HIS | 23 | −5.236 | −4.292 | −5.831 | 1.00 | 0.44 |
| ATOM | 298 | HA | HIS | 23 | −5.874 | −5.101 | −6.157 | 1.00 | 0.48 |
| ATOM | 299 | CB | HIS | 23 | −6.043 | −2.995 | −5.795 | 1.00 | 0.44 |
| ATOM | 300 | HB1 | HIS | 23 | −5.376 | −2.159 | −5.684 | 1.00 | 0.42 |
| ATOM | 301 | HB2 | HIS | 23 | −6.730 | −3.021 | −4.962 | 1.00 | 0.46 |
| ATOM | 302 | CG | HIS | 23 | −6.814 | −2.856 | −7.078 | 1.00 | 0.54 |
| ATOM | 303 | ND1 | HIS | 23 | −6.275 | −2.251 | −8.203 | 1.00 | 0.89 |
| ATOM | 304 | HD1 | HIS | 23 | −5.378 | −1.863 | −8.278 | 1.00 | 1.50 |
| ATOM | 305 | CD2 | HIS | 23 | −8.081 | −3.246 | −7.433 | 1.00 | 1.13 |
| ATOM | 306 | HD2 | HIS | 23 | −8.781 | −3.751 | −6.784 | 1.00 | 1.83 |
| ATOM | 307 | CE1 | HIS | 23 | −7.207 | −2.294 | −9.172 | 1.00 | 0.77 |
| ATOM | 308 | HE1 | HIS | 23 | −7.066 | −1.897 | −10.166 | 1.00 | 1.14 |
| ATOM | 309 | NE2 | HIS | 23 | −8.328 | −2.890 | −8.756 | 1.00 | 0.94 |
| ATOM | 310 | C | HIS | 23 | −4.063 | −4.148 | −6.808 | 1.00 | 0.45 |
| ATOM | 311 | O | HIS | 23 | −3.062 | −3.529 | −6.506 | 1.00 | 0.41 |
| ATOM | 312 | N | GLU | 24 | −4.171 | −4.741 | −7.968 | 1.00 | 0.54 |
| ATOM | 313 | HN | GLU | 24 | −4.980 | −5.251 | −8.180 | 1.00 | 0.58 |
| ATOM | 314 | CA | GLU | 24 | −3.056 | −4.671 | −8.960 | 1.00 | 0.59 |
| ATOM | 315 | HA | GLU | 24 | −2.168 | −5.109 | −8.529 | 1.00 | 0.60 |
| ATOM | 316 | CB | GLU | 24 | −3.447 | −5.458 | −10.214 | 1.00 | 0.72 |
| ATOM | 317 | HB1 | GLU | 24 | −4.340 | −5.028 | −10.642 | 1.00 | 0.74 |
| ATOM | 318 | HB2 | GLU | 24 | −3.635 | −6.487 | −9.947 | 1.00 | 0.76 |
| ATOM | 319 | CG | GLU | 24 | −2.312 | −5.394 | −11.240 | 1.00 | 0.78 |
| ATOM | 320 | HG1 | GLU | 24 | −1.390 | −5.715 | −10.778 | 1.00 | 1.02 |
| ATOM | 321 | HG2 | GLU | 24 | −2.204 | −4.380 | −11.596 | 1.00 | 1.07 |
| ATOM | 322 | CD | GLU | 24 | −2.637 | −6.315 | −12.417 | 1.00 | 1.39 |
| ATOM | 323 | OE1 | GLU | 24 | −3.745 | −6.232 | −12.921 | 1.00 | 1.95 |
| ATOM | 324 | OE2 | GLU | 24 | −1.774 | −7.093 | −12.791 | 1.00 | 2.12 |
| ATOM | 325 | C | GLU | 24 | −2.764 | −3.216 | −9.343 | 1.00 | 0.56 |
| ATOM | 326 | O | GLU | 24 | −1.620 | −2.822 | −9.488 | 1.00 | 0.57 |
| ATOM | 327 | N | CYS | 25 | −3.773 | −2.406 | −9.507 | 1.00 | 0.56 |
| ATOM | 328 | HN | CYS | 25 | −4.692 | −2.727 | −9.389 | 1.00 | 0.57 |
| ATOM | 329 | CA | CYS | 25 | −3.512 | −0.991 | −9.885 | 1.00 | 0.58 |
| ATOM | 330 | HA | CYS | 25 | −2.650 | −0.947 | −10.536 | 1.00 | 0.62 |
| ATOM | 331 | CB | CYS | 25 | −4.725 | −0.402 | −10.606 | 1.00 | 0.66 |
| ATOM | 332 | HB1 | CYS | 25 | −5.469 | −0.107 | −9.881 | 1.00 | 0.59 |
| ATOM | 333 | HB2 | CYS | 25 | −5.143 | −1.142 | −11.273 | 1.00 | 0.74 |
| ATOM | 334 | SG | CYS | 25 | −4.206 | 1.045 | −11.562 | 1.00 | 0.90 |
| ATOM | 335 | HG | CYS | 25 | −4.416 | 1.830 | −11.051 | 1.00 | 1.39 |
| ATOM | 336 | C | CYS | 25 | −3.227 | −0.193 | −8.619 | 1.00 | 0.49 |
| ATOM | 337 | O | CYS | 25 | −2.470 | 0.757 | −8.631 | 1.00 | 0.50 |
| ATOM | 338 | N | GLY | 26 | −3.796 | −0.588 | −7.514 | 1.00 | 0.44 |
| ATOM | 339 | HN | GLY | 26 | −4.384 | −1.372 | −7.510 | 1.00 | 0.46 |
| ATOM | 340 | CA | GLY | 26 | −3.507 | 0.141 | −6.253 | 1.00 | 0.39 |
| ATOM | 341 | HA1 | GLY | 26 | −4.035 | −0.312 | −5.433 | 1.00 | 0.38 |
| ATOM | 342 | HA2 | GLY | 26 | −3.799 | 1.178 | −6.354 | 1.00 | 0.44 |
| ATOM | 343 | C | GLY | 26 | −2.007 | 0.042 | −6.009 | 1.00 | 0.36 |
| ATOM | 344 | O | GLY | 26 | −1.351 | 1.009 | −5.687 | 1.00 | 0.37 |
| ATOM | 345 | N | LEU | 27 | −1.455 | −1.127 | −6.196 | 1.00 | 0.38 |
| ATOM | 346 | HN | LEU | 27 | −2.003 | −1.889 | −6.480 | 1.00 | 0.40 |
| ATOM | 347 | CA | LEU | 27 | 0.010 | −1.292 | −6.016 | 1.00 | 0.40 |
| ATOM | 348 | HA | LEU | 27 | 0.283 | −1.029 | −5.006 | 1.00 | 0.38 |
| ATOM | 349 | CB | LEU | 27 | 0.401 | −2.746 | −6.304 | 1.00 | 0.47 |
| ATOM | 350 | HB1 | LEU | 27 | 1.474 | −2.821 | −6.397 | 1.00 | 0.54 |
| ATOM | 351 | HB2 | LEU | 27 | −0.060 | −3.060 | −7.230 | 1.00 | 0.51 |
| ATOM | 352 | CG | LEU | 27 | −0.081 | −3.653 | −5.165 | 1.00 | 0.45 |
| ATOM | 353 | HG | LEU | 27 | −1.060 | −3.332 | −4.839 | 1.00 | 0.42 |
| ATOM | 354 | CD1 | LEU | 27 | −0.158 | −5.096 | −5.665 | 1.00 | 0.52 |
| ATOM | 355 | HD11 | LEU | 27 | 0.780 | −5.368 | −6.127 | 1.00 | 1.23 |
| ATOM | 356 | HD12 | LEU | 27 | −0.955 | −5.185 | −6.389 | 1.00 | 1.16 |
| ATOM | 357 | HD13 | LEU | 27 | −0.353 | −5.756 | −4.832 | 1.00 | 1.05 |
| ATOM | 358 | CD2 | LEU | 27 | 0.902 | −3.589 | −3.988 | 1.00 | 0.48 |
| ATOM | 359 | HD21 | LEU | 27 | 1.549 | −2.732 | −4.093 | 1.00 | 1.12 |
| ATOM | 360 | HD22 | LEU | 27 | 1.500 | −4.488 | −3.973 | 1.00 | 1.17 |
| ATOM | 361 | HD23 | LEU | 27 | 0.350 | −3.512 | −3.064 | 1.00 | 1.06 |
| ATOM | 362 | C | LEU | 27 | 0.720 | −0.364 | −7.000 | 1.00 | 0.43 |
| ATOM | 363 | O | LEU | 27 | 1.618 | 0.371 | −6.641 | 1.00 | 0.43 |
| ATOM | 364 | N | ALA | 28 | 0.310 | −0.384 | −8.242 | 1.00 | 0.48 |
| ATOM | 365 | HN | ALA | 28 | −0.424 | −0.981 | −8.510 | 1.00 | 0.49 |
| ATOM | 366 | CA | ALA | 28 | 0.952 | 0.508 | −9.248 | 1.00 | 0.53 |
| ATOM | 367 | HA | ALA | 28 | 2.004 | 0.281 | −9.312 | 1.00 | 0.56 |
| ATOM | 368 | CB | ALA | 28 | 0.298 | 0.297 | −10.615 | 1.00 | 0.61 |
| ATOM | 369 | HB1 | ALA | 28 | −0.756 | 0.525 | −10.549 | 1.00 | 1.18 |
| ATOM | 370 | HB2 | ALA | 28 | 0.425 | −0.731 | −10.920 | 1.00 | 1.16 |
| ATOM | 371 | HB3 | ALA | 28 | 0.762 | 0.948 | −11.340 | 1.00 | 1.21 |
| ATOM | 372 | C | ALA | 28 | 0.770 | 1.964 | −8.819 | 1.00 | 0.50 |
| ATOM | 373 | O | ALA | 28 | 1.709 | 2.736 | −8.784 | 1.00 | 0.50 |
| ATOM | 374 | N | ALA | 29 | −0.433 | 2.344 | −8.491 | 1.00 | 0.49 |
| ATOM | 375 | HN | ALA | 29 | −1.173 | 1.703 | −8.526 | 1.00 | 0.50 |
| ATOM | 376 | CA | ALA | 29 | −0.684 | 3.748 | −8.063 | 1.00 | 0.49 |
| ATOM | 377 | HA | ALA | 29 | −0.283 | 4.429 | −8.799 | 1.00 | 0.53 |
| ATOM | 378 | CB | ALA | 29 | −2.192 | 3.974 | −7.924 | 1.00 | 0.52 |
| ATOM | 379 | HB1 | ALA | 29 | −2.496 | 3.756 | −6.910 | 1.00 | 1.18 |
| ATOM | 380 | HB2 | ALA | 29 | −2.719 | 3.322 | −8.605 | 1.00 | 1.21 |
| ATOM | 381 | HB3 | ALA | 29 | −2.426 | 5.002 | −8.156 | 1.00 | 1.00 |
| ATOM | 382 | C | ALA | 29 | −0.015 | 3.997 | −6.712 | 1.00 | 0.43 |
| ATOM | 383 | O | ALA | 29 | 0.552 | 5.044 | −6.471 | 1.00 | 0.45 |
| ATOM | 384 | N | PHE | 30 | −0.089 | 3.045 | −5.824 | 1.00 | 0.39 |
| ATOM | 385 | HN | PHE | 30 | −0.558 | 2.212 | −6.038 | 1.00 | 0.40 |
| ATOM | 386 | CA | PHE | 30 | 0.530 | 3.227 | −4.484 | 1.00 | 0.37 |
| ATOM | 387 | HA | PHE | 30 | 0.165 | 4.146 | −4.051 | 1.00 | 0.41 |
| ATOM | 388 | CB | PHE | 30 | 0.147 | 2.055 | −3.576 | 1.00 | 0.38 |
| ATOM | 389 | HB1 | PHE | 30 | 0.545 | 1.138 | −3.984 | 1.00 | 0.43 |
| ATOM | 390 | HB2 | PHE | 30 | −0.929 | 1.984 | −3.513 | 1.00 | 0.40 |
| ATOM | 391 | CG | PHE | 30 | 0.716 | 2.282 | −2.197 | 1.00 | 0.48 |
| ATOM | 392 | CD1 | PHE | 30 | 0.267 | 3.363 | −1.428 | 1.00 | 0.64 |
| ATOM | 393 | HD1 | PHE | 30 | −0.486 | 4.031 | −1.821 | 1.00 | 0.71 |
| ATOM | 394 | CD2 | PHE | 30 | 1.690 | 1.416 | −1.685 | 1.00 | 0.59 |
| ATOM | 395 | HD2 | PHE | 30 | 2.035 | 0.580 | −2.275 | 1.00 | 0.64 |
| ATOM | 396 | CE1 | PHE | 30 | 0.793 | 3.580 | −0.151 | 1.00 | 0.82 |
| ATOM | 397 | HE1 | PHE | 30 | 0.446 | 4.412 | 0.440 | 1.00 | 1.00 |
| ATOM | 398 | CE2 | PHE | 30 | 2.213 | 1.633 | −0.406 | 1.00 | 0.76 |
| ATOM | 399 | HE2 | PHE | 30 | 2.963 | 0.964 | −0.009 | 1.00 | 0.90 |
| ATOM | 400 | CZ | PHE | 30 | 1.765 | 2.715 | 0.360 | 1.00 | 0.86 |
| ATOM | 401 | HZ | PHE | 30 | 2.171 | 2.887 | 1.344 | 1.00 | 1.03 |
| ATOM | 402 | C | PHE | 30 | 2.054 | 3.309 | −4.615 | 1.00 | 0.36 |
| ATOM | 403 | O | PHE | 30 | 2.691 | 4.134 | −3.992 | 1.00 | 0.37 |
| ATOM | 404 | N | LYS | 31 | 2.650 | 2.468 | −5.421 | 1.00 | 0.38 |
| ATOM | 405 | HN | LYS | 31 | 2.126 | 1.808 | −5.922 | 1.00 | 0.40 |
| ATOM | 406 | CA | LYS | 31 | 4.132 | 2.527 | −5.573 | 1.00 | 0.40 |
| ATOM | 407 | HA | LYS | 31 | 4.598 | 2.360 | −4.613 | 1.00 | 0.41 |
| ATOM | 408 | CB | LYS | 31 | 4.607 | 1.457 | −6.562 | 1.00 | 0.47 |
| ATOM | 409 | HB1 | LYS | 31 | 5.586 | 1.721 | −6.932 | 1.00 | 0.52 |
| ATOM | 410 | HB2 | LYS | 31 | 3.914 | 1.402 | −7.389 | 1.00 | 0.49 |
| ATOM | 411 | CG | LYS | 31 | 4.680 | 0.094 | −5.869 | 1.00 | 0.51 |
| ATOM | 412 | HG1 | LYS | 31 | 3.699 | −0.184 | −5.513 | 1.00 | 0.80 |
| ATOM | 413 | HG2 | LYS | 31 | 5.364 | 0.157 | −5.035 | 1.00 | 0.72 |
| ATOM | 414 | CD | LYS | 31 | 5.173 | −0.953 | −6.878 | 1.00 | 0.73 |
| ATOM | 415 | HD1 | LYS | 31 | 6.052 | −0.577 | −7.380 | 1.00 | 1.40 |
| ATOM | 416 | HD2 | LYS | 31 | 4.398 | −1.137 | −7.607 | 1.00 | 1.34 |
| ATOM | 417 | CE | LYS | 31 | 5.521 | −2.270 | −6.168 | 1.00 | 1.20 |
| ATOM | 418 | HE1 | LYS | 31 | 5.814 | −2.074 | −5.148 | 1.00 | 1.85 |
| ATOM | 419 | HE2 | LYS | 31 | 6.339 | −2.748 | −6.687 | 1.00 | 1.74 |
| ATOM | 420 | NZ | LYS | 31 | 4.337 | −3.174 | −6.180 | 1.00 | 1.97 |
| ATOM | 421 | HZ1 | LYS | 31 | 4.363 | −3.788 | −5.342 | 1.00 | 2.46 |
| ATOM | 422 | HZ2 | LYS | 31 | 4.357 | −3.759 | −7.041 | 1.00 | 2.37 |
| ATOM | 423 | HZ3 | LYS | 31 | 3.467 | −2.607 | −6.165 | 1.00 | 2.48 |
| ATOM | 424 | C | LYS | 31 | 4.535 | 3.905 | −6.099 | 1.00 | 0.41 |
| ATOM | 425 | O | LYS | 31 | 5.483 | 4.509 | −5.632 | 1.00 | 0.42 |
| ATOM | 426 | N | ALA | 32 | 3.819 | 4.413 | −7.063 | 1.00 | 0.45 |
| ATOM | 427 | HN | ALA | 32 | 3.056 | 3.915 | −7.425 | 1.00 | 0.46 |
| ATOM | 428 | CA | ALA | 32 | 4.164 | 5.751 | −7.611 | 1.00 | 0.51 |
| ATOM | 429 | HA | ALA | 32 | 5.213 | 5.777 | −7.868 | 1.00 | 0.53 |
| ATOM | 430 | CB | ALA | 32 | 3.324 | 6.024 | −8.860 | 1.00 | 0.62 |
| ATOM | 431 | HB1 | ALA | 32 | 2.389 | 5.487 | −8.790 | 1.00 | 1.11 |
| ATOM | 432 | HB2 | ALA | 32 | 3.863 | 5.694 | −9.736 | 1.00 | 1.10 |
| ATOM | 433 | HB3 | ALA | 32 | 3.126 | 7.083 | −8.937 | 1.00 | 1.24 |
| ATOM | 434 | C | ALA | 32 | 3.872 | 6.814 | −6.554 | 1.00 | 0.48 |
| ATOM | 435 | O | ALA | 32 | 4.627 | 7.748 | −6.372 | 1.00 | 0.49 |
| ATOM | 436 | N | PHE | 33 | 2.784 | 6.676 | −5.847 | 1.00 | 0.48 |
| ATOM | 437 | HN | PHE | 33 | 2.189 | 5.912 | −6.003 | 1.00 | 0.49 |
| ATOM | 438 | CA | PHE | 33 | 2.454 | 7.675 | −4.796 | 1.00 | 0.53 |
| ATOM | 439 | HA | PHE | 33 | 2.365 | 8.648 | −5.253 | 1.00 | 0.60 |
| ATOM | 440 | CB | PHE | 33 | 1.133 | 7.316 | −4.120 | 1.00 | 0.60 |
| ATOM | 441 | HB1 | PHE | 33 | 1.205 | 6.334 | −3.677 | 1.00 | 0.56 |
| ATOM | 442 | HB2 | PHE | 33 | 0.335 | 7.331 | −4.848 | 1.00 | 0.66 |
| ATOM | 443 | CG | PHE | 33 | 0.863 | 8.336 | −3.046 | 1.00 | 0.76 |
| ATOM | 444 | CD1 | PHE | 33 | 0.371 | 9.600 | −3.389 | 1.00 | 0.93 |
| ATOM | 445 | HD1 | PHE | 33 | 0.170 | 9.841 | −4.422 | 1.00 | 0.97 |
| ATOM | 446 | CD2 | PHE | 33 | 1.125 | 8.023 | −1.708 | 1.00 | 0.84 |
| ATOM | 447 | HD2 | PHE | 33 | 1.502 | 7.046 | −1.446 | 1.00 | 0.82 |
| ATOM | 448 | CE1 | PHE | 33 | 0.143 | 10.554 | −2.391 | 1.00 | 1.11 |
| ATOM | 449 | HE1 | PHE | 33 | −0.236 | 11.530 | −2.653 | 1.00 | 1.27 |
| ATOM | 450 | CE2 | PHE | 33 | 0.893 | 8.974 | −0.711 | 1.00 | 1.05 |
| ATOM | 451 | HE2 | PHE | 33 | 1.093 | 8.733 | 0.323 | 1.00 | 1.16 |
| ATOM | 452 | CZ | PHE | 33 | 0.404 | 10.239 | −1.052 | 1.00 | 1.16 |
| ATOM | 453 | HZ | PHE | 33 | 0.233 | 10.974 | −0.283 | 1.00 | 1.33 |
| ATOM | 454 | C | PHE | 33 | 3.567 | 7.717 | −3.748 | 1.00 | 0.47 |
| ATOM | 455 | O | PHE | 33 | 3.965 | 8.770 | −3.297 | 1.00 | 0.52 |
| ATOM | 456 | N | LEU | 34 | 4.073 | 6.583 | −3.355 | 1.00 | 0.42 |
| ATOM | 457 | HN | LEU | 34 | 3.740 | 5.741 | −3.729 | 1.00 | 0.43 |
| ATOM | 458 | CA | LEU | 34 | 5.160 | 6.574 | −2.335 | 1.00 | 0.44 |
| ATOM | 459 | HA | LEU | 34 | 4.810 | 7.052 | −1.436 | 1.00 | 0.53 |
| ATOM | 460 | CB | LEU | 34 | 5.587 | 5.135 | −2.027 | 1.00 | 0.51 |
| ATOM | 461 | HB1 | LEU | 34 | 6.490 | 5.150 | −1.434 | 1.00 | 0.57 |
| ATOM | 462 | HB2 | LEU | 34 | 5.781 | 4.618 | −2.955 | 1.00 | 0.50 |
| ATOM | 463 | CG | LEU | 34 | 4.483 | 4.395 | −1.257 | 1.00 | 0.62 |
| ATOM | 464 | HG | LEU | 34 | 3.562 | 4.450 | −1.822 | 1.00 | 0.53 |
| ATOM | 465 | CD1 | LEU | 34 | 4.900 | 2.919 | −1.119 | 1.00 | 0.82 |
| ATOM | 466 | HD11 | LEU | 34 | 5.962 | 2.832 | −1.296 | 1.00 | 1.31 |
| ATOM | 467 | HD12 | LEU | 34 | 4.369 | 2.329 | −1.851 | 1.00 | 1.24 |
| ATOM | 468 | HD13 | LEU | 34 | 4.672 | 2.548 | −0.134 | 1.00 | 1.37 |
| ATOM | 469 | CD2 | LEU | 34 | 4.280 | 5.051 | 0.129 | 1.00 | 0.81 |
| ATOM | 470 | HD21 | LEU | 34 | 5.215 | 5.455 | 0.477 | 1.00 | 1.25 |
| ATOM | 471 | HD22 | LEU | 34 | 3.916 | 4.330 | 0.840 | 1.00 | 1.37 |
| ATOM | 472 | HD23 | LEU | 34 | 3.557 | 5.848 | 0.044 | 1.00 | 1.35 |
| ATOM | 473 | C | LEU | 34 | 6.352 | 7.356 | −2.877 | 1.00 | 0.38 |
| ATOM | 474 | O | LEU | 34 | 7.016 | 8.074 | −2.156 | 1.00 | 0.41 |
| ATOM | 475 | N | LYS | 35 | 6.629 | 7.228 | −4.142 | 1.00 | 0.39 |
| ATOM | 476 | HN | LYS | 35 | 6.081 | 6.644 | −4.711 | 1.00 | 0.41 |
| ATOM | 477 | CA | LYS | 35 | 7.779 | 7.971 | −4.724 | 1.00 | 0.47 |
| ATOM | 478 | HA | LYS | 35 | 8.702 | 7.612 | −4.294 | 1.00 | 0.48 |
| ATOM | 479 | CB | LYS | 35 | 7.798 | 7.765 | −6.239 | 1.00 | 0.60 |
| ATOM | 480 | HB1 | LYS | 35 | 7.495 | 8.677 | −6.730 | 1.00 | 1.08 |
| ATOM | 481 | HB2 | LYS | 35 | 7.115 | 6.970 | −6.501 | 1.00 | 0.93 |
| ATOM | 482 | CG | LYS | 35 | 9.211 | 7.393 | −6.688 | 1.00 | 1.34 |
| ATOM | 483 | HG1 | LYS | 35 | 9.485 | 6.440 | −6.262 | 1.00 | 1.84 |
| ATOM | 484 | HG2 | LYS | 35 | 9.905 | 8.151 | −6.355 | 1.00 | 1.85 |
| ATOM | 485 | CD | LYS | 35 | 9.248 | 7.297 | −8.213 | 1.00 | 1.48 |
| ATOM | 486 | HD1 | LYS | 35 | 9.686 | 8.196 | −8.620 | 1.00 | 1.59 |
| ATOM | 487 | HD2 | LYS | 35 | 8.242 | 7.183 | −8.591 | 1.00 | 1.64 |
| ATOM | 488 | CE | LYS | 35 | 10.091 | 6.091 | −8.627 | 1.00 | 2.33 |
| ATOM | 489 | HE1 | LYS | 35 | 11.059 | 6.148 | −8.153 | 1.00 | 2.75 |
| ATOM | 490 | HE2 | LYS | 35 | 10.214 | 6.088 | −9.700 | 1.00 | 2.70 |
| ATOM | 491 | NZ | LYS | 35 | 9.402 | 4.839 | −8.202 | 1.00 | 3.09 |
| ATOM | 492 | HZ1 | LYS | 35 | 9.038 | 4.955 | −7.235 | 1.00 | 3.47 |
| ATOM | 493 | HZ2 | LYS | 35 | 10.076 | 4.047 | −8.227 | 1.00 | 3.33 |
| ATOM | 494 | HZ3 | LYS | 35 | 8.610 | 4.643 | −8.846 | 1.00 | 3.60 |
| ATOM | 495 | C | LYS | 35 | 7.606 | 9.461 | −4.413 | 1.00 | 0.50 |
| ATOM | 496 | O | LYS | 35 | 8.552 | 10.155 | −4.099 | 1.00 | 0.55 |
| ATOM | 497 | N | SER | 36 | 6.399 | 9.952 | −4.493 | 1.00 | 0.54 |
| ATOM | 498 | HN | SER | 36 | 5.652 | 9.369 | −4.743 | 1.00 | 0.52 |
| ATOM | 499 | CA | SER | 36 | 6.148 | 11.392 | −4.196 | 1.00 | 0.66 |
| ATOM | 500 | HA | SER | 36 | 6.722 | 12.002 | −4.877 | 1.00 | 0.73 |
| ATOM | 501 | CB | SER | 36 | 4.661 | 11.704 | −4.365 | 1.00 | 0.75 |
| ATOM | 502 | HB1 | SER | 36 | 4.479 | 12.737 | −4.100 | 1.00 | 0.88 |
| ATOM | 503 | HB2 | SER | 36 | 4.081 | 11.066 | −3.722 | 1.00 | 0.71 |
| ATOM | 504 | OG | SER | 36 | 4.282 | 11.475 | −5.716 | 1.00 | 0.81 |
| ATOM | 505 | HG | SER | 36 | 4.052 | 10.548 | −5.807 | 1.00 | 1.17 |
| ATOM | 506 | C | SER | 36 | 6.570 | 11.704 | −2.758 | 1.00 | 0.65 |
| ATOM | 507 | O | SER | 36 | 7.065 | 12.774 | −2.467 | 1.00 | 0.75 |
| ATOM | 508 | N | GLU | 37 | 6.364 | 10.781 | −1.856 | 1.00 | 0.59 |
| ATOM | 509 | HN | GLU | 37 | 5.953 | 9.929 | −2.115 | 1.00 | 0.55 |
| ATOM | 510 | CA | GLU | 37 | 6.736 | 11.025 | −0.431 | 1.00 | 0.66 |
| ATOM | 511 | HA | GLU | 37 | 6.817 | 12.086 | −0.251 | 1.00 | 0.77 |
| ATOM | 512 | CB | GLU | 37 | 5.658 | 10.433 | 0.482 | 1.00 | 0.72 |
| ATOM | 513 | HB1 | GLU | 37 | 6.004 | 10.451 | 1.504 | 1.00 | 0.80 |
| ATOM | 514 | HB2 | GLU | 37 | 5.458 | 9.413 | 0.187 | 1.00 | 0.65 |
| ATOM | 515 | CG | GLU | 37 | 4.375 | 11.259 | 0.366 | 1.00 | 0.86 |
| ATOM | 516 | HG1 | GLU | 37 | 4.043 | 11.267 | −0.661 | 1.00 | 0.89 |
| ATOM | 517 | HG2 | GLU | 37 | 4.568 | 12.271 | 0.691 | 1.00 | 1.11 |
| ATOM | 518 | CD | GLU | 37 | 3.288 | 10.638 | 1.245 | 1.00 | 1.09 |
| ATOM | 519 | OE1 | GLU | 37 | 3.179 | 9.423 | 1.249 | 1.00 | 1.61 |
| ATOM | 520 | OE2 | GLU | 37 | 2.582 | 11.388 | 1.899 | 1.00 | 1.69 |
| ATOM | 521 | C | GLU | 37 | 8.076 | 10.349 | −0.132 | 1.00 | 0.58 |
| ATOM | 522 | O | GLU | 37 | 8.509 | 10.281 | 1.001 | 1.00 | 0.65 |
| ATOM | 523 | N | TYR | 38 | 8.737 | 9.860 | −1.145 | 1.00 | 0.49 |
| ATOM | 524 | HN | TYR | 38 | 8.369 | 9.936 | −2.049 | 1.00 | 0.47 |
| ATOM | 525 | CA | TYR | 38 | 10.057 | 9.200 | −0.937 | 1.00 | 0.48 |
| ATOM | 526 | HA | TYR | 38 | 10.389 | 8.762 | −1.866 | 1.00 | 0.47 |
| ATOM | 527 | CB | TYR | 38 | 11.075 | 10.242 | −0.473 | 1.00 | 0.62 |
| ATOM | 528 | HB1 | TYR | 38 | 12.041 | 9.775 | −0.354 | 1.00 | 0.65 |
| ATOM | 529 | HB2 | TYR | 38 | 10.758 | 10.661 | 0.469 | 1.00 | 0.69 |
| ATOM | 530 | CG | TYR | 38 | 11.171 | 11.338 | −1.507 | 1.00 | 0.72 |
| ATOM | 531 | CD1 | TYR | 38 | 10.437 | 12.519 | −1.340 | 1.00 | 0.83 |
| ATOM | 532 | HD1 | TYR | 38 | 9.807 | 12.645 | −0.472 | 1.00 | 0.89 |
| ATOM | 533 | CD2 | TYR | 38 | 11.988 | 11.173 | −2.631 | 1.00 | 0.77 |
| ATOM | 534 | HD2 | TYR | 38 | 12.553 | 10.262 | −2.760 | 1.00 | 0.78 |
| ATOM | 535 | CE1 | TYR | 38 | 10.520 | 13.535 | −2.298 | 1.00 | 0.95 |
| ATOM | 536 | HE1 | TYR | 38 | 9.954 | 14.446 | −2.170 | 1.00 | 1.07 |
| ATOM | 537 | CE2 | TYR | 38 | 12.071 | 12.191 | −3.590 | 1.00 | 0.89 |
| ATOM | 538 | HE2 | TYR | 38 | 12.702 | 12.065 | −4.457 | 1.00 | 0.98 |
| ATOM | 539 | CZ | TYR | 38 | 11.338 | 13.372 | −3.423 | 1.00 | 0.96 |
| ATOM | 540 | OH | TYR | 38 | 11.420 | 14.374 | −4.368 | 1.00 | 1.10 |
| ATOM | 541 | HH | TYR | 38 | 10.671 | 14.284 | −4.961 | 1.00 | 1.26 |
| ATOM | 542 | C | TYR | 38 | 9.923 | 8.105 | 0.122 | 1.00 | 0.45 |
| ATOM | 543 | O | TYR | 38 | 10.871 | 7.770 | 0.804 | 1.00 | 0.53 |
| ATOM | 544 | N | SER | 39 | 8.753 | 7.544 | 0.253 | 1.00 | 0.41 |
| ATOM | 545 | HN | SER | 39 | 8.009 | 7.832 | −0.316 | 1.00 | 0.41 |
| ATOM | 546 | CA | SER | 39 | 8.539 | 6.463 | 1.257 | 1.00 | 0.44 |
| ATOM | 547 | HA | SER | 39 | 9.338 | 6.481 | 1.984 | 1.00 | 0.51 |
| ATOM | 548 | CB | SER | 39 | 7.201 | 6.687 | 1.965 | 1.00 | 0.52 |
| ATOM | 549 | HB1 | SER | 39 | 7.369 | 7.244 | 2.877 | 1.00 | 1.14 |
| ATOM | 550 | HB2 | SER | 39 | 6.750 | 5.741 | 2.208 | 1.00 | 1.14 |
| ATOM | 551 | OG | SER | 39 | 6.333 | 7.417 | 1.106 | 1.00 | 1.45 |
| ATOM | 552 | HG | SER | 39 | 5.440 | 7.094 | 1.244 | 1.00 | 1.88 |
| ATOM | 553 | C | SER | 39 | 8.544 | 5.114 | 0.532 | 1.00 | 0.37 |
| ATOM | 554 | O | SER | 39 | 7.896 | 4.168 | 0.936 | 1.00 | 0.37 |
| ATOM | 555 | N | GLU | 40 | 9.276 | 5.026 | −0.546 | 1.00 | 0.36 |
| ATOM | 556 | HN | GLU | 40 | 9.787 | 5.803 | −0.853 | 1.00 | 0.40 |
| ATOM | 557 | CA | GLU | 40 | 9.331 | 3.751 | −1.314 | 1.00 | 0.35 |
| ATOM | 558 | HA | GLU | 40 | 8.330 | 3.447 | −1.581 | 1.00 | 0.35 |
| ATOM | 559 | CB | GLU | 40 | 10.156 | 3.962 | −2.584 | 1.00 | 0.41 |
| ATOM | 560 | HB1 | GLU | 40 | 9.749 | 4.789 | −3.146 | 1.00 | 0.45 |
| ATOM | 561 | HB2 | GLU | 40 | 10.122 | 3.065 | −3.185 | 1.00 | 0.42 |
| ATOM | 562 | CG | GLU | 40 | 11.606 | 4.270 | −2.204 | 1.00 | 0.46 |
| ATOM | 563 | HG1 | GLU | 40 | 12.083 | 3.370 | −1.847 | 1.00 | 0.91 |
| ATOM | 564 | HG2 | GLU | 40 | 11.622 | 5.020 | −1.426 | 1.00 | 0.86 |
| ATOM | 565 | CD | GLU | 40 | 12.356 | 4.789 | −3.431 | 1.00 | 1.09 |
| ATOM | 566 | OE1 | GLU | 40 | 11.780 | 4.769 | −4.506 | 1.00 | 1.76 |
| ATOM | 567 | OE2 | GLU | 40 | 13.496 | 5.196 | −3.276 | 1.00 | 1.77 |
| ATOM | 568 | C | GLU | 40 | 9.982 | 2.668 | −0.455 | 1.00 | 0.37 |
| ATOM | 569 | O | GLU | 40 | 9.820 | 1.494 | −0.694 | 1.00 | 0.37 |
| ATOM | 570 | N | GLU | 41 | 10.709 | 3.045 | 0.551 | 1.00 | 0.43 |
| ATOM | 571 | HN | GLU | 41 | 10.830 | 3.999 | 0.745 | 1.00 | 0.47 |
| ATOM | 572 | CA | GLU | 41 | 11.341 | 2.018 | 1.420 | 1.00 | 0.50 |
| ATOM | 573 | HA | GLU | 41 | 11.954 | 1.361 | 0.822 | 1.00 | 0.54 |
| ATOM | 574 | CB | GLU | 41 | 12.203 | 2.712 | 2.476 | 1.00 | 0.63 |
| ATOM | 575 | HB1 | GLU | 41 | 12.956 | 3.314 | 1.989 | 1.00 | 0.75 |
| ATOM | 576 | HB2 | GLU | 41 | 12.682 | 1.968 | 3.096 | 1.00 | 0.65 |
| ATOM | 577 | CG | GLU | 41 | 11.320 | 3.611 | 3.346 | 1.00 | 0.65 |
| ATOM | 578 | HG1 | GLU | 41 | 10.948 | 3.045 | 4.186 | 1.00 | 0.98 |
| ATOM | 579 | HG2 | GLU | 41 | 10.489 | 3.975 | 2.760 | 1.00 | 0.79 |
| ATOM | 580 | CD | GLU | 41 | 12.143 | 4.794 | 3.857 | 1.00 | 1.10 |
| ATOM | 581 | OE1 | GLU | 41 | 11.545 | 5.789 | 4.231 | 1.00 | 1.72 |
| ATOM | 582 | OE2 | GLU | 41 | 13.358 | 4.687 | 3.862 | 1.00 | 1.73 |
| ATOM | 583 | C | GLU | 41 | 10.232 | 1.212 | 2.104 | 1.00 | 0.45 |
| ATOM | 584 | O | GLU | 41 | 10.379 | 0.040 | 2.387 | 1.00 | 0.45 |
| ATOM | 585 | N | ASN | 42 | 9.124 | 1.849 | 2.378 | 1.00 | 0.42 |
| ATOM | 586 | HN | ASN | 42 | 9.040 | 2.797 | 2.143 | 1.00 | 0.44 |
| ATOM | 587 | CA | ASN | 42 | 7.990 | 1.153 | 3.053 | 1.00 | 0.41 |
| ATOM | 588 | HA | ASN | 42 | 8.316 | 0.797 | 4.018 | 1.00 | 0.45 |
| ATOM | 589 | CB | ASN | 42 | 6.842 | 2.144 | 3.250 | 1.00 | 0.44 |
| ATOM | 590 | HB1 | ASN | 42 | 5.959 | 1.613 | 3.574 | 1.00 | 0.46 |
| ATOM | 591 | HB2 | ASN | 42 | 6.638 | 2.646 | 2.316 | 1.00 | 0.41 |
| ATOM | 592 | CG | ASN | 42 | 7.231 | 3.176 | 4.308 | 1.00 | 0.51 |
| ATOM | 593 | OD1 | ASN | 42 | 8.056 | 2.910 | 5.159 | 1.00 | 1.00 |
| ATOM | 594 | ND2 | ASN | 42 | 6.667 | 4.352 | 4.291 | 1.00 | 1.28 |
| ATOM | 595 | HD21 | ASN | 42 | 6.002 | 4.566 | 3.604 | 1.00 | 2.00 |
| ATOM | 596 | HD22 | ASN | 42 | 6.907 | 5.020 | 4.965 | 1.00 | 1.32 |
| ATOM | 597 | C | ASN | 42 | 7.485 | −0.035 | 2.223 | 1.00 | 0.36 |
| ATOM | 598 | O | ASN | 42 | 7.276 | −1.113 | 2.744 | 1.00 | 0.36 |
| ATOM | 599 | N | ILE | 43 | 7.260 | 0.145 | 0.946 | 1.00 | 0.36 |
| ATOM | 600 | HN | ILE | 43 | 7.413 | 1.021 | 0.534 | 1.00 | 0.38 |
| ATOM | 601 | CA | ILE | 43 | 6.741 | −0.993 | 0.129 | 1.00 | 0.35 |
| ATOM | 602 | HA | ILE | 43 | 5.915 | −1.439 | 0.665 | 1.00 | 0.36 |
| ATOM | 603 | CB | ILE | 43 | 6.224 | −0.485 | −1.234 | 1.00 | 0.36 |
| ATOM | 604 | HB | ILE | 43 | 5.556 | 0.347 | −1.071 | 1.00 | 0.40 |
| ATOM | 605 | CG1 | ILE | 43 | 5.472 | −1.611 | −1.958 | 1.00 | 0.46 |
| ATOM | 606 | HG11 | ILE | 43 | 6.092 | −2.494 | −1.985 | 1.00 | 0.55 |
| ATOM | 607 | HG12 | ILE | 43 | 5.255 | −1.297 | −2.969 | 1.00 | 0.49 |
| ATOM | 608 | CG2 | ILE | 43 | 7.383 | −0.019 | −2.115 | 1.00 | 0.45 |
| ATOM | 609 | HG21 | ILE | 43 | 7.549 | −0.739 | −2.902 | 1.00 | 1.14 |
| ATOM | 610 | HG22 | ILE | 43 | 8.269 | 0.075 | −1.524 | 1.00 | 1.12 |
| ATOM | 611 | HG23 | ILE | 43 | 7.139 | 0.938 | −2.552 | 1.00 | 1.10 |
| ATOM | 612 | CD1 | ILE | 43 | 4.154 | −1.937 | −1.238 | 1.00 | 0.59 |
| ATOM | 613 | HD11 | ILE | 43 | 4.247 | −2.890 | −0.737 | 1.00 | 1.19 |
| ATOM | 614 | HD12 | ILE | 43 | 3.354 | −1.991 | −1.962 | 1.00 | 1.22 |
| ATOM | 615 | HD13 | ILE | 43 | 3.928 | −1.171 | −0.513 | 1.00 | 1.18 |
| ATOM | 616 | C | ILE | 43 | 7.829 | −2.063 | −0.035 | 1.00 | 0.36 |
| ATOM | 617 | O | ILE | 43 | 7.550 | −3.244 | 0.022 | 1.00 | 0.36 |
| ATOM | 618 | N | ASP | 44 | 9.067 | −1.678 | −0.210 | 1.00 | 0.38 |
| ATOM | 619 | HN | ASP | 44 | 9.291 | −0.724 | −0.235 | 1.00 | 0.39 |
| ATOM | 620 | CA | ASP | 44 | 10.137 | −2.707 | −0.339 | 1.00 | 0.42 |
| ATOM | 621 | HA | ASP | 44 | 9.903 | −3.382 | −1.149 | 1.00 | 0.44 |
| ATOM | 622 | CB | ASP | 44 | 11.481 | −2.023 | −0.601 | 1.00 | 0.48 |
| ATOM | 623 | HB1 | ASP | 44 | 12.277 | −2.749 | −0.521 | 1.00 | 0.52 |
| ATOM | 624 | HB2 | ASP | 44 | 11.635 | −1.239 | 0.126 | 1.00 | 0.48 |
| ATOM | 625 | CG | ASP | 44 | 11.483 | −1.423 | −2.008 | 1.00 | 0.53 |
| ATOM | 626 | OD1 | ASP | 44 | 10.684 | −1.862 | −2.819 | 1.00 | 0.98 |
| ATOM | 627 | OD2 | ASP | 44 | 12.282 | −0.533 | −2.250 | 1.00 | 0.85 |
| ATOM | 628 | C | ASP | 44 | 10.205 | −3.480 | 0.978 | 1.00 | 0.38 |
| ATOM | 629 | O | ASP | 44 | 10.286 | −4.692 | 1.005 | 1.00 | 0.40 |
| ATOM | 630 | N | PHE | 45 | 10.144 | −2.771 | 2.072 | 1.00 | 0.36 |
| ATOM | 631 | HN | PHE | 45 | 10.059 | −1.797 | 2.012 | 1.00 | 0.37 |
| ATOM | 632 | CA | PHE | 45 | 10.172 | −3.426 | 3.408 | 1.00 | 0.34 |
| ATOM | 633 | HA | PHE | 45 | 11.069 | −4.019 | 3.505 | 1.00 | 0.36 |
| ATOM | 634 | CB | PHE | 45 | 10.154 | −2.333 | 4.482 | 1.00 | 0.35 |
| ATOM | 635 | HB1 | PHE | 45 | 9.259 | −1.739 | 4.371 | 1.00 | 0.36 |
| ATOM | 636 | HB2 | PHE | 45 | 11.019 | −1.699 | 4.358 | 1.00 | 0.40 |
| ATOM | 637 | CG | PHE | 45 | 10.180 | −2.942 | 5.864 | 1.00 | 0.34 |
| ATOM | 638 | CD1 | PHE | 45 | 8.979 | −3.176 | 6.545 | 1.00 | 0.35 |
| ATOM | 639 | HD1 | PHE | 45 | 8.037 | −2.931 | 6.077 | 1.00 | 0.38 |
| ATOM | 640 | CD2 | PHE | 45 | 11.402 | −3.260 | 6.469 | 1.00 | 0.38 |
| ATOM | 641 | HD2 | PHE | 45 | 12.328 | −3.080 | 5.944 | 1.00 | 0.43 |
| ATOM | 642 | CE1 | PHE | 45 | 8.998 | −3.727 | 7.832 | 1.00 | 0.39 |
| ATOM | 643 | HE1 | PHE | 45 | 8.070 | −3.904 | 8.358 | 1.00 | 0.44 |
| ATOM | 644 | CE2 | PHE | 45 | 11.422 | −3.814 | 7.756 | 1.00 | 0.41 |
| ATOM | 645 | HE2 | PHE | 45 | 12.364 | −4.060 | 8.223 | 1.00 | 0.47 |
| ATOM | 646 | CZ | PHE | 45 | 10.220 | −4.047 | 8.437 | 1.00 | 0.40 |
| ATOM | 647 | HZ | PHE | 45 | 10.236 | −4.472 | 9.430 | 1.00 | 0.45 |
| ATOM | 648 | C | PHE | 45 | 8.942 | −4.325 | 3.546 | 1.00 | 0.32 |
| ATOM | 649 | O | PHE | 45 | 9.010 | −5.420 | 4.068 | 1.00 | 0.33 |
| ATOM | 650 | N | TRP | 46 | 7.816 | −3.863 | 3.074 | 1.00 | 0.32 |
| ATOM | 651 | HN | TRP | 46 | 7.791 | −2.977 | 2.656 | 1.00 | 0.33 |
| ATOM | 652 | CA | TRP | 46 | 6.569 | −4.673 | 3.164 | 1.00 | 0.33 |
| ATOM | 653 | HA | TRP | 46 | 6.353 | −4.885 | 4.201 | 1.00 | 0.34 |
| ATOM | 654 | CB | TRP | 46 | 5.409 | −3.882 | 2.553 | 1.00 | 0.36 |
| ATOM | 655 | HB1 | TRP | 46 | 5.625 | −3.672 | 1.516 | 1.00 | 0.36 |
| ATOM | 656 | HB2 | TRP | 46 | 5.287 | −2.953 | 3.089 | 1.00 | 0.38 |
| ATOM | 657 | CG | TRP | 46 | 4.147 | −4.679 | 2.647 | 1.00 | 0.38 |
| ATOM | 658 | CD1 | TRP | 46 | 3.381 | −4.785 | 3.758 | 1.00 | 0.47 |
| ATOM | 659 | HD1 | TRP | 46 | 3.586 | −4.316 | 4.709 | 1.00 | 0.55 |
| ATOM | 660 | CD2 | TRP | 46 | 3.487 | −5.471 | 1.618 | 1.00 | 0.42 |
| ATOM | 661 | NE1 | TRP | 46 | 2.295 | −5.593 | 3.477 | 1.00 | 0.50 |
| ATOM | 662 | HE1 | TRP | 46 | 1.587 | −5.830 | 4.112 | 1.00 | 0.58 |
| ATOM | 663 | CE2 | TRP | 46 | 2.315 | −6.041 | 2.171 | 1.00 | 0.47 |
| ATOM | 664 | CE3 | TRP | 46 | 3.787 | −5.750 | 0.273 | 1.00 | 0.50 |
| ATOM | 665 | HE3 | TRP | 46 | 4.674 | −5.331 | −0.177 | 1.00 | 0.52 |
| ATOM | 666 | CZ2 | TRP | 46 | 1.471 | −6.856 | 1.417 | 1.00 | 0.54 |
| ATOM | 667 | HZ2 | TRP | 46 | 0.582 | −7.278 | 1.863 | 1.00 | 0.58 |
| ATOM | 668 | CZ3 | TRP | 46 | 2.939 | −6.571 | −0.490 | 1.00 | 0.61 |
| ATOM | 669 | HZ3 | TRP | 46 | 3.179 | −6.779 | −1.522 | 1.00 | 0.73 |
| ATOM | 670 | CH2 | TRP | 46 | 1.784 | −7.123 | 0.082 | 1.00 | 0.61 |
| ATOM | 671 | HH2 | TRP | 46 | 1.136 | −7.753 | −0.509 | 1.00 | 0.70 |
| ATOM | 672 | C | TRP | 46 | 6.758 | −5.991 | 2.403 | 1.00 | 0.34 |
| ATOM | 673 | O | TRP | 46 | 6.316 | −7.037 | 2.836 | 1.00 | 0.36 |
| ATOM | 674 | N | ILE | 47 | 7.406 | −5.948 | 1.266 | 1.00 | 0.35 |
| ATOM | 675 | HN | ILE | 47 | 7.750 | −5.094 | 0.930 | 1.00 | 0.35 |
| ATOM | 676 | CA | ILE | 47 | 7.613 | −7.199 | 0.476 | 1.00 | 0.39 |
| ATOM | 677 | HA | ILE | 47 | 6.659 | −7.670 | 0.292 | 1.00 | 0.41 |
| ATOM | 678 | CB | ILE | 47 | 8.280 | −6.857 | −0.859 | 1.00 | 0.44 |
| ATOM | 679 | HB | ILE | 47 | 9.177 | −6.286 | −0.669 | 1.00 | 0.44 |
| ATOM | 680 | CG1 | ILE | 47 | 7.308 | −6.033 | −1.715 | 1.00 | 0.46 |
| ATOM | 681 | HG11 | ILE | 47 | 6.922 | −5.214 | −1.127 | 1.00 | 0.43 |
| ATOM | 682 | HG12 | ILE | 47 | 6.488 | −6.663 | −2.030 | 1.00 | 0.48 |
| ATOM | 683 | CG2 | ILE | 47 | 8.643 | −8.149 | −1.595 | 1.00 | 0.50 |
| ATOM | 684 | HG21 | ILE | 47 | 9.497 | −8.607 | −1.119 | 1.00 | 1.26 |
| ATOM | 685 | HG22 | ILE | 47 | 8.882 | −7.924 | −2.623 | 1.00 | 1.09 |
| ATOM | 686 | HG23 | ILE | 47 | 7.805 | −8.830 | −1.561 | 1.00 | 1.06 |
| ATOM | 687 | CD1 | ILE | 47 | 8.023 | −5.475 | −2.954 | 1.00 | 0.53 |
| ATOM | 688 | HD11 | ILE | 47 | 7.886 | −6.152 | −3.783 | 1.00 | 1.12 |
| ATOM | 689 | HD12 | ILE | 47 | 9.077 | −5.362 | −2.752 | 1.00 | 1.14 |
| ATOM | 690 | HD13 | ILE | 47 | 7.602 | −4.513 | −3.206 | 1.00 | 1.18 |
| ATOM | 691 | C | ILE | 47 | 8.512 | −8.157 | 1.260 | 1.00 | 0.38 |
| ATOM | 692 | O | ILE | 47 | 8.271 | −9.347 | 1.309 | 1.00 | 0.39 |
| ATOM | 693 | N | SER | 48 | 9.538 | −7.649 | 1.884 | 1.00 | 0.38 |
| ATOM | 694 | HN | SER | 48 | 9.713 | −6.686 | 1.842 | 1.00 | 0.38 |
| ATOM | 695 | CA | SER | 48 | 10.435 | −8.537 | 2.672 | 1.00 | 0.41 |
| ATOM | 696 | HA | SER | 48 | 10.745 | −9.373 | 2.060 | 1.00 | 0.43 |
| ATOM | 697 | CB | SER | 48 | 11.666 | −7.753 | 3.131 | 1.00 | 0.43 |
| ATOM | 698 | HB1 | SER | 48 | 12.173 | −7.343 | 2.267 | 1.00 | 0.45 |
| ATOM | 699 | HB2 | SER | 48 | 12.337 | −8.409 | 3.659 | 1.00 | 0.46 |
| ATOM | 700 | OG | SER | 48 | 11.257 | −6.703 | 3.998 | 1.00 | 0.43 |
| ATOM | 701 | HG | SER | 48 | 11.122 | −7.077 | 4.872 | 1.00 | 0.98 |
| ATOM | 702 | C | SER | 48 | 9.666 | −9.051 | 3.888 | 1.00 | 0.41 |
| ATOM | 703 | O | SER | 48 | 9.778 | −10.198 | 4.272 | 1.00 | 0.42 |
| ATOM | 704 | N | CYS | 49 | 8.879 | −8.204 | 4.493 | 1.00 | 0.41 |
| ATOM | 705 | HN | CYS | 49 | 8.803 | −7.285 | 4.160 | 1.00 | 0.41 |
| ATOM | 706 | CA | CYS | 49 | 8.088 | −8.628 | 5.681 | 1.00 | 0.44 |
| ATOM | 707 | HA | CYS | 49 | 8.752 | −9.033 | 6.431 | 1.00 | 0.45 |
| ATOM | 708 | CB | CYS | 49 | 7.347 | −7.420 | 6.253 | 1.00 | 0.49 |
| ATOM | 709 | HB1 | CYS | 49 | 6.369 | −7.726 | 6.595 | 1.00 | 1.07 |
| ATOM | 710 | HB2 | CYS | 49 | 7.241 | −6.666 | 5.487 | 1.00 | 1.09 |
| ATOM | 711 | SG | CYS | 49 | 8.285 | −6.738 | 7.642 | 1.00 | 1.57 |
| ATOM | 712 | HG | CYS | 49 | 7.704 | −6.155 | 8.136 | 1.00 | 1.99 |
| ATOM | 713 | C | CYS | 49 | 7.073 | −9.695 | 5.262 | 1.00 | 0.44 |
| ATOM | 714 | O | CYS | 49 | 6.812 | −10.635 | 5.987 | 1.00 | 0.45 |
| ATOM | 715 | N | GLU | 50 | 6.497 | −9.556 | 4.097 | 1.00 | 0.46 |
| ATOM | 716 | HN | GLU | 50 | 6.722 | −8.791 | 3.529 | 1.00 | 0.46 |
| ATOM | 717 | CA | GLU | 50 | 5.498 | −10.563 | 3.636 | 1.00 | 0.49 |
| ATOM | 718 | HA | GLU | 50 | 4.673 | −10.598 | 4.332 | 1.00 | 0.52 |
| ATOM | 719 | CB | GLU | 50 | 4.980 | −10.171 | 2.250 | 1.00 | 0.52 |
| ATOM | 720 | HB1 | GLU | 50 | 4.419 | −10.991 | 1.829 | 1.00 | 0.93 |
| ATOM | 721 | HB2 | GLU | 50 | 5.817 | −9.938 | 1.607 | 1.00 | 0.86 |
| ATOM | 722 | CG | GLU | 50 | 4.074 | −8.945 | 2.371 | 1.00 | 0.83 |
| ATOM | 723 | HG1 | GLU | 50 | 4.303 | −8.247 | 1.580 | 1.00 | 1.28 |
| ATOM | 724 | HG2 | GLU | 50 | 4.236 | −8.471 | 3.329 | 1.00 | 1.55 |
| ATOM | 725 | CD | GLU | 50 | 2.611 | −9.379 | 2.257 | 1.00 | 1.44 |
| ATOM | 726 | OE1 | GLU | 50 | 1.829 | −8.989 | 3.108 | 1.00 | 2.08 |
| ATOM | 727 | OE2 | GLU | 50 | 2.298 | −10.094 | 1.319 | 1.00 | 2.13 |
| ATOM | 728 | C | GLU | 50 | 6.162 | −11.937 | 3.561 | 1.00 | 0.46 |
| ATOM | 729 | O | GLU | 50 | 5.635 | −12.918 | 4.047 | 1.00 | 0.48 |
| ATOM | 730 | N | GLU | 51 | 7.325 | −12.014 | 2.976 | 1.00 | 0.43 |
| ATOM | 731 | HN | GLU | 51 | 7.744 | −11.209 | 2.604 | 1.00 | 0.43 |
| ATOM | 732 | CA | GLU | 51 | 8.025 | −13.325 | 2.899 | 1.00 | 0.43 |
| ATOM | 733 | HA | GLU | 51 | 7.408 | −14.035 | 2.367 | 1.00 | 0.47 |
| ATOM | 734 | CB | GLU | 51 | 9.363 | −13.157 | 2.175 | 1.00 | 0.44 |
| ATOM | 735 | HB1 | GLU | 51 | 9.941 | −14.064 | 2.274 | 1.00 | 0.46 |
| ATOM | 736 | HB2 | GLU | 51 | 9.907 | −12.333 | 2.612 | 1.00 | 0.42 |
| ATOM | 737 | CG | GLU | 51 | 9.114 | −12.874 | 0.692 | 1.00 | 0.51 |
| ATOM | 738 | HG1 | GLU | 51 | 8.465 | −12.018 | 0.592 | 1.00 | 0.73 |
| ATOM | 739 | HG2 | GLU | 51 | 8.649 | −13.736 | 0.235 | 1.00 | 0.69 |
| ATOM | 740 | CD | GLU | 51 | 10.446 | −12.585 | −0.002 | 1.00 | 0.63 |
| ATOM | 741 | OE1 | GLU | 51 | 10.431 | −12.357 | −1.200 | 1.00 | 1.41 |
| ATOM | 742 | OE2 | GLU | 51 | 11.460 | −12.595 | 0.678 | 1.00 | 1.24 |
| ATOM | 743 | C | GLU | 51 | 8.272 | −13.822 | 4.321 | 1.00 | 0.40 |
| ATOM | 744 | O | GLU | 51 | 8.141 | −14.992 | 4.619 | 1.00 | 0.41 |
| ATOM | 745 | N | TYR | 52 | 8.633 | −12.928 | 5.197 | 1.00 | 0.37 |
| ATOM | 746 | HN | TYR | 52 | 8.731 | −11.992 | 4.924 | 1.00 | 0.37 |
| ATOM | 747 | CA | TYR | 52 | 8.899 | −13.316 | 6.607 | 1.00 | 0.35 |
| ATOM | 748 | HA | TYR | 52 | 9.696 | −14.043 | 6.639 | 1.00 | 0.34 |
| ATOM | 749 | CB | TYR | 52 | 9.312 | −12.067 | 7.390 | 1.00 | 0.36 |
| ATOM | 750 | HB1 | TYR | 52 | 8.442 | −11.454 | 7.573 | 1.00 | 0.40 |
| ATOM | 751 | HB2 | TYR | 52 | 10.032 | −11.505 | 6.814 | 1.00 | 0.38 |
| ATOM | 752 | CG | TYR | 52 | 9.929 | −12.468 | 8.708 | 1.00 | 0.31 |
| ATOM | 753 | CD1 | TYR | 52 | 11.322 | −12.552 | 8.828 | 1.00 | 0.30 |
| ATOM | 754 | HD1 | TYR | 52 | 11.951 | −12.330 | 7.979 | 1.00 | 0.33 |
| ATOM | 755 | CD2 | TYR | 52 | 9.113 | −12.755 | 9.807 | 1.00 | 0.30 |
| ATOM | 756 | HD2 | TYR | 52 | 8.039 | −12.690 | 9.715 | 1.00 | 0.33 |
| ATOM | 757 | CE1 | TYR | 52 | 11.899 | −12.921 | 10.048 | 1.00 | 0.28 |
| ATOM | 758 | HE1 | TYR | 52 | 12.973 | −12.986 | 10.139 | 1.00 | 0.29 |
| ATOM | 759 | CE2 | TYR | 52 | 9.690 | −13.125 | 11.027 | 1.00 | 0.29 |
| ATOM | 760 | HE2 | TYR | 52 | 9.061 | −13.347 | 11.875 | 1.00 | 0.31 |
| ATOM | 761 | CZ | TYR | 52 | 11.083 | −13.209 | 11.148 | 1.00 | 0.28 |
| ATOM | 762 | OH | TYR | 52 | 11.652 | −13.575 | 12.351 | 1.00 | 0.29 |
| ATOM | 763 | HH | TYR | 52 | 11.898 | −12.774 | 12.819 | 1.00 | 0.88 |
| ATOM | 764 | C | TYR | 52 | 7.627 | −13.919 | 7.220 | 1.00 | 0.38 |
| ATOM | 765 | O | TYR | 52 | 7.678 | −14.896 | 7.941 | 1.00 | 0.38 |
| ATOM | 766 | N | LYS | 53 | 6.485 | −13.343 | 6.940 | 1.00 | 0.43 |
| ATOM | 767 | HN | LYS | 53 | 6.464 | −12.555 | 6.358 | 1.00 | 0.44 |
| ATOM | 768 | CA | LYS | 53 | 5.212 | −13.884 | 7.508 | 1.00 | 0.48 |
| ATOM | 769 | HA | LYS | 53 | 5.331 | −14.019 | 8.573 | 1.00 | 0.49 |
| ATOM | 770 | CB | LYS | 53 | 4.060 | −12.909 | 7.256 | 1.00 | 0.58 |
| ATOM | 771 | HB1 | LYS | 53 | 3.121 | −13.421 | 7.407 | 1.00 | 0.64 |
| ATOM | 772 | HB2 | LYS | 53 | 4.112 | −12.549 | 6.239 | 1.00 | 0.58 |
| ATOM | 773 | CG | LYS | 53 | 4.152 | −11.724 | 8.219 | 1.00 | 0.66 |
| ATOM | 774 | HG1 | LYS | 53 | 5.045 | −11.156 | 8.008 | 1.00 | 0.79 |
| ATOM | 775 | HG2 | LYS | 53 | 4.187 | −12.088 | 9.236 | 1.00 | 0.94 |
| ATOM | 776 | CD | LYS | 53 | 2.923 | −10.826 | 8.033 | 1.00 | 0.81 |
| ATOM | 777 | HD1 | LYS | 53 | 2.035 | −11.438 | 7.968 | 1.00 | 1.25 |
| ATOM | 778 | HD2 | LYS | 53 | 3.032 | −10.255 | 7.122 | 1.00 | 1.23 |
| ATOM | 779 | CE | LYS | 53 | 2.791 | −9.870 | 9.220 | 1.00 | 1.12 |
| ATOM | 780 | HE1 | LYS | 53 | 2.836 | −10.430 | 10.142 | 1.00 | 1.54 |
| ATOM | 781 | HE2 | LYS | 53 | 1.844 | −9.356 | 9.164 | 1.00 | 1.59 |
| ATOM | 782 | NZ | LYS | 53 | 3.902 | −8.878 | 9.186 | 1.00 | 2.03 |
| ATOM | 783 | HZ1 | LYS | 53 | 4.149 | −8.668 | 8.198 | 1.00 | 2.49 |
| ATOM | 784 | HZ2 | LYS | 53 | 4.733 | −9.271 | 9.675 | 1.00 | 2.51 |
| ATOM | 785 | HZ3 | LYS | 53 | 3.602 | −8.003 | 9.659 | 1.00 | 2.52 |
| ATOM | 786 | C | LYS | 53 | 4.859 | −15.231 | 6.872 | 1.00 | 0.48 |
| ATOM | 787 | O | LYS | 53 | 4.084 | −15.991 | 7.417 | 1.00 | 0.52 |
| ATOM | 788 | N | LYS | 54 | 5.394 | −15.537 | 5.722 | 1.00 | 0.46 |
| ATOM | 789 | HN | LYS | 54 | 6.008 | −14.915 | 5.278 | 1.00 | 0.46 |
| ATOM | 790 | CA | LYS | 54 | 5.041 | −16.836 | 5.083 | 1.00 | 0.50 |
| ATOM | 791 | HA | LYS | 54 | 4.020 | −17.089 | 5.331 | 1.00 | 0.53 |
| ATOM | 792 | CB | LYS | 54 | 5.178 | −16.721 | 3.562 | 1.00 | 0.56 |
| ATOM | 793 | HB1 | LYS | 54 | 5.090 | −17.700 | 3.117 | 1.00 | 0.60 |
| ATOM | 794 | HB2 | LYS | 54 | 6.142 | −16.298 | 3.318 | 1.00 | 0.54 |
| ATOM | 795 | CG | LYS | 54 | 4.068 | −15.816 | 3.021 | 1.00 | 0.62 |
| ATOM | 796 | HG1 | LYS | 54 | 4.154 | −14.836 | 3.466 | 1.00 | 0.60 |
| ATOM | 797 | HG2 | LYS | 54 | 3.105 | −16.240 | 3.269 | 1.00 | 0.66 |
| ATOM | 798 | CD | LYS | 54 | 4.197 | −15.695 | 1.502 | 1.00 | 0.71 |
| ATOM | 799 | HD1 | LYS | 54 | 4.109 | −16.674 | 1.054 | 1.00 | 0.84 |
| ATOM | 800 | HD2 | LYS | 54 | 5.160 | −15.271 | 1.254 | 1.00 | 0.88 |
| ATOM | 801 | CE | LYS | 54 | 3.086 | −14.790 | 0.965 | 1.00 | 0.89 |
| ATOM | 802 | HE1 | LYS | 54 | 2.472 | −14.449 | 1.785 | 1.00 | 1.34 |
| ATOM | 803 | HE2 | LYS | 54 | 2.478 | −15.343 | 0.265 | 1.00 | 1.45 |
| ATOM | 804 | NZ | LYS | 54 | 3.693 | −13.617 | 0.277 | 1.00 | 1.70 |
| ATOM | 805 | HZ1 | LYS | 54 | 4.116 | −13.922 | −0.622 | 1.00 | 2.20 |
| ATOM | 806 | HZ2 | LYS | 54 | 4.429 | −13.202 | 0.885 | 1.00 | 2.24 |
| ATOM | 807 | HZ3 | LYS | 54 | 2.958 | −12.906 | 0.088 | 1.00 | 2.18 |
| ATOM | 808 | C | LYS | 54 | 5.970 | −17.929 | 5.606 | 1.00 | 0.47 |
| ATOM | 809 | O | LYS | 54 | 5.586 | −19.077 | 5.721 | 1.00 | 0.52 |
| ATOM | 810 | N | ILE | 55 | 7.178 | −17.586 | 5.954 | 1.00 | 0.43 |
| ATOM | 811 | HN | ILE | 55 | 7.468 | −16.654 | 5.877 | 1.00 | 0.43 |
| ATOM | 812 | CA | ILE | 55 | 8.102 | −18.616 | 6.501 | 1.00 | 0.44 |
| ATOM | 813 | HA | ILE | 55 | 8.062 | −19.504 | 5.886 | 1.00 | 0.49 |
| ATOM | 814 | CB | ILE | 55 | 9.531 | −18.067 | 6.523 | 1.00 | 0.42 |
| ATOM | 815 | HB | ILE | 55 | 9.545 | −17.129 | 7.057 | 1.00 | 0.40 |
| ATOM | 816 | CG1 | ILE | 55 | 10.012 | −17.858 | 5.077 | 1.00 | 0.43 |
| ATOM | 817 | HG11 | ILE | 55 | 9.228 | −17.386 | 4.505 | 1.00 | 0.44 |
| ATOM | 818 | HG12 | ILE | 55 | 10.244 | −18.818 | 4.637 | 1.00 | 0.47 |
| ATOM | 819 | CG2 | ILE | 55 | 10.446 | −19.072 | 7.229 | 1.00 | 0.44 |
| ATOM | 820 | HG21 | ILE | 55 | 11.472 | −18.778 | 7.096 | 1.00 | 1.12 |
| ATOM | 821 | HG22 | ILE | 55 | 10.297 | −20.055 | 6.805 | 1.00 | 1.10 |
| ATOM | 822 | HG23 | ILE | 55 | 10.214 | −19.096 | 8.283 | 1.00 | 1.11 |
| ATOM | 823 | CD1 | ILE | 55 | 11.267 | −16.972 | 5.041 | 1.00 | 0.43 |
| ATOM | 824 | HD11 | ILE | 55 | 11.181 | −16.182 | 5.772 | 1.00 | 1.04 |
| ATOM | 825 | HD12 | ILE | 55 | 11.368 | −16.536 | 4.058 | 1.00 | 1.11 |
| ATOM | 826 | HD13 | ILE | 55 | 12.141 | −17.568 | 5.256 | 1.00 | 1.07 |
| ATOM | 827 | C | ILE | 55 | 7.654 | −18.956 | 7.925 | 1.00 | 0.46 |
| ATOM | 828 | O | ILE | 55 | 7.679 | −18.117 | 8.803 | 1.00 | 0.45 |
| ATOM | 829 | N | LYS | 56 | 7.242 | −20.173 | 8.159 | 1.00 | 0.53 |
| ATOM | 830 | HN | LYS | 56 | 7.228 | −20.831 | 7.433 | 1.00 | 0.57 |
| ATOM | 831 | CA | LYS | 56 | 6.786 | −20.564 | 9.526 | 1.00 | 0.60 |
| ATOM | 832 | HA | LYS | 56 | 6.530 | −19.676 | 10.084 | 1.00 | 0.60 |
| ATOM | 833 | CB | LYS | 56 | 5.549 | −21.450 | 9.410 | 1.00 | 0.71 |
| ATOM | 834 | HB1 | LYS | 56 | 5.320 | −21.883 | 10.372 | 1.00 | 0.76 |
| ATOM | 835 | HB2 | LYS | 56 | 5.739 | −22.235 | 8.693 | 1.00 | 0.72 |
| ATOM | 836 | CG | LYS | 56 | 4.372 | −20.592 | 8.941 | 1.00 | 0.76 |
| ATOM | 837 | HG1 | LYS | 56 | 4.605 | −20.155 | 7.982 | 1.00 | 0.92 |
| ATOM | 838 | HG2 | LYS | 56 | 4.196 | −19.804 | 9.660 | 1.00 | 1.08 |
| ATOM | 839 | CD | LYS | 56 | 3.113 | −21.450 | 8.815 | 1.00 | 1.01 |
| ATOM | 840 | HD1 | LYS | 56 | 2.896 | −21.918 | 9.763 | 1.00 | 1.45 |
| ATOM | 841 | HD2 | LYS | 56 | 3.269 | −22.210 | 8.062 | 1.00 | 1.34 |
| ATOM | 842 | CE | LYS | 56 | 1.938 | −20.558 | 8.407 | 1.00 | 1.20 |
| ATOM | 843 | HE1 | LYS | 56 | 2.278 | −19.819 | 7.696 | 1.00 | 1.51 |
| ATOM | 844 | HE2 | LYS | 56 | 1.546 | −20.060 | 9.282 | 1.00 | 1.81 |
| ATOM | 845 | NZ | LYS | 56 | 0.867 | −21.388 | 7.789 | 1.00 | 1.88 |
| ATOM | 846 | HZ1 | LYS | 56 | 0.749 | −21.115 | 6.793 | 1.00 | 2.37 |
| ATOM | 847 | HZ2 | LYS | 56 | −0.027 | −21.236 | 8.299 | 1.00 | 2.42 |
| ATOM | 848 | HZ3 | LYS | 56 | 1.131 | −22.392 | 7.842 | 1.00 | 2.24 |
| ATOM | 849 | C | LYS | 56 | 7.899 | −21.308 | 10.259 | 1.00 | 0.59 |
| ATOM | 850 | O | LYS | 56 | 7.723 | −21.765 | 11.369 | 1.00 | 0.66 |
| ATOM | 851 | N | SER | 57 | 9.044 | −21.421 | 9.651 | 1.00 | 0.54 |
| ATOM | 852 | HN | SER | 57 | 9.159 | −21.038 | 8.757 | 1.00 | 0.52 |
| ATOM | 853 | CA | SER | 57 | 10.182 | −22.120 | 10.314 | 1.00 | 0.56 |
| ATOM | 854 | HA | SER | 57 | 9.802 | −22.839 | 11.018 | 1.00 | 0.62 |
| ATOM | 855 | CB | SER | 57 | 11.010 | −22.840 | 9.251 | 1.00 | 0.60 |
| ATOM | 856 | HB1 | SER | 57 | 11.904 | −23.245 | 9.707 | 1.00 | 0.61 |
| ATOM | 857 | HB2 | SER | 57 | 11.291 | −22.142 | 8.479 | 1.00 | 0.57 |
| ATOM | 858 | OG | SER | 57 | 10.235 | −23.883 | 8.677 | 1.00 | 0.69 |
| ATOM | 859 | HG | SER | 57 | 9.561 | −23.481 | 8.123 | 1.00 | 1.15 |
| ATOM | 860 | C | SER | 57 | 11.078 | −21.070 | 11.006 | 1.00 | 0.50 |
| ATOM | 861 | O | SER | 57 | 11.730 | −20.304 | 10.328 | 1.00 | 0.45 |
| ATOM | 862 | N | PRO | 58 | 11.127 | −21.000 | 12.328 | 1.00 | 0.51 |
| ATOM | 863 | CA | PRO | 58 | 11.984 | −19.977 | 12.992 | 1.00 | 0.48 |
| ATOM | 864 | HA | PRO | 58 | 11.693 | −18.986 | 12.685 | 1.00 | 0.44 |
| ATOM | 865 | CB | PRO | 58 | 11.659 | −20.165 | 14.475 | 1.00 | 0.55 |
| ATOM | 866 | HB1 | PRO | 58 | 11.180 | −19.277 | 14.857 | 1.00 | 0.57 |
| ATOM | 867 | HB2 | PRO | 58 | 12.572 | −20.350 | 15.025 | 1.00 | 0.58 |
| ATOM | 868 | CG | PRO | 58 | 10.713 | −21.360 | 14.630 | 1.00 | 0.60 |
| ATOM | 869 | HG1 | PRO | 58 | 9.810 | −21.045 | 15.129 | 1.00 | 0.64 |
| ATOM | 870 | HG2 | PRO | 58 | 11.198 | −22.132 | 15.211 | 1.00 | 0.64 |
| ATOM | 871 | CD | PRO | 58 | 10.366 | −21.902 | 13.241 | 1.00 | 0.59 |
| ATOM | 872 | HD2 | PRO | 58 | 10.699 | −22.928 | 13.144 | 1.00 | 0.63 |
| ATOM | 873 | HD1 | PRO | 58 | 9.309 | −21.820 | 13.057 | 1.00 | 0.61 |
| ATOM | 874 | C | PRO | 58 | 13.479 | −20.198 | 12.738 | 1.00 | 0.47 |
| ATOM | 875 | O | PRO | 58 | 14.312 | −19.439 | 13.194 | 1.00 | 0.48 |
| ATOM | 876 | N | SER | 59 | 13.829 | −21.230 | 12.020 | 1.00 | 0.49 |
| ATOM | 877 | HN | SER | 59 | 13.146 | −21.836 | 11.663 | 1.00 | 0.51 |
| ATOM | 878 | CA | SER | 59 | 15.273 | −21.490 | 11.752 | 1.00 | 0.52 |
| ATOM | 879 | HA | SER | 59 | 15.856 | −21.166 | 12.601 | 1.00 | 0.54 |
| ATOM | 880 | CB | SER | 59 | 15.491 | −22.987 | 11.535 | 1.00 | 0.59 |
| ATOM | 881 | HB1 | SER | 59 | 15.026 | −23.539 | 12.341 | 1.00 | 0.97 |
| ATOM | 882 | HB2 | SER | 59 | 16.547 | −23.200 | 11.520 | 1.00 | 1.14 |
| ATOM | 883 | OG | SER | 59 | 14.920 | −23.368 | 10.291 | 1.00 | 1.30 |
| ATOM | 884 | HG | SER | 59 | 14.343 | −22.658 | 9.999 | 1.00 | 1.81 |
| ATOM | 885 | C | SER | 59 | 15.726 | −20.723 | 10.506 | 1.00 | 0.48 |
| ATOM | 886 | O | SER | 59 | 16.876 | −20.346 | 10.389 | 1.00 | 0.51 |
| ATOM | 887 | N | LYS | 60 | 14.844 | −20.487 | 9.571 | 1.00 | 0.46 |
| ATOM | 888 | HN | LYS | 60 | 13.920 | −20.797 | 9.674 | 1.00 | 0.46 |
| ATOM | 889 | CA | LYS | 60 | 15.257 | −19.746 | 8.344 | 1.00 | 0.45 |
| ATOM | 890 | HA | LYS | 60 | 16.288 | −19.968 | 8.117 | 1.00 | 0.49 |
| ATOM | 891 | CB | LYS | 60 | 14.372 | −20.154 | 7.165 | 1.00 | 0.49 |
| ATOM | 892 | HB1 | LYS | 60 | 14.552 | −19.487 | 6.335 | 1.00 | 0.51 |
| ATOM | 893 | HB2 | LYS | 60 | 13.338 | −20.085 | 7.460 | 1.00 | 0.46 |
| ATOM | 894 | CG | LYS | 60 | 14.671 | −21.589 | 6.731 | 1.00 | 0.57 |
| ATOM | 895 | HG1 | LYS | 60 | 14.342 | −22.275 | 7.496 | 1.00 | 0.82 |
| ATOM | 896 | HG2 | LYS | 60 | 15.734 | −21.705 | 6.570 | 1.00 | 1.07 |
| ATOM | 897 | CD | LYS | 60 | 13.917 | −21.874 | 5.428 | 1.00 | 1.20 |
| ATOM | 898 | HD1 | LYS | 60 | 14.502 | −21.522 | 4.592 | 1.00 | 1.80 |
| ATOM | 899 | HD2 | LYS | 60 | 12.968 | −21.357 | 5.444 | 1.00 | 1.78 |
| ATOM | 900 | CE | LYS | 60 | 13.675 | −23.376 | 5.280 | 1.00 | 1.67 |
| ATOM | 901 | HE1 | LYS | 60 | 13.104 | −23.560 | 4.381 | 1.00 | 2.00 |
| ATOM | 902 | HE2 | LYS | 60 | 13.124 | −23.739 | 6.135 | 1.00 | 2.17 |
| ATOM | 903 | NZ | LYS | 60 | 14.981 | −24.086 | 5.191 | 1.00 | 2.48 |
| ATOM | 904 | HZ1 | LYS | 60 | 14.935 | −24.804 | 4.441 | 1.00 | 2.80 |
| ATOM | 905 | HZ2 | LYS | 60 | 15.187 | −24.546 | 6.101 | 1.00 | 3.08 |
| ATOM | 906 | HZ3 | LYS | 60 | 15.732 | −23.403 | 4.968 | 1.00 | 2.81 |
| ATOM | 907 | C | LYS | 60 | 15.086 | −18.242 | 8.570 | 1.00 | 0.39 |
| ATOM | 908 | O | LYS | 60 | 15.656 | −17.432 | 7.867 | 1.00 | 0.40 |
| ATOM | 909 | N | LEU | 61 | 14.295 | −17.862 | 9.535 | 1.00 | 0.34 |
| ATOM | 910 | HN | LEU | 61 | 13.833 | −18.532 | 10.085 | 1.00 | 0.35 |
| ATOM | 911 | CA | LEU | 61 | 14.080 | −16.406 | 9.787 | 1.00 | 0.30 |
| ATOM | 912 | HA | LEU | 61 | 13.695 | −15.937 | 8.895 | 1.00 | 0.30 |
| ATOM | 913 | CB | LEU | 61 | 13.082 | −16.224 | 10.935 | 1.00 | 0.28 |
| ATOM | 914 | HB1 | LEU | 61 | 12.898 | −15.171 | 11.086 | 1.00 | 0.26 |
| ATOM | 915 | HB2 | LEU | 61 | 13.496 | −16.648 | 11.836 | 1.00 | 0.30 |
| ATOM | 916 | CG | LEU | 61 | 11.760 | −16.926 | 10.607 | 1.00 | 0.29 |
| ATOM | 917 | HG | LEU | 61 | 11.939 | −17.985 | 10.500 | 1.00 | 0.32 |
| ATOM | 918 | CD1 | LEU | 61 | 10.767 | −16.695 | 11.747 | 1.00 | 0.31 |
| ATOM | 919 | HD11 | LEU | 61 | 11.298 | −16.675 | 12.688 | 1.00 | 1.07 |
| ATOM | 920 | HD12 | LEU | 61 | 10.041 | −17.495 | 11.761 | 1.00 | 1.07 |
| ATOM | 921 | HD13 | LEU | 61 | 10.262 | −15.753 | 11.599 | 1.00 | 1.02 |
| ATOM | 922 | CD2 | LEU | 61 | 11.175 | −16.374 | 9.305 | 1.00 | 0.29 |
| ATOM | 923 | HD21 | LEU | 61 | 11.610 | −16.894 | 8.466 | 1.00 | 1.05 |
| ATOM | 924 | HD22 | LEU | 61 | 11.395 | −15.322 | 9.228 | 1.00 | 1.00 |
| ATOM | 925 | HD23 | LEU | 61 | 10.105 | −16.519 | 9.302 | 1.00 | 1.00 |
| ATOM | 926 | C | LEU | 61 | 15.406 | −15.745 | 10.173 | 1.00 | 0.32 |
| ATOM | 927 | O | LEU | 61 | 15.699 | −14.637 | 9.772 | 1.00 | 0.32 |
| ATOM | 928 | N | SER | 62 | 16.202 | −16.411 | 10.963 | 1.00 | 0.35 |
| ATOM | 929 | HN | SER | 62 | 15.939 | −17.299 | 11.284 | 1.00 | 0.37 |
| ATOM | 930 | CA | SER | 62 | 17.501 | −15.819 | 11.396 | 1.00 | 0.40 |
| ATOM | 931 | HA | SER | 62 | 17.295 | −14.972 | 12.033 | 1.00 | 0.39 |
| ATOM | 932 | CB | SER | 62 | 18.288 | −16.854 | 12.204 | 1.00 | 0.46 |
| ATOM | 933 | HB1 | SER | 62 | 19.339 | −16.786 | 11.954 | 1.00 | 0.81 |
| ATOM | 934 | HB2 | SER | 62 | 17.925 | −17.842 | 11.979 | 1.00 | 0.76 |
| ATOM | 935 | OG | SER | 62 | 18.101 | −16.602 | 13.590 | 1.00 | 0.90 |
| ATOM | 936 | HG | SER | 62 | 18.764 | −15.966 | 13.869 | 1.00 | 1.29 |
| ATOM | 937 | C | SER | 62 | 18.326 | −15.333 | 10.188 | 1.00 | 0.43 |
| ATOM | 938 | O | SER | 62 | 18.585 | −14.153 | 10.065 | 1.00 | 0.43 |
| ATOM | 939 | N | PRO | 63 | 18.749 | −16.208 | 9.298 | 1.00 | 0.46 |
| ATOM | 940 | CA | PRO | 63 | 19.553 | −15.775 | 8.121 | 1.00 | 0.51 |
| ATOM | 941 | HA | PRO | 63 | 20.579 | −15.618 | 8.406 | 1.00 | 0.56 |
| ATOM | 942 | CB | PRO | 63 | 19.467 | −16.990 | 7.197 | 1.00 | 0.56 |
| ATOM | 943 | HB1 | PRO | 63 | 20.459 | −17.291 | 6.900 | 1.00 | 0.66 |
| ATOM | 944 | HB2 | PRO | 63 | 18.885 | −16.738 | 6.321 | 1.00 | 0.58 |
| ATOM | 945 | CG | PRO | 63 | 18.790 | −18.137 | 7.955 | 1.00 | 0.52 |
| ATOM | 946 | HG1 | PRO | 63 | 19.453 | −18.988 | 7.993 | 1.00 | 0.57 |
| ATOM | 947 | HG2 | PRO | 63 | 17.876 | −18.411 | 7.450 | 1.00 | 0.50 |
| ATOM | 948 | CD | PRO | 63 | 18.476 | −17.669 | 9.379 | 1.00 | 0.48 |
| ATOM | 949 | HD2 | PRO | 63 | 17.445 | −17.871 | 9.625 | 1.00 | 0.43 |
| ATOM | 950 | HD1 | PRO | 63 | 19.145 | −18.138 | 10.080 | 1.00 | 0.52 |
| ATOM | 951 | C | PRO | 63 | 18.998 | −14.526 | 7.425 | 1.00 | 0.47 |
| ATOM | 952 | O | PRO | 63 | 19.684 | −13.534 | 7.280 | 1.00 | 0.48 |
| ATOM | 953 | N | LYS | 64 | 17.773 | −14.567 | 6.977 | 1.00 | 0.44 |
| ATOM | 954 | HN | LYS | 64 | 17.233 | −15.377 | 7.089 | 1.00 | 0.45 |
| ATOM | 955 | CA | LYS | 64 | 17.205 | −13.380 | 6.277 | 1.00 | 0.44 |
| ATOM | 956 | HA | LYS | 64 | 17.910 | −13.040 | 5.532 | 1.00 | 0.48 |
| ATOM | 957 | CB | LYS | 64 | 15.891 | −13.762 | 5.589 | 1.00 | 0.48 |
| ATOM | 958 | HB1 | LYS | 64 | 16.029 | −14.687 | 5.051 | 1.00 | 0.57 |
| ATOM | 959 | HB2 | LYS | 64 | 15.617 | −12.982 | 4.895 | 1.00 | 0.50 |
| ATOM | 960 | CG | LYS | 64 | 14.782 | −13.937 | 6.639 | 1.00 | 0.47 |
| ATOM | 961 | HG1 | LYS | 64 | 14.491 | −12.969 | 7.019 | 1.00 | 0.52 |
| ATOM | 962 | HG2 | LYS | 64 | 15.153 | −14.543 | 7.452 | 1.00 | 0.65 |
| ATOM | 963 | CD | LYS | 64 | 13.554 | −14.620 | 6.017 | 1.00 | 0.65 |
| ATOM | 964 | HD1 | LYS | 64 | 12.944 | −15.036 | 6.804 | 1.00 | 1.34 |
| ATOM | 965 | HD2 | LYS | 64 | 13.871 | −15.413 | 5.356 | 1.00 | 1.14 |
| ATOM | 966 | CE | LYS | 64 | 12.726 | −13.599 | 5.228 | 1.00 | 1.10 |
| ATOM | 967 | HE1 | LYS | 64 | 13.339 | −12.750 | 4.972 | 1.00 | 1.80 |
| ATOM | 968 | HE2 | LYS | 64 | 11.893 | −13.270 | 5.831 | 1.00 | 1.66 |
| ATOM | 969 | NZ | LYS | 64 | 12.214 | −14.237 | 3.982 | 1.00 | 1.78 |
| ATOM | 970 | HZ1 | LYS | 64 | 11.179 | −14.312 | 4.032 | 1.00 | 2.21 |
| ATOM | 971 | HZ2 | LYS | 64 | 12.627 | −15.187 | 3.885 | 1.00 | 2.30 |
| ATOM | 972 | HZ3 | LYS | 64 | 12.478 | −13.657 | 3.161 | 1.00 | 2.22 |
| ATOM | 973 | C | LYS | 64 | 16.958 | −12.252 | 7.282 | 1.00 | 0.37 |
| ATOM | 974 | O | LYS | 64 | 17.022 | −11.087 | 6.942 | 1.00 | 0.37 |
| ATOM | 975 | N | ALA | 65 | 16.675 | −12.578 | 8.515 | 1.00 | 0.35 |
| ATOM | 976 | HN | ALA | 65 | 16.623 | −13.522 | 8.781 | 1.00 | 0.37 |
| ATOM | 977 | CA | ALA | 65 | 16.426 | −11.505 | 9.517 | 1.00 | 0.31 |
| ATOM | 978 | HA | ALA | 65 | 15.572 | −10.919 | 9.210 | 1.00 | 0.31 |
| ATOM | 979 | CB | ALA | 65 | 16.150 | −12.132 | 10.884 | 1.00 | 0.33 |
| ATOM | 980 | HB1 | ALA | 65 | 16.918 | −12.856 | 11.109 | 1.00 | 1.02 |
| ATOM | 981 | HB2 | ALA | 65 | 15.187 | −12.621 | 10.868 | 1.00 | 1.03 |
| ATOM | 982 | HB3 | ALA | 65 | 16.150 | −11.362 | 11.640 | 1.00 | 1.02 |
| ATOM | 983 | C | ALA | 65 | 17.658 | −10.602 | 9.606 | 1.00 | 0.32 |
| ATOM | 984 | O | ALA | 65 | 17.550 | −9.392 | 9.575 | 1.00 | 0.31 |
| ATOM | 985 | N | LYS | 66 | 18.830 | −11.172 | 9.710 | 1.00 | 0.36 |
| ATOM | 986 | HN | LYS | 66 | 18.903 | −12.149 | 9.728 | 1.00 | 0.38 |
| ATOM | 987 | CA | LYS | 66 | 20.054 | −10.325 | 9.790 | 1.00 | 0.39 |
| ATOM | 988 | HA | LYS | 66 | 19.992 | −9.685 | 10.656 | 1.00 | 0.39 |
| ATOM | 989 | CB | LYS | 66 | 21.302 | −11.208 | 9.901 | 1.00 | 0.47 |
| ATOM | 990 | HB1 | LYS | 66 | 22.180 | −10.612 | 9.704 | 1.00 | 0.50 |
| ATOM | 991 | HB2 | LYS | 66 | 21.240 | −12.006 | 9.176 | 1.00 | 0.49 |
| ATOM | 992 | CG | LYS | 66 | 21.403 | −11.808 | 11.305 | 1.00 | 0.50 |
| ATOM | 993 | HG1 | LYS | 66 | 20.530 | −12.412 | 11.506 | 1.00 | 0.47 |
| ATOM | 994 | HG2 | LYS | 66 | 21.467 | −11.011 | 12.033 | 1.00 | 0.50 |
| ATOM | 995 | CD | LYS | 66 | 22.658 | −12.684 | 11.385 | 1.00 | 0.60 |
| ATOM | 996 | HD1 | LYS | 66 | 23.521 | −12.100 | 11.100 | 1.00 | 0.89 |
| ATOM | 997 | HD2 | LYS | 66 | 22.553 | −13.522 | 10.711 | 1.00 | 0.79 |
| ATOM | 998 | CE | LYS | 66 | 22.847 | −13.201 | 12.814 | 1.00 | 0.91 |
| ATOM | 999 | HE1 | LYS | 66 | 22.576 | −12.429 | 13.518 | 1.00 | 1.53 |
| ATOM | 1000 | HE2 | LYS | 66 | 23.881 | −13.474 | 12.962 | 1.00 | 1.37 |
| ATOM | 1001 | NZ | LYS | 66 | 21.985 | −14.396 | 13.031 | 1.00 | 1.77 |
| ATOM | 1002 | HZ1 | LYS | 66 | 22.475 | −15.068 | 13.655 | 1.00 | 2.25 |
| ATOM | 1003 | HZ2 | LYS | 66 | 21.787 | −14.852 | 12.117 | 1.00 | 2.26 |
| ATOM | 1004 | HZ3 | LYS | 66 | 21.092 | −14.104 | 13.474 | 1.00 | 2.32 |
| ATOM | 1005 | C | LYS | 66 | 20.169 | −9.465 | 8.533 | 1.00 | 0.39 |
| ATOM | 1006 | O | LYS | 66 | 20.497 | −8.298 | 8.599 | 1.00 | 0.39 |
| ATOM | 1007 | N | LYS | 67 | 19.911 | −10.030 | 7.385 | 1.00 | 0.40 |
| ATOM | 1008 | HN | LYS | 67 | 19.653 | −10.975 | 7.348 | 1.00 | 0.42 |
| ATOM | 1009 | CA | LYS | 67 | 20.019 | −9.233 | 6.133 | 1.00 | 0.42 |
| ATOM | 1010 | HA | LYS | 67 | 21.007 | −8.801 | 6.069 | 1.00 | 0.46 |
| ATOM | 1011 | CB | LYS | 67 | 19.789 | −10.150 | 4.931 | 1.00 | 0.47 |
| ATOM | 1012 | HB1 | LYS | 67 | 19.743 | −9.560 | 4.028 | 1.00 | 0.51 |
| ATOM | 1013 | HB2 | LYS | 67 | 18.860 | −10.688 | 5.059 | 1.00 | 0.45 |
| ATOM | 1014 | CG | LYS | 67 | 20.949 | −11.143 | 4.832 | 1.00 | 0.54 |
| ATOM | 1015 | HG1 | LYS | 67 | 20.996 | −11.732 | 5.735 | 1.00 | 0.73 |
| ATOM | 1016 | HG2 | LYS | 67 | 21.876 | −10.601 | 4.708 | 1.00 | 0.88 |
| ATOM | 1017 | CD | LYS | 67 | 20.737 | −12.070 | 3.634 | 1.00 | 1.02 |
| ATOM | 1018 | HD1 | LYS | 67 | 20.694 | −11.484 | 2.728 | 1.00 | 1.58 |
| ATOM | 1019 | HD2 | LYS | 67 | 19.811 | −12.612 | 3.758 | 1.00 | 1.44 |
| ATOM | 1020 | CE | LYS | 67 | 21.902 | −13.058 | 3.543 | 1.00 | 1.16 |
| ATOM | 1021 | HE1 | LYS | 67 | 21.922 | −13.672 | 4.432 | 1.00 | 1.68 |
| ATOM | 1022 | HE2 | LYS | 67 | 22.831 | −12.514 | 3.461 | 1.00 | 1.61 |
| ATOM | 1023 | NZ | LYS | 67 | 21.726 | −13.925 | 2.344 | 1.00 | 1.81 |
| ATOM | 1024 | HZ1 | LYS | 67 | 20.813 | −13.714 | 1.894 | 1.00 | 2.28 |
| ATOM | 1025 | HZ2 | LYS | 67 | 21.749 | −14.925 | 2.633 | 1.00 | 2.30 |
| ATOM | 1026 | HZ3 | LYS | 67 | 22.494 | −13.742 | 1.668 | 1.00 | 2.26 |
| ATOM | 1027 | C | LYS | 67 | 18.977 | −8.113 | 6.147 | 1.00 | 0.38 |
| ATOM | 1028 | O | LYS | 67 | 19.294 | −6.962 | 5.928 | 1.00 | 0.40 |
| ATOM | 1029 | N | ILE | 68 | 17.740 | −8.430 | 6.425 | 1.00 | 0.34 |
| ATOM | 1030 | HN | ILE | 68 | 17.500 | −9.361 | 6.616 | 1.00 | 0.34 |
| ATOM | 1031 | CA | ILE | 68 | 16.700 | −7.363 | 6.471 | 1.00 | 0.33 |
| ATOM | 1032 | HA | ILE | 68 | 16.701 | −6.825 | 5.535 | 1.00 | 0.36 |
| ATOM | 1033 | CB | ILE | 68 | 15.318 | −7.985 | 6.700 | 1.00 | 0.34 |
| ATOM | 1034 | HB | ILE | 68 | 15.379 | −8.690 | 7.516 | 1.00 | 0.34 |
| ATOM | 1035 | CG1 | ILE | 68 | 14.877 | −8.706 | 5.415 | 1.00 | 0.38 |
| ATOM | 1036 | HG11 | ILE | 68 | 15.722 | −9.227 | 4.992 | 1.00 | 0.39 |
| ATOM | 1037 | HG12 | ILE | 68 | 14.517 | −7.976 | 4.705 | 1.00 | 0.40 |
| ATOM | 1038 | CG2 | ILE | 68 | 14.317 | −6.879 | 7.055 | 1.00 | 0.37 |
| ATOM | 1039 | HG21 | ILE | 68 | 13.310 | −7.250 | 6.944 | 1.00 | 1.13 |
| ATOM | 1040 | HG22 | ILE | 68 | 14.462 | −6.036 | 6.399 | 1.00 | 1.06 |
| ATOM | 1041 | HG23 | ILE | 68 | 14.472 | −6.569 | 8.079 | 1.00 | 1.06 |
| ATOM | 1042 | CD1 | ILE | 68 | 13.757 | −9.716 | 5.710 | 1.00 | 0.41 |
| ATOM | 1043 | HD11 | ILE | 68 | 13.980 | −10.259 | 6.615 | 1.00 | 1.07 |
| ATOM | 1044 | HD12 | ILE | 68 | 13.681 | −10.412 | 4.888 | 1.00 | 1.07 |
| ATOM | 1045 | HD13 | ILE | 68 | 12.818 | −9.196 | 5.822 | 1.00 | 1.16 |
| ATOM | 1046 | C | ILE | 68 | 17.025 | −6.398 | 7.608 | 1.00 | 0.31 |
| ATOM | 1047 | O | ILE | 68 | 16.998 | −5.194 | 7.443 | 1.00 | 0.32 |
| ATOM | 1048 | N | TYR | 69 | 17.336 | −6.917 | 8.762 | 1.00 | 0.30 |
| ATOM | 1049 | HN | TYR | 69 | 17.354 | −7.890 | 8.874 | 1.00 | 0.31 |
| ATOM | 1050 | CA | TYR | 69 | 17.665 | −6.032 | 9.908 | 1.00 | 0.30 |
| ATOM | 1051 | HA | TYR | 69 | 16.818 | −5.405 | 10.128 | 1.00 | 0.31 |
| ATOM | 1052 | CB | TYR | 69 | 17.999 | −6.888 | 11.129 | 1.00 | 0.32 |
| ATOM | 1053 | HB1 | TYR | 69 | 18.840 | −7.526 | 10.902 | 1.00 | 0.34 |
| ATOM | 1054 | HB2 | TYR | 69 | 17.143 | −7.496 | 11.384 | 1.00 | 0.34 |
| ATOM | 1055 | CG | TYR | 69 | 18.346 | −5.995 | 12.296 | 1.00 | 0.32 |
| ATOM | 1056 | CD1 | TYR | 69 | 19.645 | −5.490 | 12.428 | 1.00 | 0.35 |
| ATOM | 1057 | HD1 | TYR | 69 | 20.395 | −5.731 | 11.689 | 1.00 | 0.38 |
| ATOM | 1058 | CD2 | TYR | 69 | 17.374 | −5.683 | 13.253 | 1.00 | 0.34 |
| ATOM | 1059 | HD2 | TYR | 69 | 16.372 | −6.074 | 13.152 | 1.00 | 0.36 |
| ATOM | 1060 | CE1 | TYR | 69 | 19.972 | −4.672 | 13.515 | 1.00 | 0.38 |
| ATOM | 1061 | HE1 | TYR | 69 | 20.974 | −4.283 | 13.617 | 1.00 | 0.42 |
| ATOM | 1062 | CE2 | TYR | 69 | 17.699 | −4.864 | 14.340 | 1.00 | 0.37 |
| ATOM | 1063 | HE2 | TYR | 69 | 16.949 | −4.623 | 15.078 | 1.00 | 0.41 |
| ATOM | 1064 | CZ | TYR | 69 | 18.999 | −4.359 | 14.472 | 1.00 | 0.38 |
| ATOM | 1065 | OH | TYR | 69 | 19.320 | −3.553 | 15.544 | 1.00 | 0.42 |
| ATOM | 1066 | HH | TYR | 69 | 20.265 | −3.385 | 15.516 | 1.00 | 1.03 |
| ATOM | 1067 | C | TYR | 69 | 18.875 | −5.168 | 9.555 | 1.00 | 0.31 |
| ATOM | 1068 | O | TYR | 69 | 18.864 | −3.965 | 9.721 | 1.00 | 0.33 |
| ATOM | 1069 | N | ASN | 70 | 19.922 | −5.778 | 9.072 | 1.00 | 0.33 |
| ATOM | 1070 | HN | ASN | 70 | 19.910 | −6.750 | 8.950 | 1.00 | 0.34 |
| ATOM | 1071 | CA | ASN | 70 | 21.137 | −4.999 | 8.710 | 1.00 | 0.36 |
| ATOM | 1072 | HA | ASN | 70 | 21.424 | −4.381 | 9.545 | 1.00 | 0.39 |
| ATOM | 1073 | CB | ASN | 70 | 22.282 | −5.958 | 8.376 | 1.00 | 0.40 |
| ATOM | 1074 | HB1 | ASN | 70 | 23.104 | −5.403 | 7.950 | 1.00 | 0.43 |
| ATOM | 1075 | HB2 | ASN | 70 | 21.938 | −6.695 | 7.665 | 1.00 | 0.39 |
| ATOM | 1076 | CG | ASN | 70 | 22.752 | −6.658 | 9.653 | 1.00 | 0.44 |
| ATOM | 1077 | OD1 | ASN | 70 | 22.635 | −6.116 | 10.734 | 1.00 | 1.05 |
| ATOM | 1078 | ND2 | ASN | 70 | 23.282 | −7.847 | 9.573 | 1.00 | 0.99 |
| ATOM | 1079 | HD21 | ASN | 70 | 23.376 | −8.285 | 8.702 | 1.00 | 1.65 |
| ATOM | 1080 | HD22 | ASN | 70 | 23.586 | −8.303 | 10.386 | 1.00 | 0.99 |
| ATOM | 1081 | C | ASN | 70 | 20.846 | −4.115 | 7.497 | 1.00 | 0.36 |
| ATOM | 1082 | O | ASN | 70 | 21.461 | −3.088 | 7.305 | 1.00 | 0.41 |
| ATOM | 1083 | N | GLU | 71 | 19.934 | −4.518 | 6.659 | 1.00 | 0.34 |
| ATOM | 1084 | HN | GLU | 71 | 19.460 | −5.361 | 6.816 | 1.00 | 0.33 |
| ATOM | 1085 | CA | GLU | 71 | 19.631 | −3.703 | 5.451 | 1.00 | 0.37 |
| ATOM | 1086 | HA | GLU | 71 | 20.542 | −3.239 | 5.103 | 1.00 | 0.41 |
| ATOM | 1087 | CB | GLU | 71 | 19.089 | −4.618 | 4.347 | 1.00 | 0.42 |
| ATOM | 1088 | HB1 | GLU | 71 | 18.117 | −4.990 | 4.633 | 1.00 | 0.89 |
| ATOM | 1089 | HB2 | GLU | 71 | 19.766 | −5.448 | 4.204 | 1.00 | 0.65 |
| ATOM | 1090 | CG | GLU | 71 | 18.967 | −3.832 | 3.039 | 1.00 | 1.01 |
| ATOM | 1091 | HG1 | GLU | 71 | 19.928 | −3.418 | 2.777 | 1.00 | 1.38 |
| ATOM | 1092 | HG2 | GLU | 71 | 18.251 | −3.033 | 3.165 | 1.00 | 1.52 |
| ATOM | 1093 | CD | GLU | 71 | 18.496 | −4.768 | 1.923 | 1.00 | 1.15 |
| ATOM | 1094 | OE1 | GLU | 71 | 18.168 | −4.270 | 0.858 | 1.00 | 1.71 |
| ATOM | 1095 | OE2 | GLU | 71 | 18.475 | −5.967 | 2.151 | 1.00 | 1.57 |
| ATOM | 1096 | C | GLU | 71 | 18.599 | −2.605 | 5.760 | 1.00 | 0.37 |
| ATOM | 1097 | O | GLU | 71 | 18.784 | −1.461 | 5.395 | 1.00 | 0.56 |
| ATOM | 1098 | N | PHE | 72 | 17.492 | −2.951 | 6.376 | 1.00 | 0.36 |
| ATOM | 1099 | HN | PHE | 72 | 17.343 | −3.887 | 6.625 | 1.00 | 0.50 |
| ATOM | 1100 | CA | PHE | 72 | 16.426 | −1.926 | 6.640 | 1.00 | 0.38 |
| ATOM | 1101 | HA | PHE | 72 | 16.558 | −1.101 | 5.959 | 1.00 | 0.42 |
| ATOM | 1102 | CB | PHE | 72 | 15.065 | −2.563 | 6.364 | 1.00 | 0.41 |
| ATOM | 1103 | HB1 | PHE | 72 | 14.283 | −1.905 | 6.712 | 1.00 | 0.45 |
| ATOM | 1104 | HB2 | PHE | 72 | 14.998 | −3.508 | 6.884 | 1.00 | 0.43 |
| ATOM | 1105 | CG | PHE | 72 | 14.904 | −2.793 | 4.880 | 1.00 | 0.39 |
| ATOM | 1106 | CD1 | PHE | 72 | 14.338 | −1.798 | 4.075 | 1.00 | 0.45 |
| ATOM | 1107 | HD1 | PHE | 72 | 14.019 | −0.865 | 4.514 | 1.00 | 0.51 |
| ATOM | 1108 | CD2 | PHE | 72 | 15.318 | −4.003 | 4.311 | 1.00 | 0.39 |
| ATOM | 1109 | HD2 | PHE | 72 | 15.757 | −4.768 | 4.931 | 1.00 | 0.42 |
| ATOM | 1110 | CE1 | PHE | 72 | 14.185 | −2.014 | 2.700 | 1.00 | 0.47 |
| ATOM | 1111 | HE1 | PHE | 72 | 13.748 | −1.246 | 2.078 | 1.00 | 0.54 |
| ATOM | 1112 | CE2 | PHE | 72 | 15.166 | −4.218 | 2.936 | 1.00 | 0.40 |
| ATOM | 1113 | HE2 | PHE | 72 | 15.485 | −5.152 | 2.497 | 1.00 | 0.43 |
| ATOM | 1114 | CZ | PHE | 72 | 14.599 | −3.224 | 2.131 | 1.00 | 0.43 |
| ATOM | 1115 | HZ | PHE | 72 | 14.480 | −3.391 | 1.070 | 1.00 | 0.46 |
| ATOM | 1116 | C | PHE | 72 | 16.428 | −1.382 | 8.083 | 1.00 | 0.37 |
| ATOM | 1117 | O | PHE | 72 | 16.004 | −0.265 | 8.308 | 1.00 | 0.45 |
| ATOM | 1118 | N | ILE | 73 | 16.844 | −2.144 | 9.065 | 1.00 | 0.33 |
| ATOM | 1119 | HN | ILE | 73 | 17.154 | −3.056 | 8.887 | 1.00 | 0.31 |
| ATOM | 1120 | CA | ILE | 73 | 16.790 | −1.622 | 10.476 | 1.00 | 0.35 |
| ATOM | 1121 | HA | ILE | 73 | 16.040 | −0.850 | 10.543 | 1.00 | 0.39 |
| ATOM | 1122 | CB | ILE | 73 | 16.417 | −2.762 | 11.430 | 1.00 | 0.34 |
| ATOM | 1123 | HB | ILE | 73 | 17.183 | −3.521 | 11.381 | 1.00 | 0.33 |
| ATOM | 1124 | CG1 | ILE | 73 | 15.064 | −3.376 | 11.022 | 1.00 | 0.35 |
| ATOM | 1125 | HG11 | ILE | 73 | 14.816 | −4.175 | 11.706 | 1.00 | 0.40 |
| ATOM | 1126 | HG12 | ILE | 73 | 15.145 | −3.778 | 10.024 | 1.00 | 0.35 |
| ATOM | 1127 | CG2 | ILE | 73 | 16.343 | −2.234 | 12.869 | 1.00 | 0.39 |
| ATOM | 1128 | HG21 | ILE | 73 | 17.257 | −2.477 | 13.389 | 1.00 | 1.05 |
| ATOM | 1129 | HG22 | ILE | 73 | 15.507 | −2.691 | 13.378 | 1.00 | 1.11 |
| ATOM | 1130 | HG23 | ILE | 73 | 16.212 | −1.162 | 12.854 | 1.00 | 1.12 |
| ATOM | 1131 | CD1 | ILE | 73 | 13.944 | −2.324 | 11.048 | 1.00 | 0.40 |
| ATOM | 1132 | HD11 | ILE | 73 | 14.174 | −1.548 | 11.760 | 1.00 | 1.07 |
| ATOM | 1133 | HD12 | ILE | 73 | 13.015 | −2.799 | 11.330 | 1.00 | 1.09 |
| ATOM | 1134 | HD13 | ILE | 73 | 13.838 | −1.890 | 10.065 | 1.00 | 1.13 |
| ATOM | 1135 | C | ILE | 73 | 18.141 | −1.048 | 10.913 | 1.00 | 0.38 |
| ATOM | 1136 | O | ILE | 73 | 18.235 | −0.385 | 11.928 | 1.00 | 0.40 |
| ATOM | 1137 | N | SER | 74 | 19.188 | −1.295 | 10.182 | 1.00 | 0.45 |
| ATOM | 1138 | HN | SER | 74 | 19.108 | −1.837 | 9.370 | 1.00 | 0.51 |
| ATOM | 1139 | CA | SER | 74 | 20.516 | −0.755 | 10.599 | 1.00 | 0.50 |
| ATOM | 1140 | HA | SER | 74 | 20.767 | −1.142 | 11.576 | 1.00 | 0.55 |
| ATOM | 1141 | CB | SER | 74 | 21.586 | −1.181 | 9.600 | 1.00 | 0.65 |
| ATOM | 1142 | HB1 | SER | 74 | 21.160 | −1.205 | 8.610 | 1.00 | 1.28 |
| ATOM | 1143 | HB2 | SER | 74 | 21.953 | −2.158 | 9.857 | 1.00 | 1.25 |
| ATOM | 1144 | OG | SER | 74 | 22.663 | −0.254 | 9.645 | 1.00 | 1.39 |
| ATOM | 1145 | HG | SER | 74 | 23.480 | −0.752 | 9.720 | 1.00 | 1.87 |
| ATOM | 1146 | C | SER | 74 | 20.479 | 0.771 | 10.656 | 1.00 | 0.43 |
| ATOM | 1147 | O | SER | 74 | 20.057 | 1.428 | 9.725 | 1.00 | 0.43 |
| ATOM | 1148 | N | VAL | 75 | 20.949 | 1.341 | 11.731 | 1.00 | 0.44 |
| ATOM | 1149 | HN | VAL | 75 | 21.307 | 0.793 | 12.460 | 1.00 | 0.48 |
| ATOM | 1150 | CA | VAL | 75 | 20.976 | 2.824 | 11.832 | 1.00 | 0.47 |
| ATOM | 1151 | HA | VAL | 75 | 19.974 | 3.213 | 11.719 | 1.00 | 0.47 |
| ATOM | 1152 | CB | VAL | 75 | 21.543 | 3.241 | 13.191 | 1.00 | 0.55 |
| ATOM | 1153 | HB | VAL | 75 | 21.665 | 4.314 | 13.216 | 1.00 | 0.65 |
| ATOM | 1154 | CG1 | VAL | 75 | 20.580 | 2.811 | 14.300 | 1.00 | 0.58 |
| ATOM | 1155 | HG11 | VAL | 75 | 20.495 | 1.735 | 14.307 | 1.00 | 1.22 |
| ATOM | 1156 | HG12 | VAL | 75 | 19.609 | 3.248 | 14.122 | 1.00 | 1.13 |
| ATOM | 1157 | HG13 | VAL | 75 | 20.957 | 3.149 | 15.254 | 1.00 | 1.18 |
| ATOM | 1158 | CG2 | VAL | 75 | 22.898 | 2.565 | 13.405 | 1.00 | 0.62 |
| ATOM | 1159 | HG21 | VAL | 75 | 23.659 | 3.319 | 13.546 | 1.00 | 1.17 |
| ATOM | 1160 | HG22 | VAL | 75 | 23.142 | 1.964 | 12.541 | 1.00 | 1.22 |
| ATOM | 1161 | HG23 | VAL | 75 | 22.851 | 1.933 | 14.280 | 1.00 | 1.19 |
| ATOM | 1162 | C | VAL | 75 | 21.865 | 3.363 | 10.711 | 1.00 | 0.51 |
| ATOM | 1163 | O | VAL | 75 | 21.825 | 4.529 | 10.374 | 1.00 | 0.58 |
| ATOM | 1164 | N | GLN | 76 | 22.672 | 2.511 | 10.135 | 1.00 | 0.53 |
| ATOM | 1165 | HN | GLN | 76 | 22.683 | 1.576 | 10.431 | 1.00 | 0.51 |
| ATOM | 1166 | CA | GLN | 76 | 23.575 | 2.948 | 9.032 | 1.00 | 0.64 |
| ATOM | 1167 | HA | GLN | 76 | 23.706 | 4.019 | 9.069 | 1.00 | 0.69 |
| ATOM | 1168 | CB | GLN | 76 | 24.932 | 2.257 | 9.185 | 1.00 | 0.75 |
| ATOM | 1169 | HB1 | GLN | 76 | 25.538 | 2.460 | 8.316 | 1.00 | 0.85 |
| ATOM | 1170 | HB2 | GLN | 76 | 24.782 | 1.191 | 9.279 | 1.00 | 0.74 |
| ATOM | 1171 | CG | GLN | 76 | 25.643 | 2.783 | 10.434 | 1.00 | 0.81 |
| ATOM | 1172 | HG1 | GLN | 76 | 25.021 | 2.617 | 11.300 | 1.00 | 0.85 |
| ATOM | 1173 | HG2 | GLN | 76 | 25.831 | 3.841 | 10.322 | 1.00 | 1.02 |
| ATOM | 1174 | CD | GLN | 76 | 26.971 | 2.043 | 10.613 | 1.00 | 1.29 |
| ATOM | 1175 | OE1 | GLN | 76 | 27.267 | 1.119 | 9.882 | 1.00 | 1.74 |
| ATOM | 1176 | NE2 | GLN | 76 | 27.786 | 2.408 | 11.564 | 1.00 | 1.77 |
| ATOM | 1177 | HE21 | GLN | 76 | 27.546 | 3.151 | 12.157 | 1.00 | 1.99 |
| ATOM | 1178 | HE22 | GLN | 76 | 28.638 | 1.940 | 11.686 | 1.00 | 2.18 |
| ATOM | 1179 | C | GLN | 76 | 22.959 | 2.549 | 7.688 | 1.00 | 0.61 |
| ATOM | 1180 | O | GLN | 76 | 23.519 | 2.798 | 6.639 | 1.00 | 0.71 |
| ATOM | 1181 | N | ALA | 77 | 21.812 | 1.927 | 7.712 | 1.00 | 0.52 |
| ATOM | 1182 | HN | ALA | 77 | 21.379 | 1.733 | 8.570 | 1.00 | 0.46 |
| ATOM | 1183 | CA | ALA | 77 | 21.160 | 1.506 | 6.438 | 1.00 | 0.53 |
| ATOM | 1184 | HA | ALA | 77 | 21.728 | 0.704 | 5.991 | 1.00 | 0.62 |
| ATOM | 1185 | CB | ALA | 77 | 19.736 | 1.022 | 6.723 | 1.00 | 0.51 |
| ATOM | 1186 | HB1 | ALA | 77 | 19.207 | 1.775 | 7.288 | 1.00 | 1.18 |
| ATOM | 1187 | HB2 | ALA | 77 | 19.772 | 0.106 | 7.290 | 1.00 | 1.14 |
| ATOM | 1188 | HB3 | ALA | 77 | 19.223 | 0.848 | 5.789 | 1.00 | 1.09 |
| ATOM | 1189 | C | ALA | 77 | 21.098 | 2.688 | 5.471 | 1.00 | 0.57 |
| ATOM | 1190 | O | ALA | 77 | 20.834 | 3.808 | 5.860 | 1.00 | 0.58 |
| ATOM | 1191 | N | THR | 78 | 21.329 | 2.445 | 4.210 | 1.00 | 0.64 |
| ATOM | 1192 | HN | THR | 78 | 21.531 | 1.533 | 3.916 | 1.00 | 0.68 |
| ATOM | 1193 | CA | THR | 78 | 21.269 | 3.553 | 3.218 | 1.00 | 0.71 |
| ATOM | 1194 | HA | THR | 78 | 21.864 | 4.385 | 3.566 | 1.00 | 0.78 |
| ATOM | 1195 | CB | THR | 78 | 21.807 | 3.063 | 1.871 | 1.00 | 0.82 |
| ATOM | 1196 | HB | THR | 78 | 21.908 | 3.900 | 1.197 | 1.00 | 0.89 |
| ATOM | 1197 | OG1 | THR | 78 | 20.904 | 2.115 | 1.320 | 1.00 | 0.80 |
| ATOM | 1198 | HG1 | THR | 78 | 20.897 | 1.344 | 1.891 | 1.00 | 1.14 |
| ATOM | 1199 | CG2 | THR | 78 | 23.175 | 2.409 | 2.071 | 1.00 | 0.94 |
| ATOM | 1200 | HG21 | THR | 78 | 23.866 | 3.135 | 2.475 | 1.00 | 1.58 |
| ATOM | 1201 | HG22 | THR | 78 | 23.545 | 2.050 | 1.122 | 1.00 | 1.33 |
| ATOM | 1202 | HG23 | THR | 78 | 23.081 | 1.581 | 2.758 | 1.00 | 1.29 |
| ATOM | 1203 | C | THR | 78 | 19.814 | 3.993 | 3.055 | 1.00 | 0.66 |
| ATOM | 1204 | O | THR | 78 | 19.532 | 5.083 | 2.597 | 1.00 | 0.74 |
| ATOM | 1205 | N | LYS | 79 | 18.891 | 3.145 | 3.431 | 1.00 | 0.58 |
| ATOM | 1206 | HN | LYS | 79 | 19.153 | 2.274 | 3.796 | 1.00 | 0.57 |
| ATOM | 1207 | CA | LYS | 79 | 17.443 | 3.488 | 3.310 | 1.00 | 0.59 |
| ATOM | 1208 | HA | LYS | 79 | 17.336 | 4.545 | 3.119 | 1.00 | 0.67 |
| ATOM | 1209 | CB | LYS | 79 | 16.826 | 2.697 | 2.150 | 1.00 | 0.64 |
| ATOM | 1210 | HB1 | LYS | 79 | 17.139 | 3.132 | 1.213 | 1.00 | 0.72 |
| ATOM | 1211 | HB2 | LYS | 79 | 15.749 | 2.738 | 2.223 | 1.00 | 0.67 |
| ATOM | 1212 | CG | LYS | 79 | 17.285 | 1.236 | 2.210 | 1.00 | 0.62 |
| ATOM | 1213 | HG1 | LYS | 79 | 16.974 | 0.793 | 3.143 | 1.00 | 0.58 |
| ATOM | 1214 | HG2 | LYS | 79 | 18.362 | 1.194 | 2.134 | 1.00 | 0.63 |
| ATOM | 1215 | CD | LYS | 79 | 16.664 | 0.456 | 1.049 | 1.00 | 0.75 |
| ATOM | 1216 | HD1 | LYS | 79 | 16.979 | 0.892 | 0.113 | 1.00 | 1.13 |
| ATOM | 1217 | HD2 | LYS | 79 | 15.586 | 0.499 | 1.122 | 1.00 | 1.16 |
| ATOM | 1218 | CE | LYS | 79 | 17.122 | −1.002 | 1.109 | 1.00 | 1.04 |
| ATOM | 1219 | HE1 | LYS | 79 | 16.646 | −1.495 | 1.943 | 1.00 | 1.68 |
| ATOM | 1220 | HE2 | LYS | 79 | 18.195 | −1.038 | 1.235 | 1.00 | 1.62 |
| ATOM | 1221 | NZ | LYS | 79 | 16.747 | −1.694 | −0.157 | 1.00 | 1.61 |
| ATOM | 1222 | HZ1 | LYS | 79 | 15.881 | −1.268 | −0.541 | 1.00 | 2.15 |
| ATOM | 1223 | HZ2 | LYS | 79 | 16.583 | −2.703 | 0.037 | 1.00 | 2.00 |
| ATOM | 1224 | HZ3 | LYS | 79 | 17.517 | −1.594 | −0.848 | 1.00 | 2.07 |
| ATOM | 1225 | C | LYS | 79 | 16.723 | 3.139 | 4.616 | 1.00 | 0.51 |
| ATOM | 1226 | O | LYS | 79 | 15.948 | 2.207 | 4.682 | 1.00 | 0.50 |
| ATOM | 1227 | N | GLU | 80 | 16.969 | 3.886 | 5.657 | 1.00 | 0.51 |
| ATOM | 1228 | HN | GLU | 80 | 17.594 | 4.638 | 5.585 | 1.00 | 0.55 |
| ATOM | 1229 | CA | GLU | 80 | 16.296 | 3.598 | 6.955 | 1.00 | 0.47 |
| ATOM | 1230 | HA | GLU | 80 | 16.552 | 2.602 | 7.282 | 1.00 | 0.47 |
| ATOM | 1231 | CB | GLU | 80 | 16.743 | 4.615 | 8.006 | 1.00 | 0.55 |
| ATOM | 1232 | HB1 | GLU | 80 | 16.129 | 4.512 | 8.888 | 1.00 | 0.55 |
| ATOM | 1233 | HB2 | GLU | 80 | 16.638 | 5.614 | 7.608 | 1.00 | 0.60 |
| ATOM | 1234 | CG | GLU | 80 | 18.205 | 4.365 | 8.377 | 1.00 | 0.62 |
| ATOM | 1235 | HG1 | GLU | 80 | 18.822 | 4.451 | 7.496 | 1.00 | 0.93 |
| ATOM | 1236 | HG2 | GLU | 80 | 18.306 | 3.373 | 8.794 | 1.00 | 0.82 |
| ATOM | 1237 | CD | GLU | 80 | 18.646 | 5.402 | 9.410 | 1.00 | 1.22 |
| ATOM | 1238 | OE1 | GLU | 80 | 19.839 | 5.528 | 9.622 | 1.00 | 1.84 |
| ATOM | 1239 | OE2 | GLU | 80 | 17.781 | 6.050 | 9.976 | 1.00 | 1.91 |
| ATOM | 1240 | C | GLU | 80 | 14.783 | 3.704 | 6.770 | 1.00 | 0.43 |
| ATOM | 1241 | O | GLU | 80 | 14.298 | 4.526 | 6.019 | 1.00 | 0.49 |
| ATOM | 1242 | N | VAL | 81 | 14.035 | 2.882 | 7.451 | 1.00 | 0.39 |
| ATOM | 1243 | HN | VAL | 81 | 14.449 | 2.228 | 8.052 | 1.00 | 0.41 |
| ATOM | 1244 | CA | VAL | 81 | 12.552 | 2.933 | 7.317 | 1.00 | 0.37 |
| ATOM | 1245 | HA | VAL | 81 | 12.286 | 3.377 | 6.369 | 1.00 | 0.41 |
| ATOM | 1246 | CB | VAL | 81 | 11.986 | 1.514 | 7.394 | 1.00 | 0.38 |
| ATOM | 1247 | HB | VAL | 81 | 10.927 | 1.560 | 7.604 | 1.00 | 0.40 |
| ATOM | 1248 | CG1 | VAL | 81 | 12.211 | 0.801 | 6.060 | 1.00 | 0.43 |
| ATOM | 1249 | HG11 | VAL | 81 | 12.464 | −0.233 | 6.243 | 1.00 | 1.14 |
| ATOM | 1250 | HG12 | VAL | 81 | 13.019 | 1.280 | 5.527 | 1.00 | 1.05 |
| ATOM | 1251 | HG13 | VAL | 81 | 11.309 | 0.851 | 5.468 | 1.00 | 1.13 |
| ATOM | 1252 | CG2 | VAL | 81 | 12.698 | 0.745 | 8.508 | 1.00 | 0.38 |
| ATOM | 1253 | HG21 | VAL | 81 | 12.756 | 1.363 | 9.392 | 1.00 | 0.98 |
| ATOM | 1254 | HG22 | VAL | 81 | 13.696 | 0.484 | 8.186 | 1.00 | 1.17 |
| ATOM | 1255 | HG23 | VAL | 81 | 12.146 | −0.156 | 8.734 | 1.00 | 1.05 |
| ATOM | 1256 | C | VAL | 81 | 11.974 | 3.772 | 8.457 | 1.00 | 0.37 |
| ATOM | 1257 | O | VAL | 81 | 12.532 | 3.837 | 9.535 | 1.00 | 0.38 |
| ATOM | 1258 | N | ASN | 82 | 10.865 | 4.420 | 8.231 | 1.00 | 0.41 |
| ATOM | 1259 | HN | ASN | 82 | 10.429 | 4.361 | 7.355 | 1.00 | 0.44 |
| ATOM | 1260 | CA | ASN | 82 | 10.267 | 5.255 | 9.309 | 1.00 | 0.45 |
| ATOM | 1261 | HA | ASN | 82 | 11.035 | 5.861 | 9.765 | 1.00 | 0.47 |
| ATOM | 1262 | CB | ASN | 82 | 9.188 | 6.160 | 8.713 | 1.00 | 0.55 |
| ATOM | 1263 | HB1 | ASN | 82 | 9.652 | 7.005 | 8.228 | 1.00 | 0.63 |
| ATOM | 1264 | HB2 | ASN | 82 | 8.536 | 6.510 | 9.501 | 1.00 | 0.61 |
| ATOM | 1265 | CC | ASN | 82 | 8.373 | 5.370 | 7.689 | 1.00 | 0.56 |
| ATOM | 1266 | OD1 | ASN | 82 | 8.264 | 4.164 | 7.781 | 1.00 | 1.10 |
| ATOM | 1267 | ND2 | ASN | 82 | 7.790 | 6.004 | 6.709 | 1.00 | 1.18 |
| ATOM | 1268 | HD21 | ASN | 82 | 7.876 | 6.977 | 6.634 | 1.00 | 1.85 |
| ATOM | 1269 | HD22 | ASN | 82 | 7.264 | 5.507 | 6.049 | 1.00 | 1.22 |
| ATOM | 1270 | C | ASN | 82 | 9.641 | 4.343 | 10.364 | 1.00 | 0.42 |
| ATOM | 1271 | O | ASN | 82 | 8.602 | 3.750 | 10.153 | 1.00 | 0.45 |
| ATOM | 1272 | N | LEU | 83 | 10.273 | 4.228 | 11.498 | 1.00 | 0.40 |
| ATOM | 1273 | HN | LEU | 83 | 11.110 | 4.717 | 11.640 | 1.00 | 0.41 |
| ATOM | 1274 | CA | LEU | 83 | 9.733 | 3.357 | 12.580 | 1.00 | 0.42 |
| ATOM | 1275 | HA | LEU | 83 | 8.664 | 3.264 | 12.468 | 1.00 | 0.46 |
| ATOM | 1276 | CB | LEU | 83 | 10.381 | 1.969 | 12.510 | 1.00 | 0.41 |
| ATOM | 1277 | HB1 | LEU | 83 | 10.164 | 1.424 | 13.417 | 1.00 | 0.43 |
| ATOM | 1278 | HB2 | LEU | 83 | 11.451 | 2.083 | 12.413 | 1.00 | 0.40 |
| ATOM | 1279 | CG | LEU | 83 | 9.846 | 1.190 | 11.302 | 1.00 | 0.45 |
| ATOM | 1280 | HG | LEU | 83 | 9.973 | 1.781 | 10.408 | 1.00 | 0.46 |
| ATOM | 1281 | CD1 | LEU | 83 | 10.631 | −0.114 | 11.154 | 1.00 | 0.49 |
| ATOM | 1282 | HD11 | LEU | 83 | 10.352 | −0.599 | 10.230 | 1.00 | 1.04 |
| ATOM | 1283 | HD12 | LEU | 83 | 10.407 | −0.765 | 11.985 | 1.00 | 1.16 |
| ATOM | 1284 | HD13 | LEU | 83 | 11.689 | 0.103 | 11.141 | 1.00 | 1.14 |
| ATOM | 1285 | CD2 | LEU | 83 | 8.358 | 0.868 | 11.496 | 1.00 | 0.51 |
| ATOM | 1286 | HD21 | LEU | 83 | 8.129 | 0.810 | 12.549 | 1.00 | 1.21 |
| ATOM | 1287 | HD22 | LEU | 83 | 8.132 | −0.079 | 11.028 | 1.00 | 1.10 |
| ATOM | 1288 | HD23 | LEU | 83 | 7.761 | 1.644 | 11.042 | 1.00 | 1.06 |
| ATOM | 1289 | C | LEU | 83 | 10.050 | 3.988 | 13.935 | 1.00 | 0.45 |
| ATOM | 1290 | O | LEU | 83 | 10.908 | 4.840 | 14.048 | 1.00 | 0.48 |
| ATOM | 1291 | N | ASP | 84 | 9.366 | 3.575 | 14.965 | 1.00 | 0.49 |
| ATOM | 1292 | HN | ASP | 84 | 8.679 | 2.886 | 14.853 | 1.00 | 0.50 |
| ATOM | 1293 | CA | ASP | 84 | 9.632 | 4.152 | 16.310 | 1.00 | 0.55 |
| ATOM | 1294 | HA | ASP | 84 | 9.636 | 5.231 | 16.247 | 1.00 | 0.61 |
| ATOM | 1295 | CB | ASP | 84 | 8.542 | 3.700 | 17.283 | 1.00 | 0.64 |
| ATOM | 1296 | HB1 | ASP | 84 | 8.735 | 4.117 | 18.260 | 1.00 | 1.28 |
| ATOM | 1297 | HB2 | ASP | 84 | 8.541 | 2.621 | 17.345 | 1.00 | 1.26 |
| ATOM | 1298 | CG | ASP | 84 | 7.178 | 4.184 | 16.786 | 1.00 | 1.25 |
| ATOM | 1299 | OD1 | ASP | 84 | 6.183 | 3.793 | 17.374 | 1.00 | 2.03 |
| ATOM | 1300 | OD2 | ASP | 84 | 7.152 | 4.927 | 15.819 | 1.00 | 2.00 |
| ATOM | 1301 | C | ASP | 84 | 10.994 | 3.662 | 16.807 | 1.00 | 0.50 |
| ATOM | 1302 | O | ASP | 84 | 11.517 | 2.672 | 16.335 | 1.00 | 0.46 |
| ATOM | 1303 | N | SER | 85 | 11.575 | 4.349 | 17.752 | 1.00 | 0.55 |
| ATOM | 1304 | HN | SER | 85 | 11.139 | 5.146 | 18.118 | 1.00 | 0.61 |
| ATOM | 1305 | CA | SER | 85 | 12.905 | 3.922 | 18.273 | 1.00 | 0.56 |
| ATOM | 1306 | HA | SER | 85 | 13.579 | 3.757 | 17.445 | 1.00 | 0.55 |
| ATOM | 1307 | CB | SER | 85 | 13.475 | 5.012 | 19.182 | 1.00 | 0.68 |
| ATOM | 1308 | HB1 | SER | 85 | 14.435 | 4.693 | 19.566 | 1.00 | 1.19 |
| ATOM | 1309 | HB2 | SER | 85 | 12.802 | 5.186 | 20.005 | 1.00 | 1.27 |
| ATOM | 1310 | OG | SER | 85 | 13.624 | 6.213 | 18.437 | 1.00 | 1.48 |
| ATOM | 1311 | HG | SER | 85 | 14.112 | 6.007 | 17.637 | 1.00 | 1.97 |
| ATOM | 1312 | C | SER | 85 | 12.749 | 2.625 | 19.067 | 1.00 | 0.51 |
| ATOM | 1313 | O | SER | 85 | 13.688 | 1.874 | 19.239 | 1.00 | 0.51 |
| ATOM | 1314 | N | CYS | 86 | 11.569 | 2.354 | 19.553 | 1.00 | 0.53 |
| ATOM | 1315 | HN | CYS | 86 | 10.824 | 2.972 | 19.403 | 1.00 | 0.56 |
| ATOM | 1316 | CA | CYS | 86 | 11.355 | 1.104 | 20.334 | 1.00 | 0.54 |
| ATOM | 1317 | HA | CYS | 86 | 12.225 | 0.903 | 20.940 | 1.00 | 0.58 |
| ATOM | 1318 | CB | CYS | 86 | 10.129 | 1.272 | 21.236 | 1.00 | 0.64 |
| ATOM | 1319 | HB1 | CYS | 86 | 9.326 | 0.650 | 20.869 | 1.00 | 1.29 |
| ATOM | 1320 | HB2 | CYS | 86 | 9.816 | 2.306 | 21.228 | 1.00 | 1.16 |
| ATOM | 1321 | SG | CYS | 86 | 10.548 | 0.782 | 22.927 | 1.00 | 1.74 |
| ATOM | 1322 | HG | CYS | 86 | 9.773 | 0.922 | 23.476 | 1.00 | 2.26 |
| ATOM | 1323 | C | CYS | 86 | 11.120 | −0.061 | 19.371 | 1.00 | 0.48 |
| ATOM | 1324 | O | CYS | 86 | 11.554 | −1.171 | 19.606 | 1.00 | 0.47 |
| ATOM | 1325 | N | THR | 87 | 10.433 | 0.183 | 18.290 | 1.00 | 0.45 |
| ATOM | 1326 | HN | THR | 87 | 10.091 | 1.086 | 18.122 | 1.00 | 0.48 |
| ATOM | 1327 | CA | THR | 87 | 10.167 | −0.910 | 17.314 | 1.00 | 0.42 |
| ATOM | 1328 | HA | THR | 87 | 9.615 | −1.701 | 17.800 | 1.00 | 0.46 |
| ATOM | 1329 | CB | THR | 87 | 9.344 | −0.356 | 16.145 | 1.00 | 0.45 |
| ATOM | 1330 | HB | THR | 87 | 9.912 | 0.401 | 15.628 | 1.00 | 0.45 |
| ATOM | 1331 | OG1 | THR | 87 | 8.140 | 0.208 | 16.647 | 1.00 | 0.50 |
| ATOM | 1332 | HG1 | THR | 87 | 7.466 | −0.476 | 16.638 | 1.00 | 0.94 |
| ATOM | 1333 | CG2 | THR | 87 | 9.000 | −1.485 | 15.169 | 1.00 | 0.48 |
| ATOM | 1334 | HG21 | THR | 87 | 9.908 | −1.938 | 14.800 | 1.00 | 1.14 |
| ATOM | 1335 | HG22 | THR | 87 | 8.439 | −1.083 | 14.340 | 1.00 | 1.02 |
| ATOM | 1336 | HG23 | THR | 87 | 8.406 | −2.231 | 15.677 | 1.00 | 1.19 |
| ATOM | 1337 | C | THR | 87 | 11.496 | −1.463 | 16.789 | 1.00 | 0.38 |
| ATOM | 1338 | O | THR | 87 | 11.717 | −2.658 | 16.771 | 1.00 | 0.38 |
| ATOM | 1339 | N | ARG | 88 | 12.383 | −0.606 | 16.354 | 1.00 | 0.37 |
| ATOM | 1340 | HN | ARG | 88 | 12.188 | 0.354 | 16.370 | 1.00 | 0.39 |
| ATOM | 1341 | CA | ARG | 88 | 13.689 | −1.094 | 15.825 | 1.00 | 0.37 |
| ATOM | 1342 | HA | ARG | 88 | 13.517 | −1.748 | 14.983 | 1.00 | 0.37 |
| ATOM | 1343 | CB | ARG | 88 | 14.545 | 0.094 | 15.382 | 1.00 | 0.41 |
| ATOM | 1344 | HB1 | ARG | 88 | 15.533 | −0.253 | 15.118 | 1.00 | 0.45 |
| ATOM | 1345 | HB2 | ARG | 88 | 14.619 | 0.805 | 16.192 | 1.00 | 0.43 |
| ATOM | 1346 | CG | ARG | 88 | 13.907 | 0.771 | 14.169 | 1.00 | 0.47 |
| ATOM | 1347 | HG1 | ARG | 88 | 13.000 | 1.271 | 14.470 | 1.00 | 0.85 |
| ATOM | 1348 | HG2 | ARG | 88 | 13.678 | 0.026 | 13.420 | 1.00 | 0.82 |
| ATOM | 1349 | CD | ARG | 88 | 14.885 | 1.795 | 13.590 | 1.00 | 0.93 |
| ATOM | 1350 | HD1 | ARG | 88 | 15.525 | 1.310 | 12.867 | 1.00 | 1.47 |
| ATOM | 1351 | HD2 | ARG | 88 | 15.489 | 2.206 | 14.384 | 1.00 | 1.51 |
| ATOM | 1352 | NE | ARG | 88 | 14.123 | 2.890 | 12.929 | 1.00 | 1.81 |
| ATOM | 1353 | HE | ARG | 88 | 13.166 | 2.783 | 12.748 | 1.00 | 2.38 |
| ATOM | 1354 | CZ | ARG | 88 | 14.728 | 4.002 | 12.610 | 1.00 | 2.49 |
| ATOM | 1355 | NH1 | ARG | 88 | 14.055 | 4.979 | 12.068 | 1.00 | 3.58 |
| ATOM | 1356 | HH11 | ARG | 88 | 13.075 | 4.877 | 11.896 | 1.00 | 3.99 |
| ATOM | 1357 | HH12 | ARG | 88 | 14.519 | 5.831 | 11.825 | 1.00 | 4.19 |
| ATOM | 1358 | NH2 | ARG | 88 | 16.008 | 4.134 | 12.829 | 1.00 | 2.58 |
| ATOM | 1359 | HH21 | ARG | 88 | 16.524 | 3.383 | 13.241 | 1.00 | 2.27 |
| ATOM | 1360 | HH22 | ARG | 88 | 16.472 | 4.985 | 12.585 | 1.00 | 3.35 |
| ATOM | 1361 | C | ARG | 88 | 14.433 | −1.857 | 16.921 | 1.00 | 0.37 |
| ATOM | 1362 | O | ARG | 88 | 14.927 | −2.947 | 16.707 | 1.00 | 0.36 |
| ATOM | 1363 | N | GLU | 89 | 14.521 | −1.292 | 18.094 | 1.00 | 0.40 |
| ATOM | 1364 | HN | GLU | 89 | 14.119 | −0.412 | 18.247 | 1.00 | 0.42 |
| ATOM | 1365 | CA | GLU | 89 | 15.238 | −1.987 | 19.199 | 1.00 | 0.43 |
| ATOM | 1366 | HA | GLU | 89 | 16.263 | −2.158 | 18.911 | 1.00 | 0.44 |
| ATOM | 1367 | CB | GLU | 89 | 15.198 | −1.120 | 20.459 | 1.00 | 0.49 |
| ATOM | 1368 | HB1 | GLU | 89 | 15.560 | −1.691 | 21.301 | 1.00 | 0.53 |
| ATOM | 1369 | HB2 | GLU | 89 | 14.182 | −0.806 | 20.648 | 1.00 | 0.50 |
| ATOM | 1370 | CG | GLU | 89 | 16.086 | 0.110 | 20.262 | 1.00 | 0.53 |
| ATOM | 1371 | HG1 | GLU | 89 | 15.724 | 0.684 | 19.422 | 1.00 | 0.73 |
| ATOM | 1372 | HG2 | GLU | 89 | 17.102 | −0.206 | 20.073 | 1.00 | 0.73 |
| ATOM | 1373 | CD | GLU | 89 | 16.046 | 0.975 | 21.523 | 1.00 | 0.95 |
| ATOM | 1374 | OE1 | GLU | 89 | 16.839 | 1.898 | 21.609 | 1.00 | 1.64 |
| ATOM | 1375 | OE2 | GLU | 89 | 15.223 | 0.700 | 22.380 | 1.00 | 1.54 |
| ATOM | 1376 | C | GLU | 89 | 14.559 | −3.325 | 19.479 | 1.00 | 0.41 |
| ATOM | 1377 | O | GLU | 89 | 15.207 | −4.326 | 19.711 | 1.00 | 0.42 |
| ATOM | 1378 | N | GLU | 90 | 13.257 | −3.352 | 19.456 | 1.00 | 0.42 |
| ATOM | 1379 | HN | GLU | 90 | 12.753 | −2.535 | 19.265 | 1.00 | 0.42 |
| ATOM | 1380 | CA | GLU | 90 | 12.542 | −4.628 | 19.717 | 1.00 | 0.44 |
| ATOM | 1381 | HA | GLU | 90 | 12.802 | −4.990 | 20.701 | 1.00 | 0.48 |
| ATOM | 1382 | CB | GLU | 90 | 11.032 | −4.395 | 19.645 | 1.00 | 0.49 |
| ATOM | 1383 | HB1 | GLU | 90 | 10.749 | −4.177 | 18.626 | 1.00 | 0.75 |
| ATOM | 1384 | HB2 | GLU | 90 | 10.766 | −3.563 | 20.280 | 1.00 | 0.91 |
| ATOM | 1385 | CG | GLU | 90 | 10.301 | −5.653 | 20.116 | 1.00 | 1.00 |
| ATOM | 1386 | HG1 | GLU | 90 | 10.670 | −5.941 | 21.090 | 1.00 | 1.59 |
| ATOM | 1387 | HG2 | GLU | 90 | 10.474 | −6.455 | 19.413 | 1.00 | 1.45 |
| ATOM | 1388 | CD | GLU | 90 | 8.802 | −5.368 | 20.207 | 1.00 | 1.19 |
| ATOM | 1389 | OE1 | GLU | 90 | 8.052 | −6.305 | 20.424 | 1.00 | 1.64 |
| ATOM | 1390 | OE2 | GLU | 90 | 8.429 | −4.215 | 20.058 | 1.00 | 1.85 |
| ATOM | 1391 | C | GLU | 90 | 12.958 | −5.663 | 18.671 | 1.00 | 0.40 |
| ATOM | 1392 | O | GLU | 90 | 13.167 | −6.820 | 18.979 | 1.00 | 0.43 |
| ATOM | 1393 | N | THR | 91 | 13.085 | −5.259 | 17.433 | 1.00 | 0.37 |
| ATOM | 1394 | HN | THR | 91 | 12.916 | −4.321 | 17.199 | 1.00 | 0.36 |
| ATOM | 1395 | CA | THR | 91 | 13.492 | −6.227 | 16.378 | 1.00 | 0.36 |
| ATOM | 1396 | HA | THR | 91 | 12.808 | −7.063 | 16.370 | 1.00 | 0.39 |
| ATOM | 1397 | CB | THR | 91 | 13.482 | −5.543 | 15.009 | 1.00 | 0.35 |
| ATOM | 1398 | HB | THR | 91 | 14.321 | −4.868 | 14.934 | 1.00 | 0.34 |
| ATOM | 1399 | OG1 | THR | 91 | 12.267 | −4.824 | 14.849 | 1.00 | 0.43 |
| ATOM | 1400 | HG1 | THR | 91 | 12.455 | −4.032 | 14.340 | 1.00 | 1.03 |
| ATOM | 1401 | CG2 | THR | 91 | 13.591 | −6.600 | 13.910 | 1.00 | 0.39 |
| ATOM | 1402 | HG21 | THR | 91 | 14.000 | −6.151 | 13.017 | 1.00 | 1.03 |
| ATOM | 1403 | HG22 | THR | 91 | 12.610 | −6.998 | 13.695 | 1.00 | 1.06 |
| ATOM | 1404 | HG23 | THR | 91 | 14.239 | −7.398 | 14.240 | 1.00 | 1.18 |
| ATOM | 1405 | C | THR | 91 | 14.904 | −6.726 | 16.678 | 1.00 | 0.36 |
| ATOM | 1406 | O | THR | 91 | 15.208 | −7.887 | 16.506 | 1.00 | 0.41 |
| ATOM | 1407 | N | SER | 92 | 15.770 | −5.850 | 17.119 | 1.00 | 0.35 |
| ATOM | 1408 | HN | SER | 92 | 15.498 | −4.916 | 17.243 | 1.00 | 0.34 |
| ATOM | 1409 | CA | SER | 92 | 17.170 | −6.263 | 17.422 | 1.00 | 0.39 |
| ATOM | 1410 | HA | SER | 92 | 17.634 | −6.646 | 16.526 | 1.00 | 0.41 |
| ATOM | 1411 | CB | SER | 92 | 17.957 | −5.050 | 17.920 | 1.00 | 0.42 |
| ATOM | 1412 | HB1 | SER | 92 | 17.820 | −4.227 | 17.231 | 1.00 | 0.43 |
| ATOM | 1413 | HB2 | SER | 92 | 19.004 | −5.296 | 17.977 | 1.00 | 0.51 |
| ATOM | 1414 | OG | SER | 92 | 17.490 | −4.685 | 19.212 | 1.00 | 0.40 |
| ATOM | 1415 | HG | SER | 92 | 16.642 | −5.112 | 19.353 | 1.00 | 0.98 |
| ATOM | 1416 | C | SER | 92 | 17.170 | −7.345 | 18.505 | 1.00 | 0.41 |
| ATOM | 1417 | O | SER | 92 | 17.917 | −8.300 | 18.443 | 1.00 | 0.47 |
| ATOM | 1418 | N | ARG | 93 | 16.344 | −7.202 | 19.502 | 1.00 | 0.40 |
| ATOM | 1419 | HN | ARG | 93 | 15.750 | −6.424 | 19.541 | 1.00 | 0.39 |
| ATOM | 1420 | CA | ARG | 93 | 16.305 | −8.228 | 20.580 | 1.00 | 0.45 |
| ATOM | 1421 | HA | ARG | 93 | 17.314 | −8.430 | 20.906 | 1.00 | 0.50 |
| ATOM | 1422 | CB | ARG | 93 | 15.486 | −7.706 | 21.767 | 1.00 | 0.49 |
| ATOM | 1423 | HB1 | ARG | 93 | 15.348 | −8.502 | 22.482 | 1.00 | 0.52 |
| ATOM | 1424 | HB2 | ARG | 93 | 14.523 | −7.360 | 21.419 | 1.00 | 0.47 |
| ATOM | 1425 | CG | ARG | 93 | 16.249 | −6.545 | 22.426 | 1.00 | 0.55 |
| ATOM | 1426 | HG1 | ARG | 93 | 16.224 | −5.689 | 21.768 | 1.00 | 0.81 |
| ATOM | 1427 | HG2 | ARG | 93 | 17.276 | −6.837 | 22.589 | 1.00 | 0.95 |
| ATOM | 1428 | CD | ARG | 93 | 15.616 | −6.163 | 23.771 | 1.00 | 1.01 |
| ATOM | 1429 | HD1 | ARG | 93 | 16.381 | −5.752 | 24.417 | 1.00 | 1.67 |
| ATOM | 1430 | HD2 | ARG | 93 | 15.192 | −7.035 | 24.239 | 1.00 | 1.59 |
| ATOM | 1431 | NE | ARG | 93 | 14.544 | −5.150 | 23.560 | 1.00 | 1.59 |
| ATOM | 1432 | HE | ARG | 93 | 14.468 | −4.684 | 22.702 | 1.00 | 2.15 |
| ATOM | 1433 | CZ | ARG | 93 | 13.713 | −4.876 | 24.530 | 1.00 | 2.23 |
| ATOM | 1434 | NH1 | ARG | 93 | 12.783 | −3.976 | 24.363 | 1.00 | 3.17 |
| ATOM | 1435 | HH11 | ARG | 93 | 12.705 | −3.492 | 23.491 | 1.00 | 3.53 |
| ATOM | 1436 | HH12 | ARG | 93 | 12.149 | −3.768 | 25.109 | 1.00 | 3.77 |
| ATOM | 1437 | NH2 | ARG | 93 | 13.817 | −5.502 | 25.671 | 1.00 | 2.56 |
| ATOM | 1438 | HH21 | ARG | 93 | 14.532 | −6.189 | 25.800 | 1.00 | 2.42 |
| ATOM | 1439 | HH22 | ARG | 93 | 13.182 | −5.294 | 26.415 | 1.00 | 3.34 |
| ATOM | 1440 | C | ARG | 93 | 15.696 | −9.526 | 20.034 | 1.00 | 0.44 |
| ATOM | 1441 | O | ARG | 93 | 16.049 | −10.611 | 20.450 | 1.00 | 0.46 |
| ATOM | 1442 | N | ASN | 94 | 14.781 | −9.419 | 19.108 | 1.00 | 0.43 |
| ATOM | 1443 | HN | ASN | 94 | 14.510 | −8.533 | 18.790 | 1.00 | 0.44 |
| ATOM | 1444 | CA | ASN | 94 | 14.142 | −10.642 | 18.535 | 1.00 | 0.46 |
| ATOM | 1445 | HA | ASN | 94 | 13.738 | −11.243 | 19.336 | 1.00 | 0.48 |
| ATOM | 1446 | CB | ASN | 94 | 13.012 | −10.233 | 17.589 | 1.00 | 0.52 |
| ATOM | 1447 | HB1 | ASN | 94 | 12.667 | −11.099 | 17.045 | 1.00 | 0.56 |
| ATOM | 1448 | HB2 | ASN | 94 | 13.376 | −9.491 | 16.893 | 1.00 | 0.53 |
| ATOM | 1449 | CG | ASN | 94 | 11.854 | −9.648 | 18.399 | 1.00 | 0.57 |
| ATOM | 1450 | OD1 | ASN | 94 | 11.720 | −9.922 | 19.575 | 1.00 | 1.33 |
| ATOM | 1451 | ND2 | ASN | 94 | 11.005 | −8.848 | 17.814 | 1.00 | 1.12 |
| ATOM | 1452 | HD21 | ASN | 94 | 11.114 | −8.628 | 16.865 | 1.00 | 1.88 |
| ATOM | 1453 | HD22 | ASN | 94 | 10.258 | −8.469 | 18.323 | 1.00 | 1.13 |
| ATOM | 1454 | C | ASN | 94 | 15.176 | −11.461 | 17.758 | 1.00 | 0.47 |
| ATOM | 1455 | O | ASN | 94 | 14.989 | −12.637 | 17.513 | 1.00 | 0.47 |
| ATOM | 1456 | N | MET | 95 | 16.261 | −10.856 | 17.362 | 1.00 | 0.55 |
| ATOM | 1457 | HN | MET | 95 | 16.396 | −9.907 | 17.564 | 1.00 | 0.59 |
| ATOM | 1458 | CA | MET | 95 | 17.292 | −11.613 | 16.597 | 1.00 | 0.63 |
| ATOM | 1459 | HA | MET | 95 | 16.860 | −11.996 | 15.684 | 1.00 | 0.66 |
| ATOM | 1460 | CB | MET | 95 | 18.466 | −10.691 | 16.267 | 1.00 | 0.78 |
| ATOM | 1461 | HB1 | MET | 95 | 19.130 | −11.185 | 15.573 | 1.00 | 0.83 |
| ATOM | 1462 | HB2 | MET | 95 | 19.003 | −10.451 | 17.173 | 1.00 | 0.87 |
| ATOM | 1463 | CG | MET | 95 | 17.932 | −9.409 | 15.632 | 1.00 | 0.92 |
| ATOM | 1464 | HG1 | MET | 95 | 18.755 | −8.745 | 15.415 | 1.00 | 1.48 |
| ATOM | 1465 | HG2 | MET | 95 | 17.259 | −8.930 | 16.317 | 1.00 | 1.45 |
| ATOM | 1466 | SD | MET | 95 | 17.048 | −9.799 | 14.104 | 1.00 | 1.27 |
| ATOM | 1467 | CE | MET | 95 | 18.402 | −9.445 | 12.963 | 1.00 | 0.68 |
| ATOM | 1468 | HE1 | MET | 95 | 18.054 | −9.569 | 11.950 | 1.00 | 1.31 |
| ATOM | 1469 | HE2 | MET | 95 | 19.218 | −10.123 | 13.148 | 1.00 | 1.10 |
| ATOM | 1470 | HE3 | MET | 95 | 18.740 | −8.429 | 13.110 | 1.00 | 1.19 |
| ATOM | 1471 | C | MET | 95 | 17.781 | −12.773 | 17.462 | 1.00 | 0.62 |
| ATOM | 1472 | O | MET | 95 | 18.093 | −13.840 | 16.973 | 1.00 | 0.65 |
| ATOM | 1473 | N | LEU | 96 | 17.838 | −12.570 | 18.749 | 1.00 | 0.62 |
| ATOM | 1474 | HN | LEU | 96 | 17.573 | −11.702 | 19.118 | 1.00 | 0.60 |
| ATOM | 1475 | CA | LEU | 96 | 18.290 | −13.657 | 19.660 | 1.00 | 0.69 |
| ATOM | 1476 | HA | LEU | 96 | 19.238 | −14.044 | 19.324 | 1.00 | 0.79 |
| ATOM | 1477 | CB | LEU | 96 | 18.420 | −13.104 | 21.084 | 1.00 | 0.74 |
| ATOM | 1478 | HB1 | LEU | 96 | 18.755 | −13.891 | 21.744 | 1.00 | 0.91 |
| ATOM | 1479 | HB2 | LEU | 96 | 17.458 | −12.743 | 21.417 | 1.00 | 0.71 |
| ATOM | 1480 | CG | LEU | 96 | 19.434 | −11.953 | 21.114 | 1.00 | 0.88 |
| ATOM | 1481 | HG | LEU | 96 | 19.332 | −11.359 | 20.217 | 1.00 | 1.46 |
| ATOM | 1482 | CD1 | LEU | 96 | 19.165 | −11.078 | 22.339 | 1.00 | 1.14 |
| ATOM | 1483 | HD11 | LEU | 96 | 18.289 | −10.471 | 22.164 | 1.00 | 1.78 |
| ATOM | 1484 | HD12 | LEU | 96 | 20.016 | −10.438 | 22.519 | 1.00 | 1.69 |
| ATOM | 1485 | HD13 | LEU | 96 | 18.999 | −11.707 | 23.201 | 1.00 | 1.48 |
| ATOM | 1486 | CD2 | LEU | 96 | 20.860 | −12.508 | 21.207 | 1.00 | 1.41 |
| ATOM | 1487 | HD21 | LEU | 96 | 21.562 | −11.688 | 21.231 | 1.00 | 1.93 |
| ATOM | 1488 | HD22 | LEU | 96 | 21.068 | −13.131 | 20.352 | 1.00 | 1.80 |
| ATOM | 1489 | HD23 | LEU | 96 | 20.961 | −13.091 | 22.111 | 1.00 | 1.98 |
| ATOM | 1490 | C | LEU | 96 | 17.244 | −14.774 | 19.639 | 1.00 | 0.63 |
| ATOM | 1491 | O | LEU | 96 | 17.546 | −15.930 | 19.858 | 1.00 | 0.72 |
| ATOM | 1492 | N | GLU | 97 | 16.013 | −14.426 | 19.374 | 1.00 | 0.54 |
| ATOM | 1493 | HN | GLU | 97 | 15.802 | −13.484 | 19.200 | 1.00 | 0.51 |
| ATOM | 1494 | CA | GLU | 97 | 14.925 | −15.445 | 19.329 | 1.00 | 0.52 |
| ATOM | 1495 | HA | GLU | 97 | 15.348 | −16.429 | 19.201 | 1.00 | 0.57 |
| ATOM | 1496 | CB | GLU | 97 | 14.134 | −15.397 | 20.638 | 1.00 | 0.61 |
| ATOM | 1497 | HB1 | GLU | 97 | 13.340 | −16.127 | 20.606 | 1.00 | 1.01 |
| ATOM | 1498 | HB2 | GLU | 97 | 13.713 | −14.411 | 20.769 | 1.00 | 1.07 |
| ATOM | 1499 | CG | GLU | 97 | 15.066 | −15.714 | 21.810 | 1.00 | 1.37 |
| ATOM | 1500 | HG1 | GLU | 97 | 15.579 | −14.814 | 22.116 | 1.00 | 1.99 |
| ATOM | 1501 | HG2 | GLU | 97 | 15.790 | −16.455 | 21.504 | 1.00 | 1.89 |
| ATOM | 1502 | CD | GLU | 97 | 14.246 | −16.254 | 22.984 | 1.00 | 1.82 |
| ATOM | 1503 | OE1 | GLU | 97 | 13.579 | −17.259 | 22.801 | 1.00 | 2.29 |
| ATOM | 1504 | OE2 | GLU | 97 | 14.300 | −15.654 | 24.044 | 1.00 | 2.43 |
| ATOM | 1505 | C | GLU | 97 | 13.987 | −15.114 | 18.160 | 1.00 | 0.43 |
| ATOM | 1506 | O | GLU | 97 | 12.921 | −14.565 | 18.357 | 1.00 | 0.44 |
| ATOM | 1507 | N | PRO | 98 | 14.382 | −15.427 | 16.946 | 1.00 | 0.40 |
| ATOM | 1508 | CA | PRO | 98 | 13.539 | −15.121 | 15.757 | 1.00 | 0.41 |
| ATOM | 1509 | HA | PRO | 98 | 13.400 | −14.057 | 15.660 | 1.00 | 0.45 |
| ATOM | 1510 | CB | PRO | 98 | 14.397 | −15.630 | 14.595 | 1.00 | 0.49 |
| ATOM | 1511 | HB1 | PRO | 98 | 14.543 | −14.838 | 13.874 | 1.00 | 0.56 |
| ATOM | 1512 | HB2 | PRO | 98 | 13.906 | −16.467 | 14.121 | 1.00 | 0.54 |
| ATOM | 1513 | CG | PRO | 98 | 15.758 | −16.073 | 15.146 | 1.00 | 0.50 |
| ATOM | 1514 | HG1 | PRO | 98 | 16.520 | −15.374 | 14.838 | 1.00 | 0.52 |
| ATOM | 1515 | HG2 | PRO | 98 | 15.996 | −17.060 | 14.774 | 1.00 | 0.57 |
| ATOM | 1516 | CD | PRO | 98 | 15.680 | −16.102 | 16.674 | 1.00 | 0.47 |
| ATOM | 1517 | HD2 | PRO | 98 | 15.669 | −17.124 | 17.029 | 1.00 | 0.53 |
| ATOM | 1518 | HD1 | PRO | 98 | 16.492 | −15.547 | 17.114 | 1.00 | 0.50 |
| ATOM | 1519 | C | PRO | 98 | 12.183 | −15.832 | 15.800 | 1.00 | 0.38 |
| ATOM | 1520 | O | PRO | 98 | 12.106 | −17.045 | 15.811 | 1.00 | 0.42 |
| ATOM | 1521 | N | THR | 99 | 11.118 | −15.073 | 15.829 | 1.00 | 0.37 |
| ATOM | 1522 | HN | THR | 99 | 11.220 | −14.098 | 15.823 | 1.00 | 0.38 |
| ATOM | 1523 | CA | THR | 99 | 9.748 | −15.668 | 15.878 | 1.00 | 0.38 |
| ATOM | 1524 | HA | THR | 99 | 9.809 | −16.742 | 15.786 | 1.00 | 0.43 |
| ATOM | 1525 | CB | THR | 99 | 9.076 | −15.304 | 17.205 | 1.00 | 0.45 |
| ATOM | 1526 | HB | THR | 99 | 8.084 | −15.727 | 17.233 | 1.00 | 0.46 |
| ATOM | 1527 | OG1 | THR | 99 | 8.989 | −13.891 | 17.316 | 1.00 | 0.49 |
| ATOM | 1528 | HG1 | THR | 99 | 9.097 | −13.517 | 16.438 | 1.00 | 0.85 |
| ATOM | 1529 | CG2 | THR | 99 | 9.892 | −15.861 | 18.374 | 1.00 | 0.58 |
| ATOM | 1530 | HG21 | THR | 99 | 9.862 | −15.163 | 19.198 | 1.00 | 1.21 |
| ATOM | 1531 | HG22 | THR | 99 | 10.915 | −16.007 | 18.064 | 1.00 | 1.22 |
| ATOM | 1532 | HG23 | THR | 99 | 9.473 | −16.806 | 18.687 | 1.00 | 1.10 |
| ATOM | 1533 | C | THR | 99 | 8.914 | −15.104 | 14.728 | 1.00 | 0.34 |
| ATOM | 1534 | O | THR | 99 | 9.319 | −14.181 | 14.051 | 1.00 | 0.30 |
| ATOM | 1535 | N | ILE | 100 | 7.751 | −15.649 | 14.503 | 1.00 | 0.37 |
| ATOM | 1536 | HN | ILE | 100 | 7.443 | −16.392 | 15.063 | 1.00 | 0.41 |
| ATOM | 1537 | CA | ILE | 100 | 6.890 | −15.143 | 13.398 | 1.00 | 0.38 |
| ATOM | 1538 | HA | ILE | 100 | 7.470 | −15.079 | 12.489 | 1.00 | 0.38 |
| ATOM | 1539 | CB | ILE | 100 | 5.713 | −16.102 | 13.190 | 1.00 | 0.46 |
| ATOM | 1540 | HB | ILE | 100 | 6.080 | −17.117 | 13.135 | 1.00 | 0.49 |
| ATOM | 1541 | CG1 | ILE | 100 | 4.970 | −15.744 | 11.895 | 1.00 | 0.53 |
| ATOM | 1542 | HG11 | ILE | 100 | 4.707 | −14.697 | 11.910 | 1.00 | 0.54 |
| ATOM | 1543 | HG12 | ILE | 100 | 4.070 | −16.337 | 11.826 | 1.00 | 0.59 |
| ATOM | 1544 | CG2 | ILE | 100 | 4.749 | −15.978 | 14.371 | 1.00 | 0.50 |
| ATOM | 1545 | HG21 | ILE | 100 | 4.105 | −16.844 | 14.403 | 1.00 | 1.03 |
| ATOM | 1546 | HG22 | ILE | 100 | 4.149 | −15.087 | 14.253 | 1.00 | 1.14 |
| ATOM | 1547 | HG23 | ILE | 100 | 5.312 | −15.913 | 15.290 | 1.00 | 1.17 |
| ATOM | 1548 | CD1 | ILE | 100 | 5.857 | −16.026 | 10.675 | 1.00 | 0.59 |
| ATOM | 1549 | HD11 | ILE | 100 | 6.567 | −16.804 | 10.910 | 1.00 | 1.22 |
| ATOM | 1550 | HD12 | ILE | 100 | 6.388 | −15.127 | 10.402 | 1.00 | 1.15 |
| ATOM | 1551 | HD13 | ILE | 100 | 5.238 | −16.342 | 9.848 | 1.00 | 1.11 |
| ATOM | 1552 | C | ILE | 100 | 6.365 | −13.754 | 13.767 | 1.00 | 0.38 |
| ATOM | 1553 | O | ILE | 100 | 5.956 | −12.988 | 12.917 | 1.00 | 0.41 |
| ATOM | 1554 | N | THR | 101 | 6.377 | −13.419 | 15.031 | 1.00 | 0.39 |
| ATOM | 1555 | HN | THR | 101 | 6.714 | −14.050 | 15.701 | 1.00 | 0.40 |
| ATOM | 1556 | CA | THR | 101 | 5.881 | −12.078 | 15.452 | 1.00 | 0.44 |
| ATOM | 1557 | HA | THR | 101 | 5.174 | −11.718 | 14.727 | 1.00 | 0.48 |
| ATOM | 1558 | CB | THR | 101 | 5.200 | −12.181 | 16.820 | 1.00 | 0.53 |
| ATOM | 1559 | HB | THR | 101 | 4.849 | −11.206 | 17.120 | 1.00 | 0.59 |
| ATOM | 1560 | OG1 | THR | 101 | 6.134 | −12.661 | 17.777 | 1.00 | 0.54 |
| ATOM | 1561 | HG1 | THR | 101 | 6.977 | −12.235 | 17.608 | 1.00 | 0.89 |
| ATOM | 1562 | CG2 | THR | 101 | 4.011 | −13.139 | 16.736 | 1.00 | 0.58 |
| ATOM | 1563 | HG21 | THR | 101 | 3.336 | −12.807 | 15.961 | 1.00 | 1.20 |
| ATOM | 1564 | HG22 | THR | 101 | 3.493 | −13.153 | 17.684 | 1.00 | 1.20 |
| ATOM | 1565 | HG23 | THR | 101 | 4.364 | −14.133 | 16.505 | 1.00 | 1.14 |
| ATOM | 1566 | C | THR | 101 | 7.058 | −11.104 | 15.546 | 1.00 | 0.41 |
| ATOM | 1567 | O | THR | 101 | 6.926 | −9.998 | 16.031 | 1.00 | 0.46 |
| ATOM | 1568 | N | CYS | 102 | 8.211 | −11.513 | 15.095 | 1.00 | 0.34 |
| ATOM | 1569 | HN | CYS | 102 | 8.295 | −12.412 | 14.714 | 1.00 | 0.31 |
| ATOM | 1570 | CA | CYS | 102 | 9.406 | −10.622 | 15.163 | 1.00 | 0.34 |
| ATOM | 1571 | HA | CYS | 102 | 9.591 | −10.353 | 16.192 | 1.00 | 0.42 |
| ATOM | 1572 | CB | CYS | 102 | 10.622 | −11.368 | 14.611 | 1.00 | 0.34 |
| ATOM | 1573 | HB1 | CYS | 102 | 10.377 | −11.795 | 13.650 | 1.00 | 0.33 |
| ATOM | 1574 | HB2 | CYS | 102 | 10.901 | −12.157 | 15.295 | 1.00 | 0.40 |
| ATOM | 1575 | SG | CYS | 102 | 12.004 | −10.214 | 14.425 | 1.00 | 0.37 |
| ATOM | 1576 | HG | CYS | 102 | 11.669 | −9.415 | 14.013 | 1.00 | 0.89 |
| ATOM | 1577 | C | CYS | 102 | 9.179 | −9.348 | 14.340 | 1.00 | 0.32 |
| ATOM | 1578 | O | CYS | 102 | 9.273 | −8.248 | 14.848 | 1.00 | 0.32 |
| ATOM | 1579 | N | PHE | 103 | 8.906 | −9.484 | 13.068 | 1.00 | 0.33 |
| ATOM | 1580 | HN | PHE | 103 | 8.852 | −10.379 | 12.673 | 1.00 | 0.36 |
| ATOM | 1581 | CA | PHE | 103 | 8.704 | −8.276 | 12.210 | 1.00 | 0.34 |
| ATOM | 1582 | HA | PHE | 103 | 9.226 | −7.437 | 12.643 | 1.00 | 0.34 |
| ATOM | 1583 | CB | PHE | 103 | 9.272 | −8.554 | 10.816 | 1.00 | 0.37 |
| ATOM | 1584 | HB1 | PHE | 103 | 8.966 | −7.770 | 10.140 | 1.00 | 0.41 |
| ATOM | 1585 | HB2 | PHE | 103 | 8.900 | −9.503 | 10.459 | 1.00 | 0.36 |
| ATOM | 1586 | CG | PHE | 103 | 10.780 | −8.599 | 10.881 | 1.00 | 0.38 |
| ATOM | 1587 | CD1 | PHE | 103 | 11.430 | −9.774 | 11.273 | 1.00 | 0.40 |
| ATOM | 1588 | HD1 | PHE | 103 | 10.854 | −10.647 | 11.537 | 1.00 | 0.45 |
| ATOM | 1589 | CD2 | PHE | 103 | 11.526 | −7.466 | 10.540 | 1.00 | 0.47 |
| ATOM | 1590 | HD2 | PHE | 103 | 11.022 | −6.561 | 10.233 | 1.00 | 0.56 |
| ATOM | 1591 | CE1 | PHE | 103 | 12.829 | −9.815 | 11.328 | 1.00 | 0.45 |
| ATOM | 1592 | HE1 | PHE | 103 | 13.331 | −10.722 | 11.630 | 1.00 | 0.52 |
| ATOM | 1593 | CE2 | PHE | 103 | 12.924 | −7.506 | 10.594 | 1.00 | 0.52 |
| ATOM | 1594 | HE2 | PHE | 103 | 13.500 | −6.630 | 10.333 | 1.00 | 0.62 |
| ATOM | 1595 | CZ | PHE | 103 | 13.576 | −8.680 | 10.989 | 1.00 | 0.48 |
| ATOM | 1596 | HZ | PHE | 103 | 14.655 | −8.712 | 11.030 | 1.00 | 0.53 |
| ATOM | 1597 | C | PHE | 103 | 7.216 | −7.935 | 12.081 | 1.00 | 0.35 |
| ATOM | 1598 | O | PHE | 103 | 6.856 | −6.949 | 11.469 | 1.00 | 0.35 |
| ATOM | 1599 | N | ASP | 104 | 6.344 | −8.728 | 12.637 | 1.00 | 0.37 |
| ATOM | 1600 | HN | ASP | 104 | 6.640 | −9.525 | 13.126 | 1.00 | 0.38 |
| ATOM | 1601 | CA | ASP | 104 | 4.890 | −8.415 | 12.516 | 1.00 | 0.41 |
| ATOM | 1602 | HA | ASP | 104 | 4.618 | −8.378 | 11.471 | 1.00 | 0.43 |
| ATOM | 1603 | CB | ASP | 104 | 4.063 | −9.488 | 13.220 | 1.00 | 0.47 |
| ATOM | 1604 | HB1 | ASP | 104 | 3.027 | −9.184 | 13.243 | 1.00 | 0.52 |
| ATOM | 1605 | HB2 | ASP | 104 | 4.423 | −9.613 | 14.228 | 1.00 | 0.45 |
| ATOM | 1606 | CG | ASP | 104 | 4.186 | −10.807 | 12.454 | 1.00 | 0.52 |
| ATOM | 1607 | OD1 | ASP | 104 | 4.780 | −10.796 | 11.388 | 1.00 | 1.11 |
| ATOM | 1608 | OD2 | ASP | 104 | 3.673 | −11.803 | 12.938 | 1.00 | 1.20 |
| ATOM | 1609 | C | ASP | 104 | 4.607 | −7.058 | 13.162 | 1.00 | 0.39 |
| ATOM | 1610 | O | ASP | 104 | 3.833 | −6.268 | 12.659 | 1.00 | 0.41 |
| ATOM | 1611 | N | GLU | 105 | 5.229 | −6.784 | 14.275 | 1.00 | 0.38 |
| ATOM | 1612 | HN | GLU | 105 | 5.847 | −7.438 | 14.663 | 1.00 | 0.38 |
| ATOM | 1613 | CA | GLU | 105 | 4.997 | −5.483 | 14.959 | 1.00 | 0.39 |
| ATOM | 1614 | HA | GLU | 105 | 3.942 | −5.367 | 15.160 | 1.00 | 0.42 |
| ATOM | 1615 | CB | GLU | 105 | 5.771 | −5.448 | 16.279 | 1.00 | 0.43 |
| ATOM | 1616 | HB1 | GLU | 105 | 6.831 | −5.442 | 16.075 | 1.00 | 0.85 |
| ATOM | 1617 | HB2 | GLU | 105 | 5.522 | −6.320 | 16.867 | 1.00 | 1.05 |
| ATOM | 1618 | CG | GLU | 105 | 5.398 | −4.183 | 17.056 | 1.00 | 1.06 |
| ATOM | 1619 | HG1 | GLU | 105 | 4.325 | −4.131 | 17.164 | 1.00 | 1.73 |
| ATOM | 1620 | HG2 | GLU | 105 | 5.750 | −3.314 | 16.519 | 1.00 | 1.61 |
| ATOM | 1621 | CD | GLU | 105 | 6.044 | −4.225 | 18.441 | 1.00 | 1.50 |
| ATOM | 1622 | OE1 | GLU | 105 | 5.950 | −3.235 | 19.148 | 1.00 | 2.16 |
| ATOM | 1623 | OE2 | GLU | 105 | 6.621 | −5.248 | 18.771 | 1.00 | 2.05 |
| ATOM | 1624 | C | GLU | 105 | 5.470 | −4.343 | 14.056 | 1.00 | 0.36 |
| ATOM | 1625 | O | GLU | 105 | 4.862 | −3.292 | 13.997 | 1.00 | 0.38 |
| ATOM | 1626 | N | ALA | 106 | 6.557 | −4.535 | 13.359 | 1.00 | 0.35 |
| ATOM | 1627 | HN | ALA | 106 | 7.039 | −5.386 | 13.425 | 1.00 | 0.35 |
| ATOM | 1628 | CA | ALA | 106 | 7.070 | −3.453 | 12.472 | 1.00 | 0.36 |
| ATOM | 1629 | HA | ALA | 106 | 7.149 | −2.536 | 13.034 | 1.00 | 0.37 |
| ATOM | 1630 | CB | ALA | 106 | 8.450 | −3.844 | 11.939 | 1.00 | 0.39 |
| ATOM | 1631 | HB1 | ALA | 106 | 9.160 | −3.066 | 12.176 | 1.00 | 1.00 |
| ATOM | 1632 | HB2 | ALA | 106 | 8.400 | −3.972 | 10.868 | 1.00 | 1.12 |
| ATOM | 1633 | HB3 | ALA | 106 | 8.764 | −4.770 | 12.399 | 1.00 | 1.10 |
| ATOM | 1634 | C | ALA | 106 | 6.110 | −3.247 | 11.296 | 1.00 | 0.36 |
| ATOM | 1635 | O | ALA | 106 | 5.792 | −2.130 | 10.939 | 1.00 | 0.36 |
| ATOM | 1636 | N | GLN | 107 | 5.632 | −4.304 | 10.698 | 1.00 | 0.41 |
| ATOM | 1637 | HN | GLN | 107 | 5.886 | −5.201 | 11.000 | 1.00 | 0.44 |
| ATOM | 1638 | CA | GLN | 107 | 4.683 | −4.135 | 9.560 | 1.00 | 0.45 |
| ATOM | 1639 | HA | GLN | 107 | 5.162 | −3.559 | 8.780 | 1.00 | 0.46 |
| ATOM | 1640 | CB | GLN | 107 | 4.263 | −5.498 | 9.004 | 1.00 | 0.54 |
| ATOM | 1641 | HB1 | GLN | 107 | 3.737 | −6.053 | 9.766 | 1.00 | 0.64 |
| ATOM | 1642 | HB2 | GLN | 107 | 5.141 | −6.048 | 8.697 | 1.00 | 0.68 |
| ATOM | 1643 | CG | GLN | 107 | 3.339 | −5.289 | 7.798 | 1.00 | 0.70 |
| ATOM | 1644 | HG1 | GLN | 107 | 3.863 | −4.733 | 7.035 | 1.00 | 0.87 |
| ATOM | 1645 | HG2 | GLN | 107 | 2.461 | −4.740 | 8.105 | 1.00 | 0.82 |
| ATOM | 1646 | CD | GLN | 107 | 2.913 | −6.643 | 7.233 | 1.00 | 0.81 |
| ATOM | 1647 | OE1 | GLN | 107 | 2.352 | −7.455 | 7.936 | 1.00 | 1.35 |
| ATOM | 1648 | NE2 | GLN | 107 | 3.158 | −6.922 | 5.982 | 1.00 | 1.26 |
| ATOM | 1649 | HE21 | GLN | 107 | 3.612 | −6.265 | 5.414 | 1.00 | 1.89 |
| ATOM | 1650 | HE22 | GLN | 107 | 2.888 | −7.788 | 5.611 | 1.00 | 1.28 |
| ATOM | 1651 | C | GLN | 107 | 3.455 | −3.378 | 10.063 | 1.00 | 0.43 |
| ATOM | 1652 | O | GLN | 107 | 2.921 | −2.520 | 9.389 | 1.00 | 0.44 |
| ATOM | 1653 | N | LYS | 108 | 3.008 | −3.688 | 11.249 | 1.00 | 0.44 |
| ATOM | 1654 | HN | LYS | 108 | 3.458 | −4.381 | 11.776 | 1.00 | 0.44 |
| ATOM | 1655 | CA | LYS | 108 | 1.819 | −2.986 | 11.805 | 1.00 | 0.47 |
| ATOM | 1656 | HA | LYS | 108 | 0.959 | −3.170 | 11.178 | 1.00 | 0.53 |
| ATOM | 1657 | CB | LYS | 108 | 1.551 | −3.505 | 13.223 | 1.00 | 0.51 |
| ATOM | 1658 | HB1 | LYS | 108 | 2.414 | −3.313 | 13.843 | 1.00 | 0.47 |
| ATOM | 1659 | HB2 | LYS | 108 | 1.366 | −4.568 | 13.185 | 1.00 | 0.55 |
| ATOM | 1660 | CG | LYS | 108 | 0.331 | −2.798 | 13.823 | 1.00 | 0.58 |
| ATOM | 1661 | HG1 | LYS | 108 | −0.536 | −2.991 | 13.209 | 1.00 | 0.64 |
| ATOM | 1662 | HG2 | LYS | 108 | 0.513 | −1.734 | 13.867 | 1.00 | 0.56 |
| ATOM | 1663 | CD | LYS | 108 | 0.080 | −3.329 | 15.236 | 1.00 | 0.66 |
| ATOM | 1664 | HD1 | LYS | 108 | 1.000 | −3.300 | 15.801 | 1.00 | 1.17 |
| ATOM | 1665 | HD2 | LYS | 108 | −0.275 | −4.348 | 15.180 | 1.00 | 0.97 |
| ATOM | 1666 | CE | LYS | 108 | −0.969 | −2.459 | 15.931 | 1.00 | 1.17 |
| ATOM | 1667 | HE1 | LYS | 108 | −1.535 | −1.917 | 15.189 | 1.00 | 1.70 |
| ATOM | 1668 | HE2 | LYS | 108 | −0.476 | −1.760 | 16.590 | 1.00 | 1.60 |
| ATOM | 1669 | NZ | LYS | 108 | −1.889 | −3.324 | 16.724 | 1.00 | 2.14 |
| ATOM | 1670 | HZ1 | LYS | 108 | −2.836 | −2.895 | 16.744 | 1.00 | 2.66 |
| ATOM | 1671 | HZ2 | LYS | 108 | −1.527 | −3.413 | 17.696 | 1.00 | 2.61 |
| ATOM | 1672 | HZ3 | LYS | 108 | −1.945 | −4.264 | 16.286 | 1.00 | 2.59 |
| ATOM | 1673 | C | LYS | 108 | 2.117 | −1.489 | 11.852 | 1.00 | 0.43 |
| ATOM | 1674 | O | LYS | 108 | 1.274 | −0.668 | 11.548 | 1.00 | 0.47 |
| ATOM | 1675 | N | LYS | 109 | 3.314 | −1.128 | 12.216 | 1.00 | 0.37 |
| ATOM | 1676 | HN | LYS | 109 | 3.983 | −1.806 | 12.448 | 1.00 | 0.35 |
| ATOM | 1677 | CA | LYS | 109 | 3.668 | 0.315 | 12.266 | 1.00 | 0.38 |
| ATOM | 1678 | HA | LYS | 109 | 2.945 | 0.838 | 12.867 | 1.00 | 0.43 |
| ATOM | 1679 | CB | LYS | 109 | 5.068 | 0.483 | 12.867 | 1.00 | 0.38 |
| ATOM | 1680 | HB1 | LYS | 109 | 5.348 | 1.525 | 12.835 | 1.00 | 0.66 |
| ATOM | 1681 | HB2 | LYS | 109 | 5.776 | −0.096 | 12.293 | 1.00 | 0.65 |
| ATOM | 1682 | CG | LYS | 109 | 5.076 | 0.000 | 14.326 | 1.00 | 0.71 |
| ATOM | 1683 | HG1 | LYS | 109 | 6.091 | −0.217 | 14.623 | 1.00 | 1.11 |
| ATOM | 1684 | HG2 | LYS | 109 | 4.482 | −0.898 | 14.406 | 1.00 | 1.07 |
| ATOM | 1685 | CD | LYS | 109 | 4.496 | 1.075 | 15.256 | 1.00 | 0.60 |
| ATOM | 1686 | HD1 | LYS | 109 | 3.471 | 1.278 | 14.994 | 1.00 | 0.54 |
| ATOM | 1687 | HD2 | LYS | 109 | 5.076 | 1.981 | 15.167 | 1.00 | 0.70 |
| ATOM | 1688 | CE | LYS | 109 | 4.549 | 0.574 | 16.701 | 1.00 | 1.05 |
| ATOM | 1689 | HE1 | LYS | 109 | 4.897 | −0.449 | 16.714 | 1.00 | 1.64 |
| ATOM | 1690 | HE2 | LYS | 109 | 3.561 | 0.623 | 17.134 | 1.00 | 1.55 |
| ATOM | 1691 | NZ | LYS | 109 | 5.480 | 1.426 | 17.493 | 1.00 | 1.71 |
| ATOM | 1692 | HZ1 | LYS | 109 | 5.381 | 1.202 | 18.503 | 1.00 | 2.20 |
| ATOM | 1693 | HZ2 | LYS | 109 | 5.251 | 2.429 | 17.334 | 1.00 | 2.20 |
| ATOM | 1694 | HZ3 | LYS | 109 | 6.458 | 1.241 | 17.194 | 1.00 | 2.19 |
| ATOM | 1695 | C | LYS | 109 | 3.631 | 0.880 | 10.845 | 1.00 | 0.37 |
| ATOM | 1696 | O | LYS | 109 | 3.171 | 1.983 | 10.622 | 1.00 | 0.39 |
| ATOM | 1697 | N | ILE | 110 | 4.099 | 0.135 | 9.876 | 1.00 | 0.35 |
| ATOM | 1698 | HN | ILE | 110 | 4.459 | −0.758 | 10.068 | 1.00 | 0.34 |
| ATOM | 1699 | CA | ILE | 110 | 4.064 | 0.646 | 8.478 | 1.00 | 0.36 |
| ATOM | 1700 | HA | ILE | 110 | 4.532 | 1.618 | 8.431 | 1.00 | 0.37 |
| ATOM | 1701 | CB | ILE | 110 | 4.791 | −0.332 | 7.549 | 1.00 | 0.38 |
| ATOM | 1702 | HB | ILE | 110 | 4.301 | −1.294 | 7.601 | 1.00 | 0.39 |
| ATOM | 1703 | CG1 | ILE | 110 | 6.258 | −0.485 | 7.991 | 1.00 | 0.40 |
| ATOM | 1704 | HG11 | ILE | 110 | 6.766 | −1.157 | 7.315 | 1.00 | 0.42 |
| ATOM | 1705 | HG12 | ILE | 110 | 6.283 | −0.902 | 8.987 | 1.00 | 0.41 |
| ATOM | 1706 | CG2 | ILE | 110 | 4.725 | 0.180 | 6.106 | 1.00 | 0.41 |
| ATOM | 1707 | HG21 | ILE | 110 | 5.675 | 0.018 | 5.620 | 1.00 | 1.14 |
| ATOM | 1708 | HG22 | ILE | 110 | 4.498 | 1.237 | 6.109 | 1.00 | 1.15 |
| ATOM | 1709 | HG23 | ILE | 110 | 3.954 | −0.352 | 5.570 | 1.00 | 1.01 |
| ATOM | 1710 | CD1 | ILE | 110 | 6.982 | 0.871 | 7.997 | 1.00 | 0.43 |
| ATOM | 1711 | HD11 | ILE | 110 | 6.630 | 1.485 | 7.183 | 1.00 | 1.15 |
| ATOM | 1712 | HD12 | ILE | 110 | 8.044 | 0.708 | 7.886 | 1.00 | 1.09 |
| ATOM | 1713 | HD13 | ILE | 110 | 6.795 | 1.374 | 8.933 | 1.00 | 1.08 |
| ATOM | 1714 | C | ILE | 110 | 2.600 | 0.762 | 8.050 | 1.00 | 0.36 |
| ATOM | 1715 | O | ILE | 110 | 2.194 | 1.741 | 7.457 | 1.00 | 0.37 |
| ATOM | 1716 | N | PHE | 111 | 1.798 | −0.223 | 8.367 | 1.00 | 0.37 |
| ATOM | 1717 | HN | PHE | 111 | 2.144 | −0.998 | 8.858 | 1.00 | 0.38 |
| ATOM | 1718 | CA | PHE | 111 | 0.353 | −0.160 | 8.002 | 1.00 | 0.39 |
| ATOM | 1719 | HA | PHE | 111 | 0.256 | −0.074 | 6.931 | 1.00 | 0.40 |
| ATOM | 1720 | CB | PHE | 111 | −0.357 | −1.434 | 8.478 | 1.00 | 0.41 |
| ATOM | 1721 | HB1 | PHE | 111 | −1.317 | −1.174 | 8.897 | 1.00 | 0.43 |
| ATOM | 1722 | HB2 | PHE | 111 | 0.245 | −1.919 | 9.232 | 1.00 | 0.43 |
| ATOM | 1723 | CG | PHE | 111 | −0.556 | −2.375 | 7.310 | 1.00 | 0.44 |
| ATOM | 1724 | CD1 | PHE | 111 | 0.521 | −2.699 | 6.477 | 1.00 | 0.47 |
| ATOM | 1725 | HD1 | PHE | 111 | 1.499 | −2.283 | 6.670 | 1.00 | 0.50 |
| ATOM | 1726 | CD2 | PHE | 111 | −1.823 | −2.918 | 7.056 | 1.00 | 0.46 |
| ATOM | 1727 | HD2 | PHE | 111 | −2.656 | −2.671 | 7.698 | 1.00 | 0.49 |
| ATOM | 1728 | CE1 | PHE | 111 | 0.330 | −3.563 | 5.390 | 1.00 | 0.52 |
| ATOM | 1729 | HE1 | PHE | 111 | 1.161 | −3.813 | 4.749 | 1.00 | 0.58 |
| ATOM | 1730 | CE2 | PHE | 111 | −2.011 | −3.781 | 5.969 | 1.00 | 0.50 |
| ATOM | 1731 | HE2 | PHE | 111 | −2.984 | −4.196 | 5.767 | 1.00 | 0.54 |
| ATOM | 1732 | CZ | PHE | 111 | −0.940 | −4.102 | 5.139 | 1.00 | 0.52 |
| ATOM | 1733 | HZ | PHE | 111 | −1.096 | −4.766 | 4.303 | 1.00 | 0.56 |
| ATOM | 1734 | C | PHE | 111 | −0.286 | 1.060 | 8.672 | 1.00 | 0.39 |
| ATOM | 1735 | O | PHE | 111 | −0.997 | 1.821 | 8.049 | 1.00 | 0.40 |
| ATOM | 1736 | N | ASN | 112 | −0.047 | 1.252 | 9.940 | 1.00 | 0.40 |
| ATOM | 1737 | HN | ASN | 112 | 0.527 | 0.627 | 10.434 | 1.00 | 0.41 |
| ATOM | 1738 | CA | ASN | 112 | −0.645 | 2.425 | 10.638 | 1.00 | 0.43 |
| ATOM | 1739 | HA | ASN | 112 | −1.713 | 2.431 | 10.473 | 1.00 | 0.44 |
| ATOM | 1740 | CB | ASN | 112 | −0.369 | 2.322 | 12.139 | 1.00 | 0.49 |
| ATOM | 1741 | HB1 | ASN | 112 | −0.478 | 3.295 | 12.592 | 1.00 | 0.51 |
| ATOM | 1742 | HB2 | ASN | 112 | 0.638 | 1.963 | 12.294 | 1.00 | 0.49 |
| ATOM | 1743 | CG | ASN | 112 | −1.363 | 1.350 | 12.777 | 1.00 | 0.54 |
| ATOM | 1744 | OD1 | ASN | 112 | −2.285 | 1.763 | 13.452 | 1.00 | 1.23 |
| ATOM | 1745 | ND2 | ASN | 112 | −1.218 | 0.067 | 12.587 | 1.00 | 1.22 |
| ATOM | 1746 | HD21 | ASN | 112 | −0.477 | −0.267 | 12.039 | 1.00 | 2.00 |
| ATOM | 1747 | HD22 | ASN | 112 | −1.850 | −0.562 | 12.992 | 1.00 | 1.25 |
| ATOM | 1748 | C | ASN | 112 | −0.044 | 3.729 | 10.100 | 1.00 | 0.42 |
| ATOM | 1749 | O | ASN | 112 | −0.734 | 4.712 | 9.922 | 1.00 | 0.43 |
| ATOM | 1750 | N | LEU | 113 | 1.241 | 3.755 | 9.861 | 1.00 | 0.42 |
| ATOM | 1751 | HN | LEU | 113 | 1.788 | 2.959 | 10.025 | 1.00 | 0.42 |
| ATOM | 1752 | CA | LEU | 113 | 1.877 | 5.011 | 9.361 | 1.00 | 0.45 |
| ATOM | 1753 | HA | LEU | 113 | 1.674 | 5.806 | 10.058 | 1.00 | 0.48 |
| ATOM | 1754 | CB | LEU | 113 | 3.396 | 4.800 | 9.246 | 1.00 | 0.51 |
| ATOM | 1755 | HB1 | LEU | 113 | 3.592 | 4.029 | 8.516 | 1.00 | 0.58 |
| ATOM | 1756 | HB2 | LEU | 113 | 3.784 | 4.487 | 10.204 | 1.00 | 0.65 |
| ATOM | 1757 | CG | LEU | 113 | 4.107 | 6.097 | 8.812 | 1.00 | 0.66 |
| ATOM | 1758 | HG | LEU | 113 | 3.530 | 6.596 | 8.049 | 1.00 | 1.18 |
| ATOM | 1759 | CD1 | LEU | 113 | 4.272 | 7.037 | 10.015 | 1.00 | 1.30 |
| ATOM | 1760 | HD11 | LEU | 113 | 4.845 | 7.905 | 9.721 | 1.00 | 1.71 |
| ATOM | 1761 | HD12 | LEU | 113 | 4.789 | 6.519 | 10.808 | 1.00 | 1.92 |
| ATOM | 1762 | HD13 | LEU | 113 | 3.304 | 7.355 | 10.366 | 1.00 | 1.86 |
| ATOM | 1763 | CD2 | LEU | 113 | 5.487 | 5.740 | 8.249 | 1.00 | 1.24 |
| ATOM | 1764 | HD21 | LEU | 113 | 5.868 | 4.867 | 8.757 | 1.00 | 1.64 |
| ATOM | 1765 | HD22 | LEU | 113 | 6.166 | 6.566 | 8.396 | 1.00 | 1.80 |
| ATOM | 1766 | HD23 | LEU | 113 | 5.401 | 5.531 | 7.193 | 1.00 | 1.86 |
| ATOM | 1767 | C | LEU | 113 | 1.302 | 5.379 | 7.993 | 1.00 | 0.42 |
| ATOM | 1768 | O | LEU | 113 | 0.782 | 6.460 | 7.801 | 1.00 | 0.44 |
| ATOM | 1769 | N | MET | 114 | 1.390 | 4.497 | 7.038 | 1.00 | 0.42 |
| ATOM | 1770 | HN | MET | 114 | 1.814 | 3.630 | 7.205 | 1.00 | 0.44 |
| ATOM | 1771 | CA | MET | 114 | 0.847 | 4.820 | 5.692 | 1.00 | 0.44 |
| ATOM | 1772 | HA | MET | 114 | 1.306 | 5.729 | 5.332 | 1.00 | 0.50 |
| ATOM | 1773 | CB | MET | 114 | 1.161 | 3.676 | 4.722 | 1.00 | 0.48 |
| ATOM | 1774 | HB1 | MET | 114 | 2.228 | 3.512 | 4.691 | 1.00 | 0.64 |
| ATOM | 1775 | HB2 | MET | 114 | 0.808 | 3.935 | 3.734 | 1.00 | 0.55 |
| ATOM | 1776 | CG | MET | 114 | 0.468 | 2.400 | 5.192 | 1.00 | 0.41 |
| ATOM | 1777 | HG1 | MET | 114 | −0.487 | 2.307 | 4.697 | 1.00 | 0.53 |
| ATOM | 1778 | HG2 | MET | 114 | 0.316 | 2.451 | 6.256 | 1.00 | 0.70 |
| ATOM | 1779 | SD | MET | 114 | 1.500 | 0.965 | 4.795 | 1.00 | 0.90 |
| ATOM | 1780 | CE | MET | 114 | 1.183 | 0.924 | 3.014 | 1.00 | 0.53 |
| ATOM | 1781 | HE1 | MET | 114 | 0.647 | 1.815 | 2.721 | 1.00 | 1.23 |
| ATOM | 1782 | HE2 | MET | 114 | 0.592 | 0.059 | 2.773 | 1.00 | 1.18 |
| ATOM | 1783 | HE3 | MET | 114 | 2.124 | 0.871 | 2.484 | 1.00 | 1.20 |
| ATOM | 1784 | C | MET | 114 | −0.664 | 5.028 | 5.798 | 1.00 | 0.41 |
| ATOM | 1785 | O | MET | 114 | −1.228 | 5.884 | 5.149 | 1.00 | 0.46 |
| ATOM | 1786 | N | GLU | 115 | −1.325 | 4.254 | 6.615 | 1.00 | 0.38 |
| ATOM | 1787 | HN | GLU | 115 | −0.854 | 3.569 | 7.135 | 1.00 | 0.37 |
| ATOM | 1788 | CA | GLU | 115 | −2.799 | 4.417 | 6.757 | 1.00 | 0.43 |
| ATOM | 1789 | HA | GLU | 115 | −3.252 | 4.361 | 5.780 | 1.00 | 0.46 |
| ATOM | 1790 | CB | GLU | 115 | −3.367 | 3.300 | 7.635 | 1.00 | 0.47 |
| ATOM | 1791 | HB1 | GLU | 115 | −2.950 | 3.371 | 8.627 | 1.00 | 0.88 |
| ATOM | 1792 | HB2 | GLU | 115 | −3.119 | 2.342 | 7.205 | 1.00 | 0.93 |
| ATOM | 1793 | CG | GLU | 115 | −4.889 | 3.443 | 7.711 | 1.00 | 0.84 |
| ATOM | 1794 | HG1 | GLU | 115 | −5.295 | 3.505 | 6.712 | 1.00 | 1.29 |
| ATOM | 1795 | HG2 | GLU | 115 | −5.138 | 4.341 | 8.257 | 1.00 | 1.25 |
| ATOM | 1796 | CD | GLU | 115 | −5.483 | 2.229 | 8.426 | 1.00 | 0.86 |
| ATOM | 1797 | OE1 | GLU | 115 | −4.716 | 1.370 | 8.829 | 1.00 | 1.35 |
| ATOM | 1798 | OE2 | GLU | 115 | −6.695 | 2.174 | 8.551 | 1.00 | 1.34 |
| ATOM | 1799 | C | GLU | 115 | −3.123 | 5.777 | 7.390 | 1.00 | 0.49 |
| ATOM | 1800 | O | GLU | 115 | −4.069 | 6.436 | 7.016 | 1.00 | 0.60 |
| ATOM | 1801 | N | LYS | 116 | −2.365 | 6.202 | 8.359 | 1.00 | 0.48 |
| ATOM | 1802 | HN | LYS | 116 | −1.609 | 5.661 | 8.671 | 1.00 | 0.45 |
| ATOM | 1803 | CA | LYS | 116 | −2.668 | 7.513 | 9.001 | 1.00 | 0.58 |
| ATOM | 1804 | HA | LYS | 116 | −3.739 | 7.614 | 9.108 | 1.00 | 0.66 |
| ATOM | 1805 | CB | LYS | 116 | −2.021 | 7.571 | 10.386 | 1.00 | 0.65 |
| ATOM | 1806 | HB1 | LYS | 116 | −2.103 | 8.573 | 10.781 | 1.00 | 0.73 |
| ATOM | 1807 | HB2 | LYS | 116 | −0.978 | 7.298 | 10.308 | 1.00 | 0.62 |
| ATOM | 1808 | CG | LYS | 116 | −2.739 | 6.596 | 11.321 | 1.00 | 0.72 |
| ATOM | 1809 | HG1 | LYS | 116 | −2.662 | 5.595 | 10.928 | 1.00 | 1.06 |
| ATOM | 1810 | HG2 | LYS | 116 | −3.779 | 6.877 | 11.391 | 1.00 | 1.02 |
| ATOM | 1811 | CD | LYS | 116 | −2.096 | 6.649 | 12.711 | 1.00 | 1.27 |
| ATOM | 1812 | HD1 | LYS | 116 | −2.210 | 7.642 | 13.120 | 1.00 | 1.84 |
| ATOM | 1813 | HD2 | LYS | 116 | −1.045 | 6.414 | 12.629 | 1.00 | 1.69 |
| ATOM | 1814 | CE | LYS | 116 | −2.775 | 5.637 | 13.643 | 1.00 | 1.56 |
| ATOM | 1815 | HE1 | LYS | 116 | −2.724 | 5.998 | 14.660 | 1.00 | 2.10 |
| ATOM | 1816 | HE2 | LYS | 116 | −2.264 | 4.689 | 13.574 | 1.00 | 1.73 |
| ATOM | 1817 | NZ | LYS | 116 | −4.202 | 5.459 | 13.252 | 1.00 | 2.20 |
| ATOM | 1818 | HZ1 | LYS | 116 | −4.268 | 4.755 | 12.489 | 1.00 | 2.55 |
| ATOM | 1819 | HZ2 | LYS | 116 | −4.587 | 6.367 | 12.921 | 1.00 | 2.66 |
| ATOM | 1820 | HZ3 | LYS | 116 | −4.748 | 5.129 | 14.072 | 1.00 | 2.60 |
| ATOM | 1821 | C | LYS | 116 | −2.141 | 8.665 | 8.139 | 1.00 | 0.59 |
| ATOM | 1822 | O | LYS | 116 | −2.447 | 9.815 | 8.391 | 1.00 | 0.74 |
| ATOM | 1823 | N | ASP | 117 | −1.347 | 8.373 | 7.135 | 1.00 | 0.56 |
| ATOM | 1824 | HN | ASP | 117 | −1.108 | 7.439 | 6.957 | 1.00 | 0.57 |
| ATOM | 1825 | CA | ASP | 117 | −0.794 | 9.464 | 6.268 | 1.00 | 0.63 |
| ATOM | 1826 | HA | ASP | 117 | −1.212 | 10.412 | 6.564 | 1.00 | 0.74 |
| ATOM | 1827 | CB | ASP | 117 | 0.727 | 9.518 | 6.428 | 1.00 | 0.74 |
| ATOM | 1828 | HB1 | ASP | 117 | 1.182 | 9.719 | 5.470 | 1.00 | 1.35 |
| ATOM | 1829 | HB2 | ASP | 117 | 1.083 | 8.570 | 6.804 | 1.00 | 1.02 |
| ATOM | 1830 | CG | ASP | 117 | 1.103 | 10.629 | 7.410 | 1.00 | 1.44 |
| ATOM | 1831 | OD1 | ASP | 117 | 2.187 | 11.172 | 7.271 | 1.00 | 2.14 |
| ATOM | 1832 | OD2 | ASP | 117 | 0.304 | 10.917 | 8.286 | 1.00 | 2.15 |
| ATOM | 1833 | C | ASP | 117 | −1.125 | 9.214 | 4.795 | 1.00 | 0.55 |
| ATOM | 1834 | O | ASP | 117 | −1.718 | 10.045 | 4.136 | 1.00 | 0.67 |
| ATOM | 1835 | N | SER | 118 | −0.719 | 8.096 | 4.258 | 1.00 | 0.45 |
| ATOM | 1836 | HN | SER | 118 | −0.219 | 7.445 | 4.794 | 1.00 | 0.48 |
| ATOM | 1837 | CA | SER | 118 | −0.987 | 7.832 | 2.816 | 1.00 | 0.42 |
| ATOM | 1838 | HA | SER | 118 | −0.515 | 8.600 | 2.221 | 1.00 | 0.45 |
| ATOM | 1839 | CB | SER | 118 | −0.410 | 6.472 | 2.424 | 1.00 | 0.46 |
| ATOM | 1840 | HB1 | SER | 118 | 0.552 | 6.339 | 2.900 | 1.00 | 0.53 |
| ATOM | 1841 | HB2 | SER | 118 | −0.287 | 6.429 | 1.355 | 1.00 | 0.47 |
| ATOM | 1842 | OG | SER | 118 | −1.303 | 5.445 | 2.834 | 1.00 | 0.47 |
| ATOM | 1843 | HG | SER | 118 | −1.809 | 5.168 | 2.067 | 1.00 | 0.87 |
| ATOM | 1844 | C | SER | 118 | −2.489 | 7.836 | 2.540 | 1.00 | 0.39 |
| ATOM | 1845 | O | SER | 118 | −2.950 | 8.491 | 1.634 | 1.00 | 0.41 |
| ATOM | 1846 | N | TYR | 119 | −3.257 | 7.113 | 3.309 | 1.00 | 0.40 |
| ATOM | 1847 | HN | TYR | 119 | −2.865 | 6.587 | 4.037 | 1.00 | 0.43 |
| ATOM | 1848 | CA | TYR | 119 | −4.730 | 7.075 | 3.069 | 1.00 | 0.41 |
| ATOM | 1849 | HA | TYR | 119 | −4.924 | 6.667 | 2.090 | 1.00 | 0.41 |
| ATOM | 1850 | CB | TYR | 119 | −5.388 | 6.195 | 4.123 | 1.00 | 0.46 |
| ATOM | 1851 | HB1 | TYR | 119 | −6.210 | 6.728 | 4.576 | 1.00 | 0.51 |
| ATOM | 1852 | HB2 | TYR | 119 | −4.663 | 5.949 | 4.874 | 1.00 | 0.53 |
| ATOM | 1853 | CG | TYR | 119 | −5.892 | 4.929 | 3.487 | 1.00 | 0.43 |
| ATOM | 1854 | CD1 | TYR | 119 | −5.050 | 4.165 | 2.669 | 1.00 | 0.61 |
| ATOM | 1855 | HD1 | TYR | 119 | −4.032 | 4.481 | 2.497 | 1.00 | 0.83 |
| ATOM | 1856 | CD2 | TYR | 119 | −7.207 | 4.521 | 3.711 | 1.00 | 0.46 |
| ATOM | 1857 | HD2 | TYR | 119 | −7.854 | 5.113 | 4.343 | 1.00 | 0.65 |
| ATOM | 1858 | CE1 | TYR | 119 | −5.529 | 2.994 | 2.075 | 1.00 | 0.63 |
| ATOM | 1859 | HE1 | TYR | 119 | −4.883 | 2.405 | 1.445 | 1.00 | 0.87 |
| ATOM | 1860 | CE2 | TYR | 119 | −7.683 | 3.349 | 3.118 | 1.00 | 0.45 |
| ATOM | 1861 | HE2 | TYR | 119 | −8.696 | 3.031 | 3.289 | 1.00 | 0.61 |
| ATOM | 1862 | CZ | TYR | 119 | −6.845 | 2.586 | 2.300 | 1.00 | 0.45 |
| ATOM | 1863 | OH | TYR | 119 | −7.318 | 1.434 | 1.711 | 1.00 | 0.50 |
| ATOM | 1864 | HH | TYR | 119 | −7.406 | 1.597 | 0.769 | 1.00 | 0.86 |
| ATOM | 1865 | C | TYR | 119 | −5.316 | 8.478 | 3.150 | 1.00 | 0.41 |
| ATOM | 1866 | O | TYR | 119 | −6.093 | 8.885 | 2.309 | 1.00 | 0.43 |
| ATOM | 1867 | N | ARG | 120 | −4.951 | 9.228 | 4.143 | 1.00 | 0.43 |
| ATOM | 1868 | HN | ARG | 120 | −4.324 | 8.885 | 4.813 | 1.00 | 0.43 |
| ATOM | 1869 | CA | ARG | 120 | −5.494 | 10.606 | 4.252 | 1.00 | 0.47 |
| ATOM | 1870 | HA | ARG | 120 | −6.569 | 10.582 | 4.338 | 1.00 | 0.51 |
| ATOM | 1871 | CB | ARG | 120 | −4.874 | 11.295 | 5.474 | 1.00 | 0.52 |
| ATOM | 1872 | HB1 | ARG | 120 | −5.257 | 12.301 | 5.549 | 1.00 | 0.58 |
| ATOM | 1873 | HB2 | ARG | 120 | −3.800 | 11.330 | 5.356 | 1.00 | 0.50 |
| ATOM | 1874 | CG | ARG | 120 | −5.218 | 10.524 | 6.754 | 1.00 | 0.60 |
| ATOM | 1875 | HG1 | ARG | 120 | −4.491 | 10.758 | 7.518 | 1.00 | 1.00 |
| ATOM | 1876 | HG2 | ARG | 120 | −5.192 | 9.464 | 6.550 | 1.00 | 1.23 |
| ATOM | 1877 | CD | ARG | 120 | −6.614 | 10.911 | 7.250 | 1.00 | 1.18 |
| ATOM | 1878 | HD1 | ARG | 120 | −7.354 | 10.608 | 6.528 | 1.00 | 1.78 |
| ATOM | 1879 | HD2 | ARG | 120 | −6.661 | 11.983 | 7.392 | 1.00 | 1.85 |
| ATOM | 1880 | NE | ARG | 120 | −6.886 | 10.223 | 8.543 | 1.00 | 1.72 |
| ATOM | 1881 | HE | ARG | 120 | −6.355 | 9.442 | 8.804 | 1.00 | 2.17 |
| ATOM | 1882 | CZ | ARG | 120 | −7.838 | 10.654 | 9.325 | 1.00 | 2.45 |
| ATOM | 1883 | NH1 | ARG | 120 | −8.089 | 10.036 | 10.447 | 1.00 | 3.29 |
| ATOM | 1884 | HH11 | ARG | 120 | −7.552 | 9.234 | 10.707 | 1.00 | 3.50 |
| ATOM | 1885 | HH12 | ARG | 120 | −8.818 | 10.366 | 11.048 | 1.00 | 3.98 |
| ATOM | 1886 | NH2 | ARG | 120 | −8.532 | 11.708 | 8.991 | 1.00 | 2.92 |
| ATOM | 1887 | HH21 | ARG | 120 | −8.334 | 12.186 | 8.136 | 1.00 | 2.75 |
| ATOM | 1888 | HH22 | ARG | 120 | −9.260 | 12.037 | 9.592 | 1.00 | 3.77 |
| ATOM | 1889 | C | ARG | 120 | −5.079 | 11.366 | 2.992 | 1.00 | 0.46 |
| ATOM | 1890 | O | ARG | 120 | −5.863 | 12.066 | 2.375 | 1.00 | 0.49 |
| ATOM | 1891 | N | ARG | 121 | −3.844 | 11.218 | 2.603 | 1.00 | 0.44 |
| ATOM | 1892 | HN | ARG | 121 | −3.239 | 10.641 | 3.115 | 1.00 | 0.43 |
| ATOM | 1893 | CA | ARG | 121 | −3.347 | 11.910 | 1.386 | 1.00 | 0.45 |
| ATOM | 1894 | HA | ARG | 121 | −3.618 | 12.955 | 1.429 | 1.00 | 0.49 |
| ATOM | 1895 | CB | ARG | 121 | −1.825 | 11.783 | 1.313 | 1.00 | 0.47 |
| ATOM | 1896 | HB1 | ARG | 121 | −1.470 | 12.338 | 0.460 | 1.00 | 0.49 |
| ATOM | 1897 | HB2 | ARG | 121 | −1.559 | 10.741 | 1.202 | 1.00 | 0.48 |
| ATOM | 1898 | CG | ARG | 121 | −1.185 | 12.341 | 2.594 | 1.00 | 0.56 |
| ATOM | 1899 | HG1 | ARG | 121 | −0.442 | 11.645 | 2.952 | 1.00 | 1.20 |
| ATOM | 1900 | HG2 | ARG | 121 | −1.947 | 12.468 | 3.349 | 1.00 | 1.08 |
| ATOM | 1901 | CD | ARG | 121 | −0.518 | 13.695 | 2.321 | 1.00 | 1.18 |
| ATOM | 1902 | HD1 | ARG | 121 | −1.275 | 14.440 | 2.130 | 1.00 | 1.73 |
| ATOM | 1903 | HD2 | ARG | 121 | 0.136 | 13.615 | 1.465 | 1.00 | 1.91 |
| ATOM | 1904 | NE | ARG | 121 | 0.284 | 14.091 | 3.513 | 1.00 | 1.81 |
| ATOM | 1905 | HE | ARG | 121 | 0.159 | 13.622 | 4.364 | 1.00 | 2.29 |
| ATOM | 1906 | CZ | ARG | 121 | 1.164 | 15.050 | 3.420 | 1.00 | 2.47 |
| ATOM | 1907 | NH1 | ARG | 121 | 1.886 | 15.375 | 4.458 | 1.00 | 3.36 |
| ATOM | 1908 | HH11 | ARG | 121 | 1.765 | 14.889 | 5.324 | 1.00 | 3.67 |
| ATOM | 1909 | HH12 | ARG | 121 | 2.561 | 16.110 | 4.387 | 1.00 | 3.97 |
| ATOM | 1910 | NH2 | ARG | 121 | 1.321 | 15.686 | 2.292 | 1.00 | 2.78 |
| ATOM | 1911 | HH21 | ARG | 121 | 0.766 | 15.438 | 1.497 | 1.00 | 2.59 |
| ATOM | 1912 | HH22 | ARG | 121 | 1.995 | 16.421 | 2.222 | 1.00 | 3.55 |
| ATOM | 1913 | C | ARG | 121 | −3.959 | 11.273 | 0.131 | 1.00 | 0.42 |
| ATOM | 1914 | O | ARG | 121 | −4.306 | 11.957 | −0.806 | 1.00 | 0.44 |
| ATOM | 1915 | N | PHE | 122 | −4.079 | 9.967 | 0.099 | 1.00 | 0.39 |
| ATOM | 1916 | HN | PHE | 122 | −3.782 | 9.429 | 0.861 | 1.00 | 0.40 |
| ATOM | 1917 | CA | PHE | 122 | −4.647 | 9.296 | −1.114 | 1.00 | 0.40 |
| ATOM | 1918 | HA | PHE | 122 | −4.008 | 9.489 | −1.963 | 1.00 | 0.42 |
| ATOM | 1919 | CB | PHE | 122 | −4.746 | 7.783 | −0.886 | 1.00 | 0.40 |
| ATOM | 1920 | HB1 | PHE | 122 | −5.785 | 7.498 | −0.826 | 1.00 | 0.54 |
| ATOM | 1921 | HB2 | PHE | 122 | −4.253 | 7.523 | 0.032 | 1.00 | 0.48 |
| ATOM | 1922 | CG | PHE | 122 | −4.092 | 7.041 | −2.028 | 1.00 | 0.38 |
| ATOM | 1923 | CD1 | PHE | 122 | −2.709 | 6.825 | −2.025 | 1.00 | 0.49 |
| ATOM | 1924 | HD1 | PHE | 122 | −2.106 | 7.196 | −1.210 | 1.00 | 0.67 |
| ATOM | 1925 | CD2 | PHE | 122 | −4.874 | 6.559 | −3.085 | 1.00 | 0.69 |
| ATOM | 1926 | HD2 | PHE | 122 | −5.941 | 6.726 | −3.087 | 1.00 | 0.92 |
| ATOM | 1927 | CE1 | PHE | 122 | −2.108 | 6.126 | −3.079 | 1.00 | 0.68 |
| ATOM | 1928 | HE1 | PHE | 122 | −1.041 | 5.959 | −3.077 | 1.00 | 0.90 |
| ATOM | 1929 | CE2 | PHE | 122 | −4.272 | 5.863 | −4.140 | 1.00 | 0.85 |
| ATOM | 1930 | HE2 | PHE | 122 | −4.876 | 5.492 | −4.955 | 1.00 | 1.15 |
| ATOM | 1931 | CZ | PHE | 122 | −2.889 | 5.646 | −4.137 | 1.00 | 0.78 |
| ATOM | 1932 | HZ | PHE | 122 | −2.426 | 5.107 | −4.950 | 1.00 | 0.98 |
| ATOM | 1933 | C | PHE | 122 | −6.043 | 9.836 | −1.397 | 1.00 | 0.41 |
| ATOM | 1934 | O | PHE | 122 | −6.356 | 10.202 | −2.507 | 1.00 | 0.47 |
| ATOM | 1935 | N | LEU | 123 | −6.886 | 9.892 | −0.410 | 1.00 | 0.39 |
| ATOM | 1936 | HN | LEU | 123 | −6.620 | 9.593 | 0.484 | 1.00 | 0.38 |
| ATOM | 1937 | CA | LEU | 123 | −8.258 | 10.411 | −0.648 | 1.00 | 0.43 |
| ATOM | 1938 | HA | LEU | 123 | −8.748 | 9.803 | −1.394 | 1.00 | 0.46 |
| ATOM | 1939 | CB | LEU | 123 | −9.052 | 10.359 | 0.659 | 1.00 | 0.45 |
| ATOM | 1940 | HB1 | LEU | 123 | −10.028 | 10.794 | 0.509 | 1.00 | 0.49 |
| ATOM | 1941 | HB2 | LEU | 123 | −8.524 | 10.916 | 1.420 | 1.00 | 0.45 |
| ATOM | 1942 | CG | LEU | 123 | −9.200 | 8.897 | 1.104 | 1.00 | 0.45 |
| ATOM | 1943 | HG | LEU | 123 | −8.224 | 8.433 | 1.124 | 1.00 | 0.42 |
| ATOM | 1944 | CD1 | LEU | 123 | −9.809 | 8.845 | 2.505 | 1.00 | 0.50 |
| ATOM | 1945 | HD11 | LEU | 123 | −9.851 | 7.818 | 2.840 | 1.00 | 1.17 |
| ATOM | 1946 | HD12 | LEU | 123 | −10.807 | 9.256 | 2.480 | 1.00 | 1.13 |
| ATOM | 1947 | HD13 | LEU | 123 | −9.199 | 9.420 | 3.185 | 1.00 | 1.05 |
| ATOM | 1948 | CD2 | LEU | 123 | −10.106 | 8.131 | 0.130 | 1.00 | 0.49 |
| ATOM | 1949 | HD21 | LEU | 123 | −10.854 | 8.796 | −0.276 | 1.00 | 1.11 |
| ATOM | 1950 | HD22 | LEU | 123 | −10.593 | 7.324 | 0.656 | 1.00 | 1.18 |
| ATOM | 1951 | HD23 | LEU | 123 | −9.510 | 7.725 | −0.674 | 1.00 | 1.11 |
| ATOM | 1952 | C | LEU | 123 | −8.158 | 11.851 | −1.156 | 1.00 | 0.45 |
| ATOM | 1953 | O | LEU | 123 | −8.878 | 12.256 | −2.046 | 1.00 | 0.54 |
| ATOM | 1954 | N | LYS | 124 | −7.262 | 12.625 | −0.605 | 1.00 | 0.43 |
| ATOM | 1955 | HN | LYS | 124 | −6.683 | 12.277 | 0.110 | 1.00 | 0.41 |
| ATOM | 1956 | CA | LYS | 124 | −7.109 | 14.035 | −1.069 | 1.00 | 0.48 |
| ATOM | 1957 | HA | LYS | 124 | −8.060 | 14.386 | −1.441 | 1.00 | 0.53 |
| ATOM | 1958 | CB | LYS | 124 | −6.675 | 14.918 | 0.102 | 1.00 | 0.55 |
| ATOM | 1959 | HB1 | LYS | 124 | −6.390 | 15.892 | −0.267 | 1.00 | 0.60 |
| ATOM | 1960 | HB2 | LYS | 124 | −5.834 | 14.462 | 0.603 | 1.00 | 0.55 |
| ATOM | 1961 | CG | LYS | 124 | −7.836 | 15.068 | 1.086 | 1.00 | 0.64 |
| ATOM | 1962 | HG1 | LYS | 124 | −8.123 | 14.096 | 1.457 | 1.00 | 0.81 |
| ATOM | 1963 | HG2 | LYS | 124 | −8.677 | 15.524 | 0.581 | 1.00 | 1.05 |
| ATOM | 1964 | CD | LYS | 124 | −7.401 | 15.953 | 2.257 | 1.00 | 1.08 |
| ATOM | 1965 | HD1 | LYS | 124 | −7.103 | 16.921 | 1.885 | 1.00 | 1.62 |
| ATOM | 1966 | HD2 | LYS | 124 | −6.566 | 15.490 | 2.764 | 1.00 | 1.52 |
| ATOM | 1967 | CD | LYS | 124 | −8.564 | 16.120 | 3.238 | 1.00 | 1.26 |
| ATOM | 1968 | HE1 | LYS | 124 | −9.495 | 15.905 | 2.734 | 1.00 | 1.66 |
| ATOM | 1969 | HE2 | LYS | 124 | −8.581 | 17.135 | 3.604 | 1.00 | 1.84 |
| ATOM | 1970 | NZ | LYS | 124 | −8.393 | 15.184 | 4.384 | 1.00 | 1.82 |
| ATOM | 1971 | HZ1 | LYS | 124 | −9.095 | 15.405 | 5.119 | 1.00 | 2.26 |
| ATOM | 1972 | HZ2 | LYS | 124 | −7.435 | 15.287 | 4.776 | 1.00 | 2.35 |
| ATOM | 1973 | HZ3 | LYS | 124 | −8.533 | 14.207 | 4.058 | 1.00 | 2.23 |
| ATOM | 1974 | C | LYS | 124 | −6.069 | 14.124 | −2.195 | 1.00 | 0.47 |
| ATOM | 1975 | O | LYS | 124 | −5.784 | 15.194 | −2.695 | 1.00 | 0.52 |
| ATOM | 1976 | N | SER | 125 | −5.491 | 13.022 | −2.596 | 1.00 | 0.46 |
| ATOM | 1977 | HN | SER | 125 | −5.724 | 12.165 | −2.182 | 1.00 | 0.49 |
| ATOM | 1978 | CA | SER | 125 | −4.466 | 13.076 | −3.684 | 1.00 | 0.49 |
| ATOM | 1979 | HA | SER | 125 | −3.780 | 13.886 | −3.486 | 1.00 | 0.54 |
| ATOM | 1980 | CB | SER | 125 | −3.689 | 11.759 | −3.739 | 1.00 | 0.55 |
| ATOM | 1981 | HB1 | SER | 125 | −3.181 | 11.598 | −2.800 | 1.00 | 1.13 |
| ATOM | 1982 | HB2 | SER | 125 | −2.961 | 11.804 | −4.531 | 1.00 | 1.15 |
| ATOM | 1983 | OG | SER | 125 | −4.591 | 10.691 | −3.997 | 1.00 | 1.34 |
| ATOM | 1984 | HG | SER | 125 | −4.433 | 10.380 | −4.892 | 1.00 | 1.80 |
| ATOM | 1985 | C | SER | 125 | −5.144 | 13.310 | −5.035 | 1.00 | 0.46 |
| ATOM | 1986 | O | SER | 125 | −6.282 | 12.941 | −5.246 | 1.00 | 0.47 |
| ATOM | 1987 | N | ARG | 126 | −4.443 | 13.914 | −5.955 | 1.00 | 0.52 |
| ATOM | 1988 | HN | ARG | 126 | −3.525 | 14.196 | −5.761 | 1.00 | 0.56 |
| ATOM | 1989 | CA | ARG | 126 | −5.027 | 14.172 | −7.302 | 1.00 | 0.58 |
| ATOM | 1990 | HA | ARG | 126 | −5.954 | 14.715 | −7.189 | 1.00 | 0.58 |
| ATOM | 1991 | CB | ARG | 126 | −4.052 | 15.009 | −8.132 | 1.00 | 0.71 |
| ATOM | 1992 | HB1 | ARG | 126 | −4.391 | 15.050 | −9.156 | 1.00 | 1.12 |
| ATOM | 1993 | HB2 | ARG | 126 | −3.069 | 14.560 | −8.095 | 1.00 | 1.28 |
| ATOM | 1994 | CG | ARG | 126 | −3.989 | 16.427 | −7.561 | 1.00 | 1.29 |
| ATOM | 1995 | HG1 | ARG | 126 | −3.651 | 16.388 | −6.536 | 1.00 | 1.89 |
| ATOM | 1996 | HG2 | ARG | 126 | −4.973 | 16.872 | −7.598 | 1.00 | 1.94 |
| ATOM | 1997 | CD | ARG | 126 | −3.016 | 17.274 | −8.384 | 1.00 | 1.86 |
| ATOM | 1998 | HD1 | ARG | 126 | −3.016 | 18.288 | −8.009 | 1.00 | 2.13 |
| ATOM | 1999 | HD2 | ARG | 126 | −3.324 | 17.274 | −9.418 | 1.00 | 2.26 |
| ATOM | 2000 | NE | ARG | 126 | −1.644 | 16.705 | −8.277 | 1.00 | 2.80 |
| ATOM | 2001 | HE | ARG | 126 | −1.438 | 16.050 | −7.577 | 1.00 | 3.15 |
| ATOM | 2002 | CZ | ARG | 126 | −0.715 | 17.081 | −9.112 | 1.00 | 3.67 |
| ATOM | 2003 | NH1 | ARG | 126 | 0.490 | 16.589 | −9.016 | 1.00 | 4.70 |
| ATOM | 2004 | HH11 | ARC | 126 | 0.702 | 15.922 | −8.301 | 1.00 | 4.91 |
| ATOM | 2005 | HH12 | ARG | 126 | 1.201 | 16.878 | −9.657 | 1.00 | 5.46 |
| ATOM | 2006 | NH2 | ARG | 126 | −0.991 | 17.952 | −10.045 | 1.00 | 3.92 |
| ATOM | 2007 | HH21 | ARG | 126 | −1.914 | 18.330 | −10.118 | 1.00 | 3.48 |
| ATOM | 2008 | HH22 | ARG | 126 | −0.280 | 18.241 | −10.685 | 1.00 | 4.82 |
| ATOM | 2009 | C | ARG | 126 | −5.305 | 12.846 | −8.015 | 1.00 | 0.58 |
| ATOM | 2010 | O | ARG | 126 | −6.275 | 12.714 | −8.734 | 1.00 | 0.60 |
| ATOM | 2011 | N | PHE | 127 | −4.460 | 11.863 | −7.838 | 1.00 | 0.60 |
| ATOM | 2012 | HN | PHE | 127 | −3.675 | 11.984 | −7.264 | 1.00 | 0.61 |
| ATOM | 2013 | CA | PHE | 127 | −4.693 | 10.563 | −8.531 | 1.00 | 0.65 |
| ATOM | 2014 | HA | PHE | 127 | −4.690 | 10.713 | −9.600 | 1.00 | 0.72 |
| ATOM | 2015 | CB | PHE | 127 | −3.592 | 9.570 | −8.147 | 1.00 | 0.73 |
| ATOM | 2016 | HB1 | PHE | 127 | −3.856 | 8.587 | −8.507 | 1.00 | 0.80 |
| ATOM | 2017 | HB2 | PHE | 127 | −3.495 | 9.543 | −7.071 | 1.00 | 0.69 |
| ATOM | 2018 | CG | PHE | 127 | −2.276 | 9.989 | −8.755 | 1.00 | 0.84 |
| ATOM | 2019 | CD1 | PHE | 127 | −1.398 | 10.804 | −8.031 | 1.00 | 0.85 |
| ATOM | 2020 | HD1 | PHE | 127 | −1.665 | 11.137 | −7.038 | 1.00 | 0.82 |
| ATOM | 2021 | CD2 | PHE | 127 | −1.930 | 9.557 | −10.041 | 1.00 | 0.98 |
| ATOM | 2022 | HD2 | PHE | 127 | −2.607 | 8.929 | −10.601 | 1.00 | 1.03 |
| ATOM | 2023 | CE1 | PHE | 127 | −0.174 | 11.187 | −8.592 | 1.00 | 0.98 |
| ATOM | 2024 | HE1 | PHE | 127 | 0.503 | 11.815 | −8.032 | 1.00 | 1.03 |
| ATOM | 2025 | CE2 | PHE | 127 | −0.706 | 9.941 | −10.603 | 1.00 | 1.10 |
| ATOM | 2026 | HE2 | PHE | 127 | −0.439 | 9.609 | −11.595 | 1.00 | 1.23 |
| ATOM | 2027 | CZ | PHE | 127 | 0.172 | 10.756 | −9.878 | 1.00 | 1.09 |
| ATOM | 2028 | HZ | PHE | 127 | 1.116 | 11.051 | −10.310 | 1.00 | 1.19 |
| ATOM | 2029 | C | PHE | 127 | −6.041 | 9.987 | −8.093 | 1.00 | 0.58 |
| ATOM | 2030 | O | PHE | 127 | −6.859 | 9.611 | −8.907 | 1.00 | 0.59 |
| ATOM | 2031 | N | TYR | 128 | −6.280 | 9.920 | −6.813 | 1.00 | 0.52 |
| ATOM | 2032 | HN | TYR | 128 | −5.609 | 10.233 | −6.171 | 1.00 | 0.54 |
| ATOM | 2033 | CA | TYR | 128 | −7.578 | 9.373 | −6.327 | 1.00 | 0.48 |
| ATOM | 2034 | HA | TYR | 128 | −7.701 | 8.362 | −6.679 | 1.00 | 0.51 |
| ATOM | 2035 | CB | TYR | 128 | −7.602 | 9.392 | −4.801 | 1.00 | 0.48 |
| ATOM | 2036 | HB1 | TYR | 128 | −7.542 | 10.415 | −4.463 | 1.00 | 0.48 |
| ATOM | 2037 | HB2 | TYR | 128 | −6.762 | 8.832 | −4.417 | 1.00 | 0.51 |
| ATOM | 2038 | CG | TYR | 128 | −8.892 | 8.779 | −4.309 | 1.00 | 0.48 |
| ATOM | 2039 | CD1 | TYR | 128 | −8.961 | 7.403 | −4.059 | 1.00 | 0.51 |
| ATOM | 2040 | HD1 | TYR | 128 | −8.093 | 6.781 | −4.220 | 1.00 | 0.53 |
| ATOM | 2041 | CD2 | TYR | 128 | −10.017 | 9.586 | −4.100 | 1.00 | 0.51 |
| ATOM | 2042 | HD2 | TYR | 128 | −9.964 | 10.647 | −4.292 | 1.00 | 0.52 |
| ATOM | 2043 | CE1 | TYR | 128 | −10.155 | 6.834 | −3.601 | 1.00 | 0.56 |
| ATOM | 2044 | HE1 | TYR | 128 | −10.208 | 5.772 | −3.408 | 1.00 | 0.61 |
| ATOM | 2045 | CE2 | TYR | 128 | −11.211 | 9.016 | −3.642 | 1.00 | 0.55 |
| ATOM | 2046 | HE2 | TYR | 128 | −12.079 | 9.638 | −3.482 | 1.00 | 0.61 |
| ATOM | 2047 | CZ | TYR | 128 | −11.280 | 7.640 | −3.393 | 1.00 | 0.58 |
| ATOM | 2048 | OH | TYR | 128 | −12.458 | 7.079 | −2.942 | 1.00 | 0.66 |
| ATOM | 2049 | HH | TYR | 128 | −12.323 | 6.797 | −2.034 | 1.00 | 1.10 |
| ATOM | 2050 | C | TYR | 128 | −8.721 | 10.241 | −6.847 | 1.00 | 0.45 |
| ATOM | 2051 | O | TYR | 128 | −9.718 | 9.751 | −7.336 | 1.00 | 0.47 |
| ATOM | 2052 | N | LEU | 129 | −8.581 | 11.531 | −6.724 | 1.00 | 0.45 |
| ATOM | 2053 | HN | LEU | 129 | −7.770 | 11.894 | −6.312 | 1.00 | 0.46 |
| ATOM | 2054 | CA | LEU | 129 | −9.656 | 12.453 | −7.181 | 1.00 | 0.49 |
| ATOM | 2055 | HA | LEU | 129 | −10.566 | 12.245 | −6.638 | 1.00 | 0.49 |
| ATOM | 2056 | CB | LEU | 129 | −9.218 | 13.896 | −6.916 | 1.00 | 0.53 |
| ATOM | 2057 | HB1 | LEU | 129 | −9.912 | 14.578 | −7.383 | 1.00 | 0.60 |
| ATOM | 2058 | HB2 | LEU | 129 | −8.233 | 14.046 | −7.335 | 1.00 | 0.55 |
| ATOM | 2059 | CG | LEU | 129 | −9.177 | 14.156 | −5.402 | 1.00 | 0.50 |
| ATOM | 2060 | HG | LEU | 129 | −8.561 | 13.403 | −4.930 | 1.00 | 0.45 |
| ATOM | 2061 | CD1 | LEU | 129 | −8.578 | 15.539 | −5.127 | 1.00 | 0.59 |
| ATOM | 2062 | HD11 | LEU | 129 | −7.762 | 15.725 | −5.810 | 1.00 | 1.16 |
| ATOM | 2063 | HD12 | LEU | 129 | −8.212 | 15.576 | −4.112 | 1.00 | 1.16 |
| ATOM | 2064 | HD13 | LEU | 129 | −9.339 | 16.293 | −5.262 | 1.00 | 1.20 |
| ATOM | 2065 | CD2 | LEU | 129 | −10.593 | 14.094 | −4.814 | 1.00 | 0.55 |
| ATOM | 2066 | HD21 | LEU | 129 | −11.317 | 14.343 | −5.574 | 1.00 | 1.20 |
| ATOM | 2067 | HD22 | LEU | 129 | −10.673 | 14.796 | −3.998 | 1.00 | 1.06 |
| ATOM | 2068 | HD23 | LEU | 129 | −10.785 | 13.096 | −4.448 | 1.00 | 1.16 |
| ATOM | 2069 | C | LEU | 129 | −9.901 | 12.261 | −8.678 | 1.00 | 0.55 |
| ATOM | 2070 | O | LEU | 129 | −11.027 | 12.146 | −9.119 | 1.00 | 0.61 |
| ATOM | 2071 | N | ASP | 130 | −8.863 | 12.218 | −9.467 | 1.00 | 0.59 |
| ATOM | 2072 | HN | ASP | 130 | −7.959 | 12.306 | −9.100 | 1.00 | 0.60 |
| ATOM | 2073 | CA | ASP | 130 | −9.063 | 12.023 | −10.930 | 1.00 | 0.67 |
| ATOM | 2074 | HA | ASP | 130 | −9.667 | 12.828 | −11.323 | 1.00 | 0.75 |
| ATOM | 2075 | CB | ASP | 130 | −7.705 | 12.003 | −11.636 | 1.00 | 0.76 |
| ATOM | 2076 | HB1 | ASP | 130 | −7.838 | 11.690 | −12.661 | 1.00 | 0.81 |
| ATOM | 2077 | HB2 | ASP | 130 | −7.048 | 11.311 | −11.130 | 1.00 | 0.74 |
| ATOM | 2078 | CG | ASP | 130 | −7.091 | 13.403 | −11.609 | 1.00 | 0.87 |
| ATOM | 2079 | OD1 | ASP | 130 | −7.835 | 14.351 | −11.416 | 1.00 | 1.23 |
| ATOM | 2080 | OD2 | ASP | 130 | −5.888 | 13.504 | −11.783 | 1.00 | 1.58 |
| ATOM | 2081 | C | ASP | 130 | −9.778 | 10.693 | −11.158 | 1.00 | 0.65 |
| ATOM | 2082 | O | ASP | 130 | −10.766 | 10.615 | −11.859 | 1.00 | 0.72 |
| ATOM | 2083 | N | LEU | 131 | −9.292 | 9.645 | −10.556 | 1.00 | 0.61 |
| ATOM | 2084 | HN | LEU | 131 | −8.499 | 9.731 | −9.986 | 1.00 | 0.59 |
| ATOM | 2085 | CA | LEU | 131 | −9.949 | 8.321 | −10.719 | 1.00 | 0.66 |
| ATOM | 2086 | HA | LEU | 131 | −10.108 | 8.123 | −11.768 | 1.00 | 0.73 |
| ATOM | 2087 | CB | LEU | 131 | −9.066 | 7.221 | −10.112 | 1.00 | 0.70 |
| ATOM | 2088 | HB1 | LEU | 131 | −9.501 | 6.255 | −10.319 | 1.00 | 0.78 |
| ATOM | 2089 | HB2 | LEU | 131 | −9.011 | 7.363 | −9.042 | 1.00 | 0.65 |
| ATOM | 2090 | CG | LEU | 131 | −7.649 | 7.280 | −10.701 | 1.00 | 0.76 |
| ATOM | 2091 | HG | LEU | 131 | −7.339 | 8.308 | −10.805 | 1.00 | 0.74 |
| ATOM | 2092 | CD1 | LEU | 131 | −6.686 | 6.551 | −9.763 | 1.00 | 0.85 |
| ATOM | 2093 | HD11 | LEU | 131 | −5.747 | 6.382 | −10.270 | 1.00 | 1.47 |
| ATOM | 2094 | HD12 | LEU | 131 | −7.114 | 5.603 | −9.472 | 1.00 | 1.25 |
| ATOM | 2095 | HD13 | LEU | 131 | −6.516 | 7.154 | −8.883 | 1.00 | 1.30 |
| ATOM | 2096 | CD2 | LEU | 131 | −7.619 | 6.591 | −12.073 | 1.00 | 0.85 |
| ATOM | 2097 | HD21 | LEU | 131 | −8.623 | 6.483 | −12.452 | 1.00 | 1.38 |
| ATOM | 2098 | HD22 | LEU | 131 | −7.167 | 5.616 | −11.974 | 1.00 | 1.27 |
| ATOM | 2099 | HD23 | LEU | 131 | −7.038 | 7.187 | −12.760 | 1.00 | 1.36 |
| ATOM | 2100 | C | LEU | 131 | −11.296 | 8.339 | −9.994 | 1.00 | 0.67 |
| ATOM | 2101 | O | LEU | 131 | −12.196 | 7.593 | −10.325 | 1.00 | 0.82 |
| ATOM | 2102 | N | THR | 132 | −11.426 | 9.177 | −8.992 | 1.00 | 0.58 |
| ATOM | 2103 | HN | THR | 132 | −10.679 | 9.756 | −8.743 | 1.00 | 0.52 |
| ATOM | 2104 | CA | THR | 132 | −12.702 | 9.242 | −8.217 | 1.00 | 0.65 |
| ATOM | 2105 | HA | THR | 132 | −13.392 | 8.501 | −8.588 | 1.00 | 0.79 |
| ATOM | 2106 | CB | THR | 132 | −12.417 | 8.962 | −6.735 | 1.00 | 0.60 |
| ATOM | 2107 | HB | THR | 132 | −13.350 | 8.813 | −6.214 | 1.00 | 0.74 |
| ATOM | 2108 | OG1 | THR | 132 | −11.734 | 10.071 | −6.169 | 1.00 | 0.53 |
| ATOM | 2109 | HG1 | THR | 132 | −10.823 | 10.044 | −6.471 | 1.00 | 1.02 |
| ATOM | 2110 | CG2 | THR | 132 | −11.555 | 7.703 | −6.592 | 1.00 | 0.79 |
| ATOM | 2111 | HG21 | THR | 132 | −12.092 | 6.965 | −6.013 | 1.00 | 1.38 |
| ATOM | 2112 | HG22 | THR | 132 | −10.634 | 7.953 | −6.088 | 1.00 | 1.32 |
| ATOM | 2113 | HG23 | THR | 132 | −11.332 | 7.299 | −7.567 | 1.00 | 1.28 |
| ATOM | 2114 | C | THR | 132 | −13.339 | 10.629 | −8.348 | 1.00 | 0.77 |
| ATOM | 2115 | O | THR | 132 | −13.331 | 11.414 | −7.421 | 1.00 | 0.84 |
| ATOM | 2116 | N | ASN | 133 | −13.911 | 10.931 | −9.481 | 1.00 | 0.97 |
| ATOM | 2117 | HN | ASN | 133 | −13.917 | 10.281 | −10.213 | 1.00 | 1.04 |
| ATOM | 2118 | CA | ASN | 133 | −14.569 | 12.260 | −9.651 | 1.00 | 1.21 |
| ATOM | 2119 | HA | ASN | 133 | −14.028 | 13.005 | −9.086 | 1.00 | 1.15 |
| ATOM | 2120 | CB | ASN | 133 | −14.583 | 12.652 | −11.131 | 1.00 | 1.44 |
| ATOM | 2121 | HB1 | ASN | 133 | −15.359 | 13.383 | −11.301 | 1.00 | 1.70 |
| ATOM | 2122 | HB2 | ASN | 133 | −14.778 | 11.775 | −11.732 | 1.00 | 1.60 |
| ATOM | 2123 | CG | ASN | 133 | −13.231 | 13.247 | −11.522 | 1.00 | 1.35 |
| ATOM | 2124 | OD1 | ASN | 133 | −13.027 | 14.440 | −11.414 | 1.00 | 1.57 |
| ATOM | 2125 | ND2 | ASN | 133 | −12.292 | 12.464 | −11.972 | 1.00 | 1.89 |
| ATOM | 2126 | HD21 | ASN | 133 | −12.455 | 11.502 | −12.057 | 1.00 | 2.51 |
| ATOM | 2127 | HD22 | ASN | 133 | −11.422 | 12.839 | −12.225 | 1.00 | 1.97 |
| ATOM | 2128 | C | ASN | 133 | −16.010 | 12.179 | −9.142 | 1.00 | 1.48 |
| ATOM | 2129 | O | ASN | 133 | −16.515 | 11.115 | −8.847 | 1.00 | 1.63 |
| END | | | | | | | | | |

### REFERENCES

1. Neer, E. J. (1995) Cell 80, 249-257.

2. Sprang, S. R. (1997) Annu. Rev. Biochem 66, 639-678.

3. LeVine, H., III. (1999) Mol. Neurobiol. 19, 111-149.

4. Farfel, Z., Bourne, H. R., and Iiri, T. (1999) N. Engl J. Med. 340, 1012-1020.

5. Berghuis, A. M., Lee, E., Raw, A. S., and Gilman, A. (1996) Structure 4, 1277-1290.

6. Mixon,, M. B., Lee, E., Coleman, D. F., and Berghuis, A. (1995) Science 270, 954-60.

7. Coleman, D. E., Berghuis, A. M., Lee, E., and Linder. (1994) Science 265, 1405-12.

8. Tesmer, J. J. G., Berman, D. M., Gilman, A. G., and Sprang, S. R. (1997) Cell 89, 251-261.

9. Dohlman, H. G., and Thomer, J. (1997) J. Biol. Chem. 272, 3871-3874.

10. Kehrl, J. H. (1998) Immunity 8, 1-10.

11. Arshavsky, V. Y., and Pugh, E. N., Jr. (1998) Neuron 20, 11-14.

12. Zerangue, N., and Jan, L. Y. (1998) Curr. Biol. 8, R313-R316.

13. De Vries, L., and Farquhar, M. G. (1999) Trends Cell Biol. 9, 138-144.

14. Kozasa, T., Jiang, X., Hart, M. J., Sternweis, P. M., Singer, W. D., Gilman, A. G., Bollag, G., and Sternweis, P. C. (1998) Science 280, 2109-2112.

15. Nomoto, S., Adachi, K., Yang, L.-X., Hirata, Y., Muraguchi, S., and Kiuchi, K. (1997) Biochem. Biophys. Res. Commun. 241, 281-287.

16. Gold, S. J., Ni, Y. G., Dohlman, H. G., and Nestler, E. (1997)J. Neurosci. 17, 8024-8037.

17. Berman, D. M., Kozasa, T., and Gilman, A. G. (1996) J. Biol. Chem. 271,27209-27212.

18. Srinivasa, S. P., Watson, N., Overton, M. C., and Blumer, K. J. (1998) J. Biol. Chem. 273, 1529-1533.

19. Posner, B. A., Mukhopadhyay, S., Tesmer, J. J., Gilman, A. G., and Ross, E. M. (1999) Biochemistry 38, 7773-7779.

20. Natochin, M., McEntaffer, R. L., and Artemyev, N. O. (1998) J. Biol. Chem. 273, 6731-6735.

21. Watson, N., Linder, M. E., and Druey, K. M. (1996) Nature 383, 172-175.

22. My, F., Chanda, P. K., Cockett, M. I., Edris, W., Jones, P. G., and Powers, R. (1999) J. Biomol. NMR 15, 339-340.

23. Piotto, M., Saudek, V., and Sklenar, V. (1992) J. Biomol. NMR 2, 661-5.

24. Grzesiek, S., and Bax, A. (1993) J. Am. Chem. Soc 115, 12593-4.

25. Marion, D., Ikura, M., Tschudin, R., and Bax, A. (1989) J. Magn. Reson 85; 393-9.

26. Vuister, G. W., and Bax, A. (1993) J. Am. Chem. Soc 115, 7772-7.

27. Archer, S. J., Ikura, M., Torchia, D. A., and Bax, A. (1991) J. Magn. Reson 95, 636-41.

28. Bax, A. and Pochapsky, S. S. (1992) Journal of Magnetic Resonance 99, 638-643.

29. Powers, R., Gronenborn, A. M., Clore, G. M., and Bax, A. (1 991) J. Magn. Reson. 94, 209-13.

30. Vuister, G. W., Delaglio, F., and Bax, A. (1992) J. Am. Chem. Soc 114, 9674-5.

31. Grzesiek, S., Kuboniwa, H., Hinck, A. P., and Bax, A. (1995) J. Am. Chem. Soc 117, 5312-15.

32. Marion, D., Driscoll, P. C., Kay, L. E., Wingfield, P. T., Bax, A., Gronenborn, A. M., and Clore, G. M. (1989) Biochemistry 28, 6150-6.

33. Zuiderweg, E. R. P., and Fesik, S. W. (1989) Biochemistry 28, 2387-91.

34. Zuiderweg, E. R. P., McIntosh, L. P., Dahlquist, F. W., and Fesik, S. W. (1990) J. Magn. Reson 86,210-16.

35. Ikura, M., Kay, L. E., Tschudin, R., and Bax, A. (1990) J. Magn. Reson 86, 204-9.

36. Delaglio, F., Grzesiek, S., Vuister, G. W., Zhu, G., Pfeifer, J., and Bax, A. (1995) J. Biomol. NMR 6, 277-293.

37. Garrett, D. S., Powers, R., Gronenborn, A. M., and Clore, G. M. (1991) J. Magn. Reson. 95, 214-20.

38. Zhu, G., and Bax, A. (1992) J. Magn. Reson. 100, 202-7.

39. Williamson, M. P., Havel, T. F., and Wuethrich, K. (1985) J. Mol. Biol 182, 295-315.

40. Clore, G. M., Nilges, M., Sukumaran, D. K., Bruenger, A. T., Karplus, M., and Gronenborn, A. M. (1986) EMBO J. 2729-35.

41. Wuthrich, K., Billeter, M., and Braun, W. (1983) J. Mol. Biol. 169, 949-961.

42. Powers, R., Garrett, D. S., March, C. J., Frieden, E. A., Gronenborn, A. M., and Clore, G. M. (1993) Biochemistry 32, 6744-62.

43. Cornilescu, G., Delaglio, F., and Bax, A. (1999) J. Biomol. NMR 13, 289-302.

44. Bax, A., Max, D., and Zax, D. (1992) J. Am. Chem. Soc. 114, 6923-5.

45. Vuister, G. W., Clore, G. M., Gronenborn, A. M., Powers, R., Garrett, D. S., Tschudin, R., and Bax, A. (1993) Biochemistry 32, 6744-62.

46. Zuiderweg, E. R. P., Boelens, R., and Kaptein, R. (1985) Biopolymers 24, 601-1 1.

47. Kraulis, P. J., Clore, G. M., Nilges, M., Jones, T. A., Pettersson, G., Knowles, J., and Gronenborn, A. M. (1989) Biochemistry 28, 7241-57.

48. Nilges, M., Gronenborn, A. M., Bruenger, A. T., and Clore, G. M. (1988) Protein Eng 2,27-38

49. Clore, G. M., Appella, E., Yamada, M., Matsushima, K., and Gronenborn, A. M. (1990) Biochemistry 29, 1689-96.

50. Brunger, A. T. (1993)X-PLOR Version 3.1 Manual, Yale University, New Haven, Conn.

51. Garrett, D. S., Kuszewski, J., Hancock, T. J., Lodi, P. J., Vuister, G. W., Gronenborn, A. M., and Clore, G. M. (1994) J. Magn. Reson., Ser. B 104, 99-103.

52. Kuszewski, J., Qin, J., Gronenborn, A. M., and Clore, G. M. (1995) J. Magn. Reson., Ser. B 106, 92-6.

53. Kuszewski, J., Gronenborn, A. M., and Clore, G. M. (1996) Protein Sci. 5, 1067-1080.

54. Kuszewski, J., Gronenborn, A. M., and Clore, G. M. (1997) J. Magn. Reson. 125, 171-177.

55. Clore, G. M., and Gronenborn, A. M. (1989) Crit. Rev. Biochem. Mol. Biol. 24, 479-564.

56. Wishart, D. S., and Sykes, B. D. (1994) Methods Enzymol. 239.

57. Chen, C. -K., Wieland, T., and Simon, M. I. (1996) Proc. Natl. Acad. Sci. U. S. A. 93, 12885-12889.

58. Nilges, M., Clore, G. M., and Gronenborn, A. M. (1988) Febs Lett 239, 129-36.

59. Brooks, B. R., Bruccoleri, R. E., Olafson, B. D., States, D. J., Swaminathan, S., and Karplus, M. (1983) J. Comput. Chem 4, 187-217.

60. Nilges, M., Clore, G. M., and Gronenborn, A. M. (1988) Febs Lett 229, 317-24.

61. Laskowski, R. A., MacArthur, M. W., Moss, D. S., and Thornton, J. M. (1993) J. Appl. Cryst. 26, 283-291.

62. Grzesiek, S. and Bax, A. (1992) J. Am. Chem. Soc.ds 114,6291-6293

63. Grzesiek, S. and Bax, A. (1992) J. Magn. Reson 99, 201-207

64. Grzesiek, S., Anglister, J. and Bax, A. (1993) J. Magn. Reson., Ser. B 101, 114-119

65. Grzesiek, S. and Bax, A. (1993) J. Biomol. Nmr 3, 185-204

66. Grzesiek, S. and Bax, A. (1992) J. Magn. Reson. 96, 432-40

67. Kay, L. E. et al. (1990) J. Magn. Reson. 89, 496-514

68. Ikura, M. et al. (1991) J. Biomol. Nmr, 299-304

69. Bax, A. et al. (1990) J. Magn. Reson. 88, 425-431

70. Powers, R. et al. (1992) Biochemistry 31,4334-4346

71. Friedrichs, M. S. et al. (1994) J. Biomol Nmr 4, 703-726

72. Wieland, T. Chen, C. K. (1999) Naunyn-Schmiedebergs Arch. Pharmacol 360,14-26.

73. Tesmer & Spray (1998) Curr Opinion Structural Biol. 8, 713-719

74. Wall et al. (1998) Structure 6, 1169-1183

75. Spray, S. R. (1997) Curr. Opn. St. Biol. 7, 849-856

76. Bax, A., Vuister, G. W., Grzesiek, S. and Delaglio (1994) Methods Enzymol. 239, 79-105

77. Clore, G. M. and Gronenborn, A. M. (1994) Methods Enzymol 239, 349-362

78. Wuthrich, K. (1986) NMR of proteins and nucleic acids. John Wiley & Sons, Inc., New York

79. DeVries et al. (1995) Proc. Natl. Acad. Sci. USA 92:11916-11920

80. Druey et al. (1996) Nature 379:742-746.

81. Hunt T. W. et al. (1996) Nature 383:175-177.

82. Koelle and Horvitz (1996) Cell 84:115-125

83. Hepler J. R. et al. (1997) Proc. Natl. Acad. Sci. USA. 94:428-432.

84. Shuey D J, Betty, M, Jones P G, Khawaja X V, Cockett, M. (1998) RGS7 attenuates signal transduction through the Gaq family of heterotrimeric G-proteins in mammalian cells. J. Neurochem. 70:1964-1972.

85. Berman D M, Kozasa T, Gilman A G. (1996) The GTPase activating protein RGS4 stabilizes the transition state for nucleotide hydrolysis J. Biol. Chem. 271:27909-27212.

86. Zheng, B. et al. (1999) TIBS 24 (November): 411-414.

87. de Alba, E. et al. (1999) J. Mol. Biol. 291:927-939.

88. Druey, K M. and Kehrl, J M (1999) J. Mol. Biology 291:927-939.

89. Wang et al. (1998) J. Biol. Chem. 273:26014-26025.

90. Hepler, J. R. (1999) TIBS 20(September) 376-382.

91. Berman, D M and Gilman, A G (1998) J. Biol. Chem. 273(3):1269-1272.

1. A representation of the three-dimensional solution structure of an RGS protein or portion thereof generated using the structural coordinates for RGS4-core protein.

2. The representation of claim 1 that is a representation of an RGS subfamily B protein.

3. The representation of claim 1 that is a representation of an RGS4.

4. The representation of claim 1 that is a representation of rat RGS4 .

5. The representation of claim 1 that is a representation of the Gα binding site of an RGS protein.

6. The representation of claim 1 that is a representation of the α₆-α₇ region of an RGS protein.

7. The representation of claim 1 that is a representation of the allosteric binding site in the α1-α2 region of an RGS protein.

8. The representation of claim 1 which comprises the entire core region of an RGS protein.

9. The representation of claim 1 that is generated using the structural coordinates for RGS4-core protein as determined by NMR spectroscopy.

10. A method for identifying, selecting or designing a chemical or biochemical species which is a modulator of RGS activity, RGS binding or RGS-Gα complex activity which comprised the steps:
(a) studying the interaction of one or more chemical or biochemical test species with the three-dimensional solution structure of an RGS4 protein or a portion thereof; and
(b) selecting a chemical or biochemical test species, which is predicted by its interaction with the three-dimensional structure of RGS4 to act as a modulator of an RGS protein to thereby identify, select or design the modulator.

11. The method of claim 10 wherein the modulator is identified, selected or designed based on its predicted interaction with a Gα binding site of a free RGS4 protein.

12. The method of claim 10 wherein the modulator is identified, selected or designed based on its predicted interaction with an allosteric binding site of a free RGS protein.

13. The method of claim 12 wherein the allosteric binding site is located in the α₁-α₂ region of a free RGS4 protein.

14. The method of claim 10 wherein the modulator is identified, selected or designed based on its predicted interaction with the α₆-α₇ region of a free RGS4 protein.

15. The method of claim 10 wherein the test species are selected from small organic molecules.

16. The method of claim 10 further comprising the steps of:
(a) obtaining the selected test species and
(b) assaying the test species to measure its activity as a modulator of RGS activity, RGS binding or RGS-Gα complex activity.

17. A modulator identified, selected or designed by the method of claim 10.

18. A process for identifying a substance that inhibits RGS activity, RGS binding or RGS-Gα complex activity comprising the step of determining the interaction between a candidate species and the structure of free RGS using a representation of the three-dimensional solution structure of RGS4.

19. A process for identifying a substance that mimics or promotes RGS activity, RGS binding or RGS-Gα complex activity comprising the step of determining the interaction between a candidate species and a representation of the three-dimensional structure of free RGS4.

20. A method of identifying modulators of RGS activity, RGS binding or RGS4/Gα complex activity by rational drug design comprising the steps:
(a) designing a potential modulator that will form a reversible or non-reversible bond with one or more amino acids in the RGS4 Gα binding site based upon the NMR structure coordinates of free RGS;
synthesizing or otherwise obtaining the modulator; and
(c) determining whether the potential modulator inhibits or promotes the activity of RGS or RGS4/G_{α} complex.

21. The method of claim 20 wherein said modulator is designed to interact with one or more atoms of said one or more amino acids in the RGS4 Gα binding site and wherein said one or more amino acids is selected from the group of D117, S118 or R121.

22. The method of claim 20 wherein the amino acids are selected from S39, E41, N42, L113, D117, S118, R121 or N82

23. A method for identifying modulators of RGS activity, RGS binding or RGS4/Gα complex activity by rational drug design comprising the steps:
(a) designing a potential modulator that will form a reversible or non-reversible bond with one or more amino acids in the allosteric binding site in the α1-α2 region of RGS based upon the NMR structure coordinates of free RGS4;
(b) synthesizing or otherwise obtaining the modulator; and
(c) determining whether the potential modulator inhibits or promotes the activity of RGS or RGS4/G_{α} complex.

24. The method of claim 23 wherein said modulator is designed to interact with one or more atoms of said one or more amino acids in the allosteric binding site and wherein said one or more atoms is selected from the group of RGS residues V10, W13, L17, 120, H23, E24, C25 and T132.

25. A modulator identified by the method of claim 23.

26. A method of identifying modulators of RGS activity, RGS binding or RGS4/Gα complex activity by rational drug design comprising the steps:
(a) designing a potential modulator that will form a reversible or non-reversible bond with one or more amino acids in the α₆-α₇ region of RGS4;
(b) synthesizing or otherwise obtaining the modulator; and
(c) determining whether the potential modulator inhibits or promotes the activity of RGS or RGS4-G_{α} complex.

27. The method of claim 26 wherein the modulator activity is assesses using an enzyme assay.

28. A method for identifying a potential modulator of RGS activity, RGS binding or RGS-Gα complex activity by rational drug design comprising the steps:

30. The method of claim 28 wherein the three dimensional structure of step (a) is that of free RGS4 as defined by the relative structural coordinates of RGS4-core protein according to Table 2, ± a root mean square deviation of not more than 1.5 Å from the conserved backbone atoms of the amino acids of RGS4-core.

31. A modulator identified by the method of claim 28.

32. The method of claim 28 wherein the three dimensional structure of step (a) is that of an RGS protein other than RGS4 and wherein the three dimensional structure of the RGS protein other than RGS4 is obtained by molecular replacement analysis or homology modeling techniques employing the relative structural coordinates of RGS4-core protein according to Table 2, ± a root mean square deviation of not more than 1.5 Å from the conserved backbone atoms of the amino acids of RGS4-core.

33. The method of claim 32 wherein the RGS protein other than RGS4 is an RGS subfamily B protein.

34. The method of claim 28 wherein the step of employing the three dimensional structure to designing or select the potential inhibitor comprises the steps of:
(1) identifying chemical or biochemical species or fragments thereof capable of binding to an RGS4 protein; and
(2) assembling the identified chemical entities or fragments into a single molecule to provide the structure of a potential inhibitor.

35. The method of claim 34 wherein in step (a) the chemical or biochemical species or fragments thereof capable of binding to the Gα binding site of a free RGS-core is a protein.

36. The method of claim 34 wherein in step (a) the chemical or biochemical species or fragments thereof capable of binding to the allosteric binding site in the α₁-α₂ region of a free RGS core protein are identified.

37. The method of claim 34 wherein in step (a) the chemical or biochemical species or fragments thereof capable of binding to α₆-α₇ region of a free RGS core protein are identified.

38. The method of claim 34 further comprising the step of testing the potential inhibitor designed or selected in step (b) as an modulator of an RGS protein.

39. A modulator identified by the method of claim 34.

40. A method for identifying a mutant of RGS4 where the biological activity of the derivative is different from that of RGS4 comprising the steps of:
(a) identifying amino acid residues of RGS4 protein that are involved in the function of the protein for regulation of G-protein signaling from the three dimensional structure of free RGS4;
(b) modifying one or more of the RGS4 amino acid residues identified in step (a) to generate the derivative of RGS4.

41. The method of claim 40 wherein the amino acid residues of RGS4 are modified by site directed mutagenesis of an RGS4 coding sequences after which the derivative RGS4 protein is expressed from the mutagenized RGS4 coding sequence.

42. The method of claim 40 wherein the amino acids modified are in the Gα binding site of RGS4.

43. The method of claim 40 wherein the amino acids modified are in an allosteric binding site of RGS4.

44. The method of claim 40 wherein the amino acids modified are in the α₆-α₇ region of RGS4.

45. A method for identifying potential modulators of an RGS protein which comprises the steps of:
(a) identifying an RGS binding site by detecting perturbations of the NMR resonances in NMR spectra of RGS4 core protein in the presence and absence of chemical and biochemcial species that potential bind to RGS4;
(b) employing the three dimensional structure of free RGS4 at the binding site identified in step (b) to select or design chemical or biochemical species that are predicted to bind at the binding site;
(c) testing the chemical or biochemical species that are predicted to bind at the binding site for function as an modulator of RGS activity or RGS-Gα complex activity.
